(19) United States
(12) Patent Application Publication
(10) Pub. No.: US 2024/0418723 A1
(43) Pub. Date: Dec. 19, 2024

(54) SEQUENTIAL TREATMENTS AND BIOMARKERS TO REVERSE RESISTANCE TO KINASE INHIBITORS

(71) Applicant: Queen Mary University of London, London (GB)

(72) Inventors: Pedro Rodriguez Cutillas, London (GB); Pedro Maria Casado-Izquierdo, London (GB); Federico Pedicona, London (GB); Maruan Hijazi, London (GB)

(21) Appl. No.: 18/702,488

(22) PCT Filed: Oct. 20, 2022
(86) PCT No.: PCT/EP22/79200

§ 371 Date: Apr. 18, 2024

Related U.S. Application Data
(60) Provisional application No. 63/270,727, filed on Oct. 22, 2021.

(30) Foreign Application Priority Data
Oct. 20, 2021 (GB) 2115017.2

(51) Int. Cl. G01N 33/574; A61K 31/4375; A61K 31/519; A61P 35/02; G01N 33/573; G01N 33/68

(52) U.S. Cl. CPC G01N 33/57426; A61K 31/4375; A61K 31/519; A61P 35/02; G01N 33/573; G01N 33/6893

(57) ABSTRACT

The invention relates generally to cancer treatments involving an epigenetic agent which promotes the effectiveness of a kinase inhibitor.

The invention relates generally to cancer treatments involving an epigenetic agent which promotes the effectiveness of a kinase inhibitor

The promise of anti-cancer drugs targeting oncogenic kinase signalling has not met original expectations; whilst some kinase inhibitors can significantly extend patient survival in some settings (e.g., chronic myeloid leukaemia treated with BCR-ABL targeted drugs), most of these agents produce transient responses and are only effective in small patient populations. This relatively low overall clinical efficacy has been ascribed to tumour heterogeneity and to the existence of pathways that compensate for target inhibition. For example, Balmanno et al Int J Cancer (2009) 125, 2332-2341 found that PI3K signalling has the potential to compensate for BRAF inhibition in colorectal cancer cells, whereas inhibition of PI3K/AKT activity has been shown to increase MEK/MAPK signalling by Hijazi et al. Nat Biotechnol. (2020) 38(4):493-502. It has been previously found by Casado, P. et al. Science signaling (2013) 6, rs6 and Dermit, M. et al. Oncogene (2017) 36, 2762-2774, that PKC isoforms and ERK1/2 were highly active in breast cancer cells and in primary AML cells resistant to PI3K inhibition, and a similar compensatory mechanism was noted by Elkabets et al (2015) Cancer Cell 27, 533-546 in drug-resistant oesophageal carcinomas, where an AXL-PKC axis acts in parallel to PI3K to activate mTOR. Similarly, FLT3/STAT, acting in parallel to MEK/ERK, was found to be highly active in primary AML cells resistant to MEK inhibition by Casado, P. et al. (2018) Leukemia 32, 1818-1822. However, although the biochemical pathways and circuitries that cause resistance to targeted drugs are starting to emerge, the underlying genetic and epigenetic causes that mediate the activation and formation of such compensatory pathways and network topologies are not well understood.

Epigenetic processes have recently emerged as potential mechanisms impacting intracellular cell signalling. Dysregulated histone methylation modifiers, in particular, have been directly and indirectly associated with oncogenic signalling pathways, thereby representing potential targets for therapeutic intervention. Smitheman et al. (2019), Haematologica 104(6) 1156-1167 describes how inactivation of the histone modifying enzyme lysine specific demethylase 1 (LSD1) by GSK2879552 enhances differentiation and promotes cytotoxic response when combined with all-trans retinoic acid (ATRA) in acute myeloid leukemia.

Here, we uncover a role of epigenetic chromatin modifiers in modulating kinase networks that mediate resistance to kinase inhibitor treatment. Our results provide a rationale for using epigenetic antagonists in order to overcome therapeutic resistance to kinase targeted drugs.

### SUMMARY OF THE INVENTION

According to a first aspect, the invention provides a method of treating cancer in a subject in need thereof, comprising administering to the subject an epigenetic agent which inhibits a chromatin-modifying enzyme and/or which promotes cellular differentiation, and then subsequently administering a kinase inhibitor to the subject.

According to a second aspect, the invention provides a method of sensitising a cancer to treatment with a kinase inhibitor wherein the cancer is pre-treated with an epigenetic agent which inhibits a chromatin-modifying enzyme and/or which promotes cellular differentiation.

According to a third aspect, the invention provides a kinase inhibitor for use in a method of treating cancer in a subject in need thereof, wherein the subject has previously been administered an epigenetic agent which inhibits a chromatin-modifying enzyme and/or which promotes cellular differentiation.

According to a fourth aspect, the invention provides an epigenetic agent for use in a method of sensitising a cancer to treatment with a kinase inhibitor wherein the method comprises pre-treating the cancer with an epigenetic agent which inhibits a chromatin-modifying enzyme and/or which promotes cellular differentiation.

According to a fifth aspect, the invention provides a kit comprising an epigenetic agent which inhibits a chromatin-modifying enzyme and/or which promotes cellular differentiation, and a kinase inhibitor for simultaneous, separate or sequential use for the treatment of cancer.

According to a sixth aspect, the invention provides a method of selecting a subject with cancer for treatment with an epigenetic agent which inhibits a chromatin-modifying enzyme and/or which promotes cellular differentiation, followed by treatment with a kinase inhibitor, wherein the method comprises:
detecting, in a sample of cancer cells obtained from the subject, the presence and/or level of one or more of the following markers:
   (i) phosphorylation at one or more of the following phosphorylation sites:
      Mitogen-activated protein kinase kinase kinase 2 phosphorylated at threonine 339;
      Ribosomal protein S6 kinase alpha-3 phosphorylated at threonine 391;
      Receptor tyrosine-protein kinase erbB-2 phosphorylated at serine 1073;
      Mitogen-activated protein kinase kinase kinase 3 phosphorylated at serine 129;
      Epidermal growth factor receptor phosphorylated at serine 991;
      PH domain leucine-rich repeat-containing protein phosphatase 1 phosphorylated at serine 1524;
      Dual specificity mitogen-activated protein kinase kinase 1 phosphorylated at serine 222;
      Dual specificity mitogen-activated protein kinase kinase 1 phosphorylated at serine 226;
      Kinase suppressor of Ras 1 phosphorylated at serine 202;
      Dual specificity mitogen-activated protein kinase kinase 1 phosphorylated at serine 230;
      Serine/threonine-protein kinase B-raf phosphorylated at threonine 753;
      Mitogen-activated protein kinase kinase kinase 4 phosphorylated at serine 499;
      Serine/threonine-protein kinase mTOR phosphorylated at threonine 2446;
      Serine/threonine-protein kinase mTOR phosphorylated at serine 2448;
      Serine/threonine-protein kinase A-Raf phosphorylated at serine 580;
      RAF proto-oncogene serine/threonine-protein kinase phosphorylated at serine 619;
      PH domain leucine-rich repeat-containing protein phosphatase 2 phosphorylated at serine 1210;
      Mitogen-activated protein kinase kinase kinase 20 at serine 648;
      Mitogen-activated protein kinase kinase kinase 20 at serine 649;
      Dual specificity mitogen-activated protein kinase kinase 2 phosphorylated at threonine 13;
      Glycogen synthase kinase-3 beta phosphorylated at serine 215;
      Glycogen synthase kinase-3 alpha phosphorylated at serine 278;
      Mitogen-activated protein kinase 3 phosphorylated at threonine 207;
      Serine/threonine-protein kinase N1 phosphorylated at serine 584;
      Tyrosine-protein phosphatase non-receptor type 7 phosphorylated at serine 359;
      RAC-alpha serine/threonine-protein kinase phosphorylated at serine 457;
      RAC-beta serine/threonine-protein kinase phosphorylated at serine 458;
      RAC-beta serine/threonine-protein kinase phosphorylated at tyrosine 438;
      Mitogen-activated protein kinase 3 phosphorylated at threonine 202;
      Mitogen-activated protein kinase 3 phosphorylated at tyrosine 204;
      Serine/threonine-protein kinase PAK 1 phosphorylated at serine 219;
      Serine/threonine-protein kinase PAK 1 phosphorylated at threonine 229;
      Serine/threonine-protein kinase PAK 1 phosphorylated at threonine 230;
      Eukaryotic translation initiation factor 4E-binding protein 1 phosphorylated at threonine 77; and/or
      Dual specificity mitogen-activated protein kinase kinase 1 phosphorylated at serine 377; and/or
   (ii) expression of one or more of the following proteins:
      Lysine-specific histone demethylase;
      Serine/threonine-protein kinase mTOR;
      Dual specificity mitogen-activated protein kinase kinase 2;
      Ribosomal protein S6 kinase beta-1;
      Mitogen-activated protein kinase 1;
      Dual specificity mitogen-activated protein kinase kinase 4;
      Ribosomal protein S6 kinase alpha-1;
      Dual specificity mitogen-activated protein kinase kinase 3;
      Ribosomal protein S6 kinase alpha-5;
      Dual specificity mitogen-activated protein kinase kinase 5;
      Phosphatidylinositol 4,5-bisphosphate 3-kinase catalytic subunit delta isoform; and/or
      Dual specificity mitogen-activated protein kinase kinase 1; and/or
   (iii) mutation of, or chromosomal rearrangement involving, one or more of the following genes: KMT2A, NPM1, KRAS, NRAS, DNMT3A, IDH2, TP53, TET2, FLT3 and/or STAG2; and/or
   (iv) classified as M5 by the French-American-British (FAB) AML classification scheme;
wherein detecting the presence and/or level of one or more of said markers selects the subject for treatment with the epigenetic agent followed by treatment with the kinase inhibitor.

### FIGURE LEGENDS

Reference is made to a number of Figures as follows:

FIG. 1.

Association between kinase network circuitry and cell differentiation marker expression in AML.

(A) Empirical cumulative distribution of phosphorylation sites markers of the AKT1/2-MTOR signaling axis in two AML patient samples. Each data-point is the quantification of one phosphosite as a function of their ranked intensities. Overall axis enrichment is calculated as the difference between the median of phosphorylation markers for the named signaling axes and the median of all phosphosites quantified within a tumor. (B) Expression of CD11b (left) and CD34 (right) as a function of AKT1/2-MTOR signaling axis (n=30 primary AML cases). (C) Comparison of edge (signaling axis) associated to CD11b or CD34. (D) Association between kinase axis enrichment and CD marker expression across 30 primary AML cases. R and p-values were calculated by Spearman rank (n=30 primary AML cases) and q-values obtained by the adjustment of p-values using the Benjamini-Hochberg procedure. (E) Number of signaling axes associated to the named CD markers at unadjusted p<0.01 and effect (slope)±0.5.

FIG. 2.

All trans retinoic acid and epigenetic inhibitors prime cell lines and primary AML for kinase inhibitor treatment.

(A) Screen scheme. (B) Cell viability as a function of trametinib (MEKi) treatment in P31/Fuj AML cells pre-treated with vehicle (ethanol) or ATRA (data-points are mean±SD, n=3 independent experiments). (C) Coefficient of drug interaction (CDI) values in −Log₂ scale (mean of n=3 technical replicates) for the named sequential treatments. (D) Log₂ CDI values from LSD1i treated cells followed by the named kinase inhibitors. Colour scale denotes CDI by viability and data point sizes are proportional to CDI by cell proliferation. (E-G) P31/Fuj AML cell number and apoptosis as a function of the indicated treatments. Dose-response curves are shown for the 5+3-day sequential treatment (E), the 3-day co-treatment (F) and the 3+5-day reverse treatment (G). Shown are the mean±SD of n=3 independent experiments; values from individual experiments are also shown. (H) Mean Log 2 CDI values (based on cell proliferation and viability) obtained from 17 primary AML cases treated sequentially with LSD1i and MEKi; data points in boxplots show median and interquartile ranges, n=3 technical replicates (independent treatments) per patient sample. (1) Mean Log 2 CDI values (based on cell proliferation and viability) obtained from 14 primary AML cases treated sequentially with LSD1i and PKC/FLT3i; data points in boxplots show median and interquartile ranges, n=3 technical replicates (independent treatments) per patient sample. PKC/FLT3i, midostaurin; PI3Ki, pictilisib; MEKi, trametinib; mTORi, Torin-1; LSD1i, GSK2879552; DNMTAi, decitabine; HDACi, vorinostat; EZH2i, CDI-1205; DOTL1i, pinometostat. Unless otherwise stated MEKi and LSD1i were used at 0.5 μM and the other inhibitors at 1 μM.

FIG. 3.

Multiomic analysis identifies mutations, proteins and phosphorylation sites associated to LSD1i->MEKi sequential treatment sensitivity in AML cell lines.

(A) Overview of genetic and cytogenetic markers and examples of proteins and phosphorylation sites associated to LSD1i->MEKi CDI values across 11 AML cell lines. Protein and phosphosite abundances are shown ranked 0 to 1 (proportional to colour and dot size). (B) Scatter plots showing relationship between average CDI values (based on proliferation, viability and apoptosis) and absolute basal abundances of selected proteins and phosphosites (as measured by mass spectrometry). Pearson's R and p-values are shown for each plot.

FIG. 4.

Multiomic analysis identifies mutations, proteins and phosphorylation sites associated to LSD1i->MEKi sensitivity in primary AML cases.

(A) Overview of genetic and cytogenetic markers and examples of proteins and phosphorylation sites associated to LSD1i->MEKi CDI values across 17 primary AML cases. Protein and phosphosite abundances are shown ranked 0 to 1 (proportional to colour and dot size). (B) Average CDI values as a function of NRAS, KRAS, DNMT3A or NPM1 mutations. p-values were calculated by unpaired two-sided t-test (n=12 NRAS WT; 5 NRAS MUT; 13 KRAS WT; 4 KRAS MUT; 14 DNMT3A WT; 3 DNMT3A MUT; 12 NPM1 WT; 5 NPM1 MUT). (C) Correlation plots showing relationship between average CDI values (based on proliferation and viability) and absolute basal abundances of selected proteins and phosphosites (as measured by mass spectrometry). Pearson's R and p-values are shown for each plot.

FIG. 5.

Integration of multiomic datasets of AML response to sequential treatment in a random forest (RF) machine learning model.

Integration of omics datasets was performed for the AML cell lines (A, C) and primary AML cases (B, D) tested. Shown are the RF-predicted CDI values vs the actual CDI values (A, B) and the ranked importance of individual predictors to the model (C, D).

FIG. 6.

LSD1i rewires the kinase network, reduces overall kinase pathway activation and induces a PI3K/AKT to MEK signaling switch.

(A) Experimental scheme of phosphoproteomic analysis of LSD1i->KIs treated cells. LSD1i was used at 0.5 μM and KIs at 1 μM. (B) Overview of phosphorylation sites modulated by the different kinase inhibitors in cells pre-treated with LSD1i or DMSO control (q-values were calculated from Limma p-values using the Benjamini-Hochberg FDR method, n=3 independent experiments in technical duplicate each). (C and D) Differences in network axes and kinase node centrality (using the page rank algorithm) in cells treated with LSD1i relative to control. Solid line is the regression line of the linear model. Dashed line denotes slope of 1. (E) Network axes modulated by the named kinase inhibitors in cells pre-treated or not with LSD1i. Slopes were calculated by linear regression. Dashed line denotes slope of 1. (F and G) Absolute abundance of the named phosphorylation sites. (H) Impact of kinase inhibitors on the named phosphorylation sites. Differences and p-values were calculated by Limma for each phosphosite as a function of kinase inhibitor treatment relative to the respective control. In F, G p-values were calculated by two-sided unpaired t-test (n=3 or 4 independent experiments).

FIG. 7.

Organelle proteomics identifies changes in the expression of MAPK and PI3K/AKT signaling proteins induced by LSD1.

(A) Scheme of subcellular proteomics analysis of LSD1i treated cells. A total of 5290 unique proteins were identified, and the top 3654 proteins for which at least two peptides were identified were selected for further analysis. (B) Examples of MEK/PI3K network protein changes induced by 5 days of LSD1i treatment in cytosol and nuclear/organelle fractions are shown. Boxplots show median and interquartile ranges of absolute protein abundances. p-values were calculated by two-sided unpaired t-test (n=4 independent experiments).

FIG. 8.

Associations between differentiation marker expression, kinase network circuitry, epigenetic reader/writer phosphorylation and kinase inhibitor sensitivity in primary AML cells.

(A) Association between CD marker expression and kinase centrality values across AML patients. (B) Association between CD marker expression and phosphorylated chromatin readers and writers. (C) Association between phosphorylated chromatin readers and writers and kinase centrality values. (D) Association between phosphorylated chromatin readers and writers and kinase axis enrichment. (E) Association between sensitivity to selected kinase inhibitors and CD marker expression. R and p-values were calculated by Spearman rank (n=30 primary AML cases) and q-values obtained by the adjustment of p-values using the Benjamini-Hochberg procedure.

FIG. 9.

Effect of all trans retinoic acid (ATRA) treatment on the signalling network of AML cells and sensitisation of primary AML cells to kinase inhibition by treatment with LSD1i.

(A) Impact of treatment with ATRA on PDTs (putative downstream targets) and kinase axis enrichment; differences and p-values were calculated by Limma as a function of ATRA treatment relative to control. (B) Examples of phosphorylation sites affected by ATRA treatment at different concentrations. Boxplots show median and interquartile ranges; p-values were calculated by two-sided unpaired t-test (n=4 independent experiments). Asterisks denote p-values by two-sided one sample t-test (n=3 for each patient sample, *p<0.05, **p<0.01, ***p<0.001).

FIG. 10.

LSD1i primes P31/Fuj AML cells for treatment with MEKi under a sequential treatment strategy.

(A-C) CDI values based on the cell number and apoptosis outputs for P31/Fuj cells treated with LSD1i and MEKi under a 5+3 days sequential treatment (A), 3-day co-treatment (B) or 3+5 days reverse treatment (C) strategy. (D-E) Dose-response curves and respective CDI values for P31/Fuj cells pre-treated for 5 days with the LSD1 inhibitors ORY-1001 (D) or CC-90011 (E), followed by 3-day treatment with MEKi (trametinib). Values in the dose-response curves are presented as the mean±SD (n=3 independent replicates). CDI values are expressed as Log₂ and are calculated based on the mean values of the respective dose-response curves.

FIG. 11.

FACS analysis of CD34-positive haematopoietic cells following sequential treatment with LSD1i and MEKi.

CD34-positive human haematopoietic cells were co-cultured with MS5 mouse bone marrow stromal cells and treated with either LSD1i or DMSO control for 5 days, followed by treatment with either MEKi or DMSO for an additional 3 days. Expression of selected differentiation and apoptosis markers was measured by flow cytometry. (A) Representative dot plots of cells gated according to CD34 vs CD45 expression. (B) Representative dot plots of cells sorted according to CD11b vs CD86 expression. (C) Representative dot plots of cells gated according to CD11b vs CD14 expression. (D) Representative dot plots of cells gated according to Annexin V binding. For each group, the proportion of the population positive for the marker indicated is summarised into bar charts, representing the mean proportion±S.D. (n=3 independent replicates). Asterisks denote p-values by two-sided unpaired t-test (n=3, **p<0.01, ***p<0.001).

FIG. 12.

FACS analysis of CD34-positive haematopoietic cells following sequential treatment with LSD1i and MEKi.

CD34-positive human haematopoietic cells were co-cultured with MS5 mouse bone marrow stromal cells and treated with either LSD1i or DMSO control for 5 days, followed by treatment with either MEKi or DMSO for an additional 3 days. Expression of selected differentiation and apoptosis markers was measured by flow cytometry. (A) Representative dot plots of cells sorted according to CD34 vs CD45 expression. (B) Representative dot plots of cells sorted according to CD11b vs CD14 expression. In each plot, cells that are also positive for Annexin V binding are highlighted in blue, whereas Annexin V negative cells are highlighted in red. Plots shown are representative of n=3 independent replicates.

FIG. 13.

AML cell line response to sequential treatment by mutational background.

Box plots summarizing the CDI values (based on proliferation, viability, apoptosis or an average of the previous three) for the LSD1i->MEKi sequential treatment in AML cells carrying a specific mutation (MUT, indicated above each panel) vs the wild-type (WT) ones. Statistical significance was calculated using two-sided unpaired t-test and resulting p-values (p) are displayed above each plot.

FIG. 14.

Correlation between AML cell line response to sequential treatment and basal expression of signalling proteins and phosphosites.

Correlation plots showing the relationship between average CDI values for each cell line (in response to LSD1i→MEKi) and the expression levels (as measured by mass spectrometry) of a selection of proteins and phosphosites. Pearson's R and p-values are indicated above each plot.

FIG. 15.

Primary AML response to sequential treatment by karyotype and FAB classification.

Box plots summarizing the average CDI values (based on proliferation and viability) for the LSD1i→MEKi sequential treatment in primary AML cases, grouped by karyotype (A) and FAB subgroup (B). Data points presented as the average of n=3 independent replicates. Statistical significance of differences between each group were calculated using two-sided unpaired t-test (p-values were not significant if not indicated on the plots).

FIG. 16.

Primary AML response to sequential treatment by mutational background.

Box plots summarizing the CDI values (based on proliferation, viability, apoptosis or an average of the previous three) for the LSD1i→MEKi sequential treatment in primary AML cases carrying a specific mutation (MUT, indicated above each panel) vs the wild-type (WT) ones. Statistical significance was calculated using two-sided unpaired t-test and resulting p-values (p) are displayed above each plot.

FIG. 17.

Correlation between primary AML response to sequential treatment and basal expression of signalling proteins and phosphosites.

Correlation plots showing the relationship between average CDI values for each primary AML case (in response to LSD1i→MEKi) and the expression levels (as measured by mass spectrometry) of a selection of proteins and phosphosites. Pearson's R and p-values are indicated above each plot.

FIG. 18.

LSD1i rewires the kinase network of AML cells.

(A) Qualitative data for phosphoproteomic analysis of AML cells treated with LSD1i and kinase inhibitors (KIs) sequentially. (B) Enrichment of selected kinase axes across AML cells pre-treated with either LSD1i or DMSO control followed by treatment with AZD5363 (AKTi), GDC0941 (PI3Ki), GDC0994 (ERKi), trametinib (MEKi) or midostaurin (PKC/FLT3i). Data points in boxplots show median and interquartile ranges; p-values were calculated by two-sided unpaired t-test (n=3 independent experiments). (C) Comparison of phosphorylation sites modulated (Limma p-values <0.1) by the named kinase inhibitors in cells pre-treated with DMSO control vs LSD1i. Slopes were calculated by linear regression; dashed line denotes slope=1.

FIG. 19.

LSD1i rewires the kinase network of AML cells.

(A) Absolute abundances of the named phosphorylation sites across AML cells pre-treated with either LSD1i or DMSO control followed by treatment with the kinase inhibitors indicated. Data points in boxplots show median and interquartile ranges; p-values were calculated by two-sided unpaired t-test (n=3 independent experiments). (B to E) Impact of kinase inhibitors on phosphorylation sites associated with activity of the PI3K/AKT/MTOR (B) and RAF/MEK/ERK (C) signalling pathways, and with CDKs and cyclins (D) and proteins involved in DNA repair (E) in cells pre-treated with LSD1i or DMSO control. Differences and p-values were calculated by Limma for each phosphosite as a function of kinase inhibitor treatment relative to the respective control (n=3 independent experiments).

FIG. 20.

Fraction controls for nuclear/organelle & cytosolic proteomics of AML cells following LSD1i.

(A and B) List of Location Ontologies enriched in the nuclear/organelle (A) or cytosolic (B) fractions. Enrichment values and p-values were calculated using a modified Fisher's Exact test (n=4); cut-offs of p-value <0.01 and enrichment >2 were applied. (C) Selection of protein markers for a variety of subcellular locations, showing an enrichment of nuclear and organelle markers in the nuclear fraction and an enrichment of glycolytic enzymes (cytosolic protein markers) in the cytosolic fraction. Box plots show median and interquartile ranges of Log₂ absolute protein abundances (n=4 independent replicates).

FIG. 21.

Effect of LSD1i on the nuclear/organelle and cytosolic proteome of AML cells.

(A and B) Volcano plots showing changes in abundance of individual proteins in the cytosolic (A) and nuclear/organelle (B) fractions of cells treated with LSD1i relative to control. Adjusted p-value (q-value)<0.05 and Log₂ fold changes >0.5 or <−0.5 were applied as cut-offs. (C and D) NCI pathway enrichment of proteins decreased or increased in the cytosolic (C) or nuclear/organelle (D) fractions in LSD1i-treated vs control. Enrichment values and p-values were calculated using hypergeometric test (n=4 independent replicates).

FIG. 22.

Effect of LSD1i on the nuclear/organelle and cytosolic proteome of AML cells.

Examples of changes in protein levels induced by LSD1i treatment in cytosolic and nuclear/organelle fractions. Selected proteins are categorised under kinases and signalling regulators (A) and integrins and cell-to-cell interactions (B). Boxplots show median and interquartile ranges; p-values were calculated by two-sided unpaired t-test (n=4 independent replicates).

FIG. 23.

Effect of LSD1i on the nuclear/organelle and cytosolic proteome of AML cells.

Examples of transcription factors and transcription regulators whose expression is affected by LSD1i treatment in cytosolic and nuclear/organelle fractions. Boxplots show median and interquartile ranges; p-values were calculated by two-sided unpaired t-test (n=4 independent replicates).

FIG. 24.

Effect of LSD1i on the nuclear/organelle and cytosolic proteome of AML cells.

Examples of chromatin associated proteins whose expression is affected by LSD1i treatment in cytosolic and nuclear/organelle fractions. Boxplots show median and interquartile ranges; p-values were calculated by two-sided unpaired t-test (n=4 independent replicates).

### DETAILED DESCRIPTION OF THE INVENTION

According to a first aspect, the invention provides a method of treating cancer in a subject in need thereof, comprising administering to the subject an epigenetic agent which inhibits a chromatin-modifying enzyme and/or which promotes cellular differentiation, and then subsequently administering a kinase inhibitor to the subject.

As used herein, the term “patient” and the term “subject” are used interchangeably. The patient may or may not have received any previous treatment for a cancer, such as AML. The patient may be termed an “individual” patient.

According to a second aspect, the invention provides a method of sensitising a cancer to treatment with a kinase inhibitor wherein the cancer is pre-treated with an epigenetic agent which inhibits a chromatin-modifying enzyme and/or which promotes cellular differentiation.

As used herein, the term “sensitising a cancer” refers to making a cancer more susceptible to a treatment. When a cancer has been sensitised, the effect of one or more other treatment may be enhanced. Typically a first treatment sensitises the cancer to a second treatment. The effect of the second treatment may then be enhanced. The first treatment may be an epigenetic agent as defined herein. The second treatment may be a kinase inhibitor as defined herein. Without being bound by theory, the first treatment may affect cancer cells by causing them to acquire signalling topologies associated with sensitivity to the second treatment. This may reinforce the effects of the second treatment by preventing cancer cells from activating compensatory pathways, which may have reduced the effectiveness of the second treatment. For example, the first treatment may be an epigenetic inhibitor such as an LSD1i (for example GSK-2879552 or ATRA) which may sensitise a cancer (such as AML) to a second treatment which may be a kinase inhibitor such as an inhibitor of MEK1 or MEK 2 (for example trametinib).

As used herein, the term “pre-treating” or “pre-treated” refers to a first treatment being administered prior to a second treatment. The first treatment may be an epigenetic agent as defined herein. The second treatment may be a kinase inhibitor as defined herein. There may be a period between the administration of the first treatment and the administration of the second treatment. The period between administration of the first treatment and the administration of the second treatment may be of any suitable duration and may be determined by a clinician. For example, the epigenetic agent may be administered up to 1 month before administration of the kinase inhibitor. The epigenetic agent may be administered up to 3 weeks, up to 2 weeks or up to 1 week before administration of the kinase inhibitor. The epigenetic agent may be administered up to 7 days, up to 6 days, up to 5 days, up to 4 days, up to 3 days, up to 2 days or up to 1 day before administration of the kinase inhibitor. The epigenetic agent may be administered more than once. The pre-treating may therefore involve a pre-treatment phase comprising one or more administrations of the first treatment, such as the epigenetic agent. The pre-treatment phase may be followed by a treatment phase comprising one or more administrations of the second treatment, such as the kinase inhibitor. The epigenetic agent may be first administered up to 3 weeks, up to 2 weeks or up to 1 week before the first administration of the kinase inhibitor. The epigenetic agent may be first administered up to 7 days, up to 6 days, up to 5 days, up to 4 days, up to 3 days, up to 2 days or up to 1 day before the first administration of the kinase inhibitor. The epigenetic agent may be last administered up to 3 weeks, up to 2 weeks or up to 1 week before the first administration of the kinase inhibitor. The epigenetic agent may be last administered up to 7 days, up to 6 days, up to 5 days, up to 4 days, up to 3 days, up to 2 days or up to 1 day before the first administration of the kinase inhibitor.

As used herein, a “sequential treatment” refers to a treatment wherein a cancer has been sensitised as described herein or a treatment wherein a cancer has been pre-treated as described herein. Typically, a sequential treatment comprises administering an epigenetic agent as described herein prior to administering a kinase inhibitor as described herein.

According to a third aspect, the invention provides a kinase inhibitor for use in a method of treating cancer in a subject in need thereof, wherein the subject has previously been administered an epigenetic agent which inhibits a chromatin-modifying enzyme and/or which promotes cellular differentiation.

According to a fourth aspect, the invention provides an epigenetic agent for use in a method of sensitising a cancer to treatment with a kinase inhibitor wherein the method comprises pre-treating the cancer with an epigenetic agent which inhibits a chromatin-modifying enzyme and/or which promotes cellular differentiation.

According to a fifth aspect, the invention provides a kit comprising an epigenetic agent which inhibits a chromatin-modifying enzyme and/or which promotes cellular differentiation, and a kinase inhibitor for simultaneous, separate or sequential use for the treatment of cancer.

As used herein, a “kit” is a packaged combination optionally including instructions for use of the combination and/or other reactions and components for such use. The kit may be a product comprising the epigenetic agent and the kinase inhibitor and may be described as a “combined product” accordingly. The epigenetic agent and the kinase inhibitor may therefore be presented side by side and can therefore be applied simultaneously, separately or sequential (ie at intervals) to one and the same human or animal body. The kit may comprise one or more devices, such as injection devices for storing and/or administering the epigenetic agent and/or the kinase inhibitor. The kit may comprise a first separate device storing and/or administering the epigenetic agent and a second device for storing and/or administering the kinase inhibitor. The epigenetic agent may be contained in a vial such as an ampoule. The kinase inhibitor may be contained in a vial such as an ampoule.

As used herein, for simultaneous, separate or sequential use refers to the suitability of the kit to allowing the epigenetic agent and the kinase inhibitor to be administered simultaneously, separately or sequential (ie at intervals) to one and the same human or animal body. The use of the terms “separate or sequential” is used to indicate the epigenetic agent and the kinase inhibitor are not necessarily present as a union (eg as components of the same composition) but may instead be present in a side-by-side presentation as part of kit.

The methods of the invention may comprise determining the level of the one or more proteins and/or the level of phosphorylation at the one or more phosphorylation sites. The determining may be by any suitable means such as by an assay described herein. The assay may for instance be an LC-MS/MS assay or an assay based on affinity reagents such as aptamers, molecularly imprinted polymers, or antibodies (immunochemical assays). The assay based on affinity reagents may be a Western blot assay, an ELISA assay or a reversed phase protein assay.

The methods of the invention may comprise obtaining the sample from the patient.

The cancer may comprise cells with one or more of the following markers:
(i) phosphorylation at one or more of the following phosphorylation sites:
   Mitogen-activated protein kinase kinase kinase 2 phosphorylated at threonine 339;
   Ribosomal protein S6 kinase alpha-3 phosphorylated at threonine 391;
   Receptor tyrosine-protein kinase erbB-2 phosphorylated at serine 1073;
   Mitogen-activated protein kinase kinase kinase 3 phosphorylated at serine 129;
   Epidermal growth factor receptor phosphorylated at serine 991;
   PH domain leucine-rich repeat-containing protein phosphatase 1 phosphorylated at serine 1524;
   Dual specificity mitogen-activated protein kinase kinase 1 phosphorylated at serine 222;
   Dual specificity mitogen-activated protein kinase kinase 1 phosphorylated at serine 226;
   Kinase suppressor of Ras 1 phosphorylated at serine 202;
   Dual specificity mitogen-activated protein kinase kinase 1 phosphorylated at serine 230;
   Serine/threonine-protein kinase B-raf phosphorylated at threonine 753;
   Mitogen-activated protein kinase kinase kinase 4 phosphorylated at serine 499;
   Serine/threonine-protein kinase mTOR phosphorylated at threonine 2446;
   Serine/threonine-protein kinase mTOR phosphorylated at serine 2448;
   Serine/threonine-protein kinase A-Raf phosphorylated at serine 580;
   RAF proto-oncogene serine/threonine-protein kinase phosphorylated at serine 619;
   PH domain leucine-rich repeat-containing protein phosphatase 2 phosphorylated at serine 1210;
   Mitogen-activated protein kinase kinase kinase 20 at serine 648;
   Mitogen-activated protein kinase kinase kinase 20 at serine 649;
   Dual specificity mitogen-activated protein kinase kinase 2 phosphorylated at threonine 13;
   Glycogen synthase kinase-3 beta phosphorylated at serine 215;
   Glycogen synthase kinase-3 alpha phosphorylated at serine 278;
   Mitogen-activated protein kinase 3 phosphorylated at threonine 207;
   Serine/threonine-protein kinase N1 phosphorylated at serine 584;
   Tyrosine-protein phosphatase non-receptor type 7 phosphorylated at serine 359;
   RAC-alpha serine/threonine-protein kinase phosphorylated at serine 457;
   RAC-beta serine/threonine-protein kinase phosphorylated at serine 458;
   RAC-beta serine/threonine-protein kinase phosphorylated at tyrosine 438;
   Mitogen-activated protein kinase 3 phosphorylated at threonine 202;
   Mitogen-activated protein kinase 3 phosphorylated at tyrosine 204;
   Serine/threonine-protein kinase PAK 1 phosphorylated at serine 219;
   Serine/threonine-protein kinase PAK 1 phosphorylated at threonine 229;
   Serine/threonine-protein kinase PAK 1 phosphorylated at threonine 230;
   Eukaryotic translation initiation factor 4E-binding protein 1 phosphorylated at threonine 77; and/or
   Dual specificity mitogen-activated protein kinase kinase 1 phosphorylated at serine 377; and/or
(ii) expression of one or more of the following proteins:
   Lysine-specific histone demethylase;
   Serine/threonine-protein kinase mTOR;
   Dual specificity mitogen-activated protein kinase kinase 2;
   Ribosomal protein S6 kinase beta-1;
   Mitogen-activated protein kinase 1;
   Dual specificity mitogen-activated protein kinase kinase 4;
   Ribosomal protein S6 kinase alpha-1;
   Dual specificity mitogen-activated protein kinase kinase 3;
   Ribosomal protein S6 kinase alpha-5;
   Dual specificity mitogen-activated protein kinase kinase 5;
   Phosphatidylinositol 4,5-bisphosphate 3-kinase catalytic subunit delta isoform; and/or
   Dual specificity mitogen-activated protein kinase kinase 1; and/or
(iii) mutation of, or chromosomal rearrangement involving, one or more of the following genes: KMT2A, NPM1, KRAS, NRAS, DNMT3A, IDH2, TP53, TET2, FLT3 and/or STAG2; and/or
(iv) classified as M5 by the French-American-British (FAB) AML classification scheme.

The method of the invention may be described as a method for treating a patient with sequential-treatment, wherein the patient is suffering from cancer, the method comprising the steps of:
(a) determining whether the patient is a predicted sequential-treatment-responder by
   (i) obtaining or having obtained a biological sample from the patient; and
   (ii) performing or having performed a proteomic assay and/or a phosphoproteomic assay on the biological sample to determine if the patient has a sequential-treatment-responder phenotype; and
(b) if the patient has a sequential-treatment-responder phenotype, then administering sequential-treatment to the patient and if the patient does not have a sequential-treatment-responder phenotype then not administering sequential-treatment or administering a different treatment for cancer.

The proteomic assay and/or a phosphoproteomic assay may be any suitable assay including any such assay described herein. The proteomic assay may determine the level of one or more of the biomarkers described herein.

The biomarkers described herein include the proteins indicated in Table 1, each of which may be referred to by either the “full protein name” or by the corresponding entry in the “signatures” column. The “increased in” column indicates whether the biomarker is typically increased in cells sensitive to sequential treatment (“sensitive”) or in cells resistant to sequential treatment (“resistant”).

As used herein, the term “cells sensitive to sequential treatment” typically refers to cells from a patient who is responding or will respond to treatment with a sequential treatment disclosed herein. Likewise, the term “cells resistant to sequential treatment” typically refers to a patient who is not responding or will not respond to treatment with a sequential treatment disclosed herein. Responding here means there is sign of clinical improvement, a cessation of clinical deterioration or a slowed rate of clinical deterioration.

TABLE 1

| UniProt ID of | Version number | | | |
|---|---|---|---|---|
| canonical sequence | and date | Full protein name | Signatures | increased in: |
| P42345/MTOR_HUMAN | Entry version 222 | Serine/threonine-protein kinase mTOR | MTOR | Resistant |
| | (2 Jun. 2021) | | | |
| P36507/MP2K2_HUMAN | Entry version 231 | Dual specificity mitogen-activated | MP2K2 | Sensitive |
| | (2 Jun. 2021) | protein kinase kinase 2 | | |
| P23443/KS6B1_HUMAN | Entry version 216 | Ribosomal protein S6 kinase beta-1 | KS6B1 | Sensitive |
| | (2 Jun. 2021) | | | |
| P28482/MK01_HUMAN | Entry version 238 | Mitogen-activated protein kinase 1 | MK01 | Sensitive |
| | (2 Jun. 2021) | | | |
| P45985/MP2K4_HUMAN | Entry version 208 | Dual specificity mitogen-activated | MP2K4 | Sensitive |
| | (2 Jun. 2021) | protein kinase kinase 4 | | |
| Q15418/KS6A1_HUMAN | Entry version 219 | Ribosomal protein S6 kinase alpha-1 | KS6A1 | Sensitive |
| | (2 Jun. 2021) | | (p90 S6K) | |
| P46734/MP2K3_HUMAN | Entry version 212 | Dual specificity mitogen-activated | MAP2K3 | Sensitive |
| | (2 Jun. 2021) | protein kinase kinase 3 | | |
| O75582/KS6A5_HUMAN | Entry version 208 | Ribosomal protein S6 kinase alpha-5 | KS6A5 | Resistant |
| | (2 Jun. 2021) | | | |
| Q13163/MP2K5_HUMAN | Entry version 207 | Dual specificity mitogen-activated | MAP2K5 | Resistant |
| | (2 Jun. 2021) | protein kinase kinase 5 | | |
| O60341/KDM1A_HUMAN | Entry version 213 | Lysine-specific histone demethylase | KDM1A | Sensitive |
| | (2 Jun. 2021) | | | |
| O00329/PK3CD_HUMAN | Entry version 200 | Phosphatidylinositol 4,5-bisphosphate | PK3CD | Sensitive |
| | (2 Jun. 2021) | 3-kinase catalytic subunit delta isoform | | |
| Q02750/MP2K1_HUMAN | Entry version 226 | Dual specificity mitogen-activated | MAP2K1 | Sensitive |
| | (2 Jun. 2021) | protein kinase kinase 1 | | |

The biomarkers described herein include the phosphorylation sites indicated in Table 2, each of which may be referred to by either the “full phosphorylation site name” or by the corresponding entry in the “signatures” column. The “increased in” column indicates whether the biomarker is typically increased in cells sensitive to sequential treatment (“sensitive”) or in cells resistant to sequential treatment (“resistant”). As used herein, the residue numbering of the phosphorylation site(s) corresponds to the residue numbering in the UniProt ID of the canonical sequence with the version number and date indicated. All protein sequences start from the methionine 1 position for each protein listed. For each phosphorylation site/signature a number of “Peptide with alternative phosphorylation sites” are given; all possible combinations of phosphorylation sites embraced by the “Peptide with alternative phosphorylation sites” column are explicitly contemplated herein. Accordingly, any reference to a phosphorylation site or signature may be replaced by a reference to the corresponding entry in the “Peptide with alternative phosphorylation sites” column, or any one or more of the phosphorylation sites embraced “Peptide with alternative phosphorylation sites” column. Without being bound by theory, the phosphorylation site given in the “full phosphorylation site name” column is the preferred phosphorylation site of the phosphorylation sites embraced “Peptide with alternative phosphorylation sites” column. Further details on the peptides and phosphorylation sites referred to in the “Peptide with alternative phosphorylation sites” are given in Table 3, wherein the “Gene” column refers to the name of a gene also provided in the “Signatures” column of Table 2, wherein the UniProt ID of the canonical sequence, version number and date and name of the protein (as given in the “fully phosphorylation site name” column of Table 2, albeit there for only a single phosphorylation site) correspond to those in Table 2. Biomarkers according to the invention include any peptide of Table 3 phosphorylated at any one of the residues belonging to that peptide recited in Table 3. Biomarkers according to the invention include any peptide of Table 3 phosphorylated at any two of the residues belonging to that peptide recited in Table 3. Biomarkers according to the invention include any peptide of Table 3 phosphorylated at any three of the residues belonging to that peptide recited in Table 3. Biomarkers according to the invention include any peptide of Table 3 phosphorylated at any 4, 5, 6, 7, 8, 9, 10, 11, 12 or 13 of the residues belonging to that peptide recited in Table 3. Biomarkers of the invention include any expression, mutation or chromosomal rearrangement signatures recited in Table 3. Any reference herein to a biomarker found within Table 3 may be substituted for any alternative biomarker found within the same row of Table 3. Biomarkers according to the invention may include any one of the phosphorylation sites recited in Table 4, wherein the “Signatures” column refers to the name of a gene also provided in the “Signatures” column of Table 2, wherein the UniProt ID of the canonical sequence, version number and date and name of the protein (as given in the “fully phosphorylation site name” column of Table 2, albeit there for only a single phosphorylation site) correspond to those in Table 2. Biomarkers according to the invention include any peptide of Table 4 phosphorylated at any one of the residues belonging to that peptide recited in Table 4. Biomarkers according to the invention include any peptide of Table 4 phosphorylated at any two of the residues belonging to that peptide recited in Table 4. Biomarkers according to the invention include any peptide of Table 4 phosphorylated at any three of the residues belonging to that peptide recited in Table 4. Biomarkers according to the invention include any peptide of Table 4 phosphorylated at any 4, 5, 6, 7, 8, 9, 10, 11, 12 or 13 of the residues belonging to that peptide recited in Table 4. Biomarkers of the invention include any expression, mutation or chromosomal rearrangement signatures recited in Table 4.

TABLE 2

| | | | Peptide | | |
|---|---|---|---|---|---|
| UniProt ID | | Full | with | | |
| of | Version number | phosphorylation | alternative | | |
| canonical | and | site | phosphorylation | | increased |
| sequence | date | name | sites | Signatures | in: |
| Q9Y2U5/ | Entry version 210 | Mitogen-activated | RGSDIDN | MAP3K2 | Sensitive |
| M3K2_HUMAN | (2 Jun. 2021) | protein | PTLTVMD | at | |
| | | kinase | ISPPSR | T339 | |
| | | kinase | (SEQ ID | | |
| | | kinase | NO: 1) | | |
| | | 2 | | | |
| | | phosphorylated | | | |
| | | at | | | |
| | | threonine | | | |
| | | 339 | | | |
| P51812/ | Entry version 214 | Ribosomal | GFSFVAI | RPS6KA3 | Sensitive |
| KS6A3_HUMAN | (2 Jun. 2021) | protein | TSDDESQ | at | |
| | | S6 | AMQTVGV | T391 | |
| | | kinase | HSIVQQL | | |
| | | alpha-3 | HR | | |
| | | phosphorylated | (SEQ ID | | |
| | | at | NO: 2) | | |
| | | threonine | | | |
| | | 391 | | | |
| P04626/ | Entry version 260 | Receptor | SPLAPSE | ERBB2 | Sensitive |
| ERBB2_HUMAN | (2 Jun. 2021) | tyrosine-protein | GAGSDVF | at | |
| | | kinase | DGDLGMG | S1073 | |
| | | erbB-2 | AAK | | |
| | | phosphorylated | (SEQ ID | | |
| | | at | NO: 3) | | |
| | | serine | | | |
| | | 1073 | | | |
| Q99759/ | Entry version 212 | Mitogen-activated | NHNSSSP | MAP3K3 | Sensitive |
| M3K3_HUMAN | (2 Jun. 2021) | protein | HSGVSR | at | |
| | | kinase | (SEQ ID | S129 | |
| | | kinase | NO: 4) | | |
| | | kinase | | | |
| | | 3 | | | |
| | | phosphorylated | | | |
| | | at | | | |
| | | serine | | | |
| | | 129 | | | |
| P00533/ | Entry version 278 | Epidermal | MHLPSPT | EGFR | Sensitive |
| EGFR_HUMAN | (2 Jun. 2021) | growth | DSNFYR | at | |
| | | factor | (SEQ ID | S991 | |
| | | receptor | NO: 5) | | |
| | | phosphorylated | | | |
| | | at | | | |
| | | serine | | | |
| | | 991 | | | |
| O60346/ | Entry version 190 | PH | CMLHPIC | PHLPP1 | Sensitive |
| PHLP1_HUMAN | (2 Jun. 2021) | domain | LSNSFOR | at | |
| | | leucine-rich | | S1524 | |
| | | repeat-containing | (SEQ ID | | |
| | | protein | NO: 6) | | |
| | | phosphatase | | | |
| | | 1 | | | |
| | | phosphorylated | | | |
| | | at | | | |
| | | serine | | | |
| | | 1524 | | | |
| Q02750/ | Entry version 226 | Dual | LCDFGVS | MAP2K1 | Sensitive |
| MP2K1_HUMAN | (2 Jun. 2021) | specificity | GQLIDSM | at | |
| | | mitogen-activated | ANSFVGT | S222 | |
| | | protein | R | | |
| | | kinase | (SEQ ID | | |
| | | kinase | NO: 7) | | |
| | | 1 | | | |
| | | phosphorylated | | | |
| | | at | | | |
| | | serine | | | |
| | | 222 | | | |
| Q02750/ | Entry version 226 | Dual | LCDFGVS | MAP2K1 | Sensitive |
| MP2K1_HUMAN | (2 Jun. 2021) | specificity | GQLIDSM | at | |
| | | mitogen-activated | ANSFVGT | S226 | |
| | | protein | R | | |
| | | kinase | (SEQ ID | | |
| | | kinase | NO: 8) | | |
| | | 1 | | | |
| | | phosphorylated | | | |
| | | at | | | |
| | | serine | | | |
| | | 226 | | | |
| Q8IVT5/ | Entry version 174 | Kinase | RESGSGP | KSR1 | Sensitive |
| KSR1_HUMAN | (2 Jun. 2021) | suppressor | STDTLSA | at | |
| | | of | ASLPWPP | S202 | |
| | | Ras | GSSQLGR | | |
| | | 1 | | | |
| | | phosphorylated | (SEQ ID | | |
| | | at | NO: 9) | | |
| | | serine | | | |
| | | 202 | | | |
| Q02750/ | Entry version 226 | Dual | LCDFGVS | MAP2K1 | Sensitive |
| MP2K1_HUMAN | (2 Jun. 2021) | specificity | GQLIDSM | at | |
| | | mitogen-activated | ANSFVGT | S230 | |
| | | protein | R | | |
| | | kinase | (SEQ ID | | |
| | | kinase | NO: 10) | | |
| | | 1 | | | |
| | | phosphorylated | | | |
| | | at | | | |
| | | serine | | | |
| | | 230 | | | |
| P15056/ | Entry version 246 | Serine/threonine- | TPIQAGG | BRAF | Resistant |
| BRAF_HUMAN | (2 Jun. 2021) | protein | YGAFPVH | at | |
| | | kinase | | T753 | |
| | | B-raf | (SEQ ID | | |
| | | phosphorylated | NO: 11) | | |
| | | at | | | |
| | | threonine | | | |
| | | 753 | | | |
| Q9Y6R4/ | Entry version 199 | Mitogen-activated | LESEDDS | MAP3K4 | Resistant |
| M3K4_HUMAN | (2 Jun. 2021) | protein | LGWGAPD | at | |
| | | kinase | WSTEAGF | S499 | |
| | | kinase | SR | | |
| | | kinase | (SEQ ID | | |
| | | 4 | NO: 12) | | |
| | | phosphorylated | | | |
| | | at | | | |
| | | serine | | | |
| | | 499 | | | |
| P42345/ | Entry version 222 | Serine/threonine- | TDSYSAG | MTOR | Resistant |
| MTOR_HUMAN | (2 Jun. 2021) | protein | QSVEILD | at | |
| | | kinase | GVELGEP | T2446 | |
| | | mTOR | AHKK | | |
| | | phosphorylated | (SEQ ID | | |
| | | at | NO: 13) | | |
| | | threonine | | | |
| | | 2446 | | | |
| P42345/ | Entry version 222 | Serine/threonine- | TDSYSAG | MTOR | Resistant |
| MTOR_HUMAN | (2 Jun. 2021) | protein | QSVEILD | at | |
| | | kinase | GVELGEP | S2448 | |
| | | mTOR | AHKK | | |
| | | phosphorylated | (SEQ ID | | |
| | | at | NO: 14) | | |
| | | serine | | | |
| | | 2448 | | | |
| P10398/ | Entry version 230 | Serine/threonine- | SASEPSL | ARAF | Resistant |
| ARAF_HUMAN | (2 Jun. 2021) | protein | HR | at | |
| | | kinase | (SEQ ID | S580 | |
| | | A-Raf | NO: 15) | | |
| | | phosphorylated | | | |
| | | at | | | |
| | | serine | | | |
| | | 580 | | | |
| P04049/ | Entry version 230 | RAF | SASEPSL | RAF1 | Resistant |
| RAF1_HUMAN | (2 Jun. 2021) | proto-oncogene | HR | at | |
| | | serine/threonine- | (SEQ ID | S619 | |
| | | protein | NO: 16) | | |
| | | kinase | | | |
| | | phosphorylated | | | |
| | | at | | | |
| | | serine | | | |
| | | 619 | | | |
| Q6ZVD8/ | Entry version 163 | PH | QNSVNSG | PHLPP2 | Resistant |
| PHLP2_HUMAN | (2 Jun. 2021) | domain | MLLPMSK | at | |
| | | leucine-rich | | S1210 | |
| | | repeat-containing | (SEQ ID | | |
| | | protein | NO: 17) | | |
| | | phosphatase | | | |
| | | 2 | | | |
| | | phosphorylated | | | |
| | | at | | | |
| | | serine | | | |
| | | 1210 | | | |
| Q9NYL2/ | Entry version 185 | Mitogen-activated | NFSSLHL | MAP3K20 | Resistant |
| M3K20_HUMAN | (2 Jun. 2021) | protein | NSR | at | |
| | | kinase | (SEQ ID | S648 | |
| | | kinase | NO: 18) | | |
| | | kinase | | | |
| | | 20 | | | |
| | | at | | | |
| | | serine | | | |
| | | 648 | | | |
| Q9NYL2/ | Entry version 185 | Mitogen-activated | NFSSLHL | MAP3K20 | Resistant |
| M3K20_HUMAN | (2 Jun. 2021) | protein | NSR | at | |
| | | kinase | (SEQ ID | S649 | |
| | | kinase | NO: 19) | | |
| | | kinase | | | |
| | | 20 | | | |
| | | at | | | |
| | | serine | | | |
| | | 649 | | | |
| P36507/ | Entry version 231 | Dual | RKPVLPA | MAP2K2 | Sensitive |
| MP2K2_HUMAN | (2 Jun. 2021) | specificity | LTINPTI | (MEK2) | |
| | | mitogen-activated | AEGPSPT | at | |
| | | protein | SEGASEA | T13 | |
| | | kinase | NLVDLQK | | |
| | | kinase | | | |
| | | 2 | (SEQ ID | | |
| | | phosphorylated | NO: 20) | | |
| | | at | | | |
| | | threonine | | | |
| | | 13 | | | |
| P49841/ | Entry version 252 | Glycogen | QLVRGEP | GSK3B | Resistant |
| GSK3B_HUMAN | (2 Jun. 2021) | synthase | NVSYICS | at | |
| | | kinase-3 | R | S215 | |
| | | beta | (SEQ ID | | |
| | | phosphorylated | NO: 21) | | |
| | | at | | | |
| | | serine | | | |
| | | 215 | | | |
| P49840/ | Entry version 206 | Glycogen | QLVRGEP | GSK3A | Resistant |
| GSK3A_HUMAN | (2 Jun. 2021) | synthase | NVSYICS | at | |
| | | kinase-3 | R | S278 | |
| | | alpha | (SEQ ID | | |
| | | phosphorylated | NO: 22) | | |
| | | at | | | |
| | | serine | | | |
| | | 278 | | | |
| P27361/ | Entry version 235 | Mitogen-activated | IADPEHD | MAPK3 | Sensitive |
| MK03_HUMAN | (2 Jun. 2021) | protein | HTGFLTE | (ERK1) | |
| | | kinase | YVATR | at | |
| | | 3 | (SEQ ID | T207 | |
| | | phosphorylated | NO: 23) | | |
| | | at | | | |
| | | threonine | | | |
| | | 207 | | | |
| Q16512/ | Entry version 230 | Serine/threonine- | SSRDPPS | PKN1 | Sensitive |
| PKN1_HUMAN | (2 Jun. 2021) | protein | SPSSLSS | at | |
| | | kinase | PIQESTA | S584 | |
| | | N1 | PELPSET | | |
| | | phosphorylated | QETPGPA | | |
| | | at | LCSPLR | | |
| | | serine | (SEQ ID | | |
| | | 584 | NO: 24) | | |
| P35236/ | Entry version 194 | Tyrosine-protein | IPSNFVS | PTPN7 | Sensitive |
| PTN7_HUMAN | (2 Jun. 2021) | phosphatase | PEDLDIP | at | |
| | | non-receptor | GHASK | S106 | |
| | | type | (SEQ ID | | |
| | | 7 | NO: 25) | | |
| | | phosphorylated | | | |
| | | at | | | |
| | | serine | | | |
| | | 106 | | | |
| P35236/ | Entry version 194 | Tyrosine-protein | GGMIQTA | PTPN7 | Sensitive |
| PTN7_HUMAN | (2 Jun. 2021) | phosphatase | EQYQFLH | at | |
| | | non-receptor | HTLALYA | S359 | |
| | | type | GQLPEEP | | |
| | | 7 | SP | | |
| | | phosphorylated | (SEQ ID | | |
| | | at | NO: 26) | | |
| | | serine | | | |
| | | 359 | | | |
| P31749/ | Entry version 251 | RAC-alpha | YFDEEFT | AKT1 | Sensitive |
| AKT1_HUMAN | (2 Jun. 2021) | serine/threonine- | AQMITIT | at | |
| | | protein | PPDQDDS | Y437 | |
| | | kinase | MECVDSE | | |
| | | phosphorylated | R | | |
| | | at | (SEQ ID | | |
| | | tyrosine | NO: 27) | | |
| | | 437 | | | |
| P31749/ | Entry version 251 | RAC-alpha | YFDEEFT | AKT1 | Sensitive |
| AKT1_HUMAN | (2 Jun. 2021) | serine/threonine- | AQMITIT | at | |
| | | protein | PPDQDDS | S457 | |
| | | kinase | MECVDSE | | |
| | | phosphorylated | R | | |
| | | at | (SEQ ID | | |
| | | serine | NO: 28) | | |
| | | 457 | | | |
| P31751/ | Entry version 224 | RAC-beta | YFDDEFT | AKT2 | Sensitive |
| AKT2_HUMAN | (2 Jun. 2021) | serine/threonine- | AQSITIT | at | |
| | | protein | PPDRYDS | S458 | |
| | | kinase | LGLLELD | | |
| | | phosphorylated | QR | | |
| | | at | (SEQ ID | | |
| | | serine | NO: 29) | | |
| | | 458 | | | |
| P31751/ | Entry version 224 | RAC-beta | YFDDEFT | AKT2 | Sensitive |
| AKT2_HUMAN | (2 Jun. 2021) | serine/threonine- | AQSITIT | at | |
| | | protein | PPDRYDS | Y438 | |
| | | kinase | LGLLELD | | |
| | | phosphorylated | QR | | |
| | | at | (SEQ ID | | |
| | | tyrosine | NO: 30) | | |
| | | 438 | | | |
| P27361/ | Entry version 235 | Mitogen-activated | IADPEHD | MAPK3 | Sensitive |
| MK03_HUMAN | (2 Jun. 2021) | protein | HTGFLTE | at | |
| | | kinase | YVATR | T202 | |
| | | 3 | (SEQ ID | | |
| | | phosphorylated | NO: 31) | | |
| | | at | | | |
| | | threonine | | | |
| | | 202 | | | |
| P27361/ | Entry version 235 | Mitogen-activated | IADPEHD | MAPK3 | Sensitive |
| MK03_HUMAN | (2 Jun. 2021) | protein | HTGFLTE | at | |
| | | kinase | YVATR | Y204 | |
| | | 3 | (SEQ ID | | |
| | | phosphorylated | NO: 32) | | |
| | | at | | | |
| | | tyrosine | | | |
| | | 204 | | | |
| Q13153/ | Entry version 229 | Serine/threonine- | DVATSPI | PAK1 | Sensitive |
| PAK1_HUMAN | (2 Jun. 2021) | protein | SPTENNT | at | |
| | | kinase | TPPDALT | T219 | |
| | | PAK | R | | |
| | | 1 | (SEQ ID | | |
| | | phosphorylated | NO: 33) | | |
| | | at | | | |
| | | serine | | | |
| | | 219 | | | |
| Q13153/ | Entry version 229 | Serine/threonine- | DVATSPI | PAK1 | Sensitive |
| PAK1_HUMAN | (2 Jun. 2021) | protein | SPTENNT | at | |
| | | kinase | TPPDALT | T229 | |
| | | PAK | R | | |
| | | 1 | (SEQ ID | | |
| | | phosphorylated | NO: 34) | | |
| | | at | | | |
| | | threonine | | | |
| | | 229 | | | |
| Q13153/ | Entry version 229 | Serine/threonine- | DVATSPI | PAK1 | Sensitive |
| PAK1_HUMAN | (2 Jun. 2021) | protein | SPTENNT | at | |
| | | kinase | TPPDALT | T230 | |
| | | PAK | R | | |
| | | 1 | (SEQ ID | | |
| | | phosphorylated | NO: 35) | | |
| | | at | | | |
| | | threonine | | | |
| | | 230 | | | |
| Q13541/ | Entry version 196 | Eukaryotic | DLPTIPG | EIF4EBP1 | Sensitive |
| 4EBP1_HUMAN | (2 Jun. 2021) | translation | VTSPSSD | at | |
| | | initiation | EPPMEAS | T77 | |
| | | factor | QSHLR | | |
| | | 4E-binding | (SEQ ID | | |
| | | protein | NO: 36) | | |
| | | 1 | | | |
| | | phosphorylated | | | |
| | | at | | | |
| | | threonine | | | |
| | | 77 | | | |
| Q02750/ | Entry version 226 | Dual | RSDAEEV | MAP2K1 | Sensitive |
| MP2K1_HUMAN | (2 Jun. 2021) | specificity | DFAGWLC | at | |
| | | mitogen- | STIGLNQ | S377 | |
| | | activated | PSTPTHA | | |
| | | protein | AGV | | |
| | | kinase | (SEQ ID | | |
| | | kinase | NO: 37) | | |
| | | 1 | | | |
| | | phosphorylated | | | |
| | | at | | | |
| | | serine | | | |
| | | 377 | | | |

TABLE 3

| Gene | pep_start | pep_end | Serine_residues | Threonine_residues | Tyrosine_residues |
|---|---|---|---|---|---|
| MAP3K2 | 329 | 348 | S331 | T337 | |
| | | | S344 | T339 | |
| | | | S347 | | |
| RPS6KA3 | 384 | 413 | S387 | T402 | |
| | | | S393 | | |
| | | | S397 | | |
| | | | S407 | | |
| ERBB2 | 1073 | 1096 | S1073 | | |
| | | | S1078 | | |
| | | | S1083 | | |
| MAP3K3 | 126 | 138 | S129 | | |
| | | | S130 | | |
| | | | S131 | | |
| | | | S134 | | |
| | | | S137 | | |
| EGFR | 987 | 999 | S991 | T993 | Y998 |
| | | | S995 | | |
| PHLPP1 | 1516 | 1529 | S1524 | | |
| | | | S1526 | | |
| MAP2K1 | 210 | 231 | S216 | T230 | |
| | | | S222 | | |
| | | | S226 | | |
| KRS1 | 190 | 217 | S192 | T198 | |
| | | | S194 | T200 | |
| | | | S197 | | |
| | | | S202 | | |
| | | | S205 | | |
| | | | S212 | | |
| | | | S213 | | |
| BRAF | 753 | 766 | | T753 | Y760 |
| MAP3K4 | 497 | 519 | S499 | T513 | |
| | | | S503 | | |
| | | | S512 | | |
| | | | S518 | | |
| MTOR | 2446 | 2470 | S2448 | T2446 | Y2449 |
| | | | S2450 | | |
| | | | S2454 | | |
| ARAF | 580 | 588 | S580 | | |
| | | | S582 | | |
| | | | S585 | | |
| RAF1 | 619 | 627 | S619 | | |
| | | | S621 | | |
| | | | S624 | | |
| PHLPP2 | 1208 | 1221 | S1210 | | |
| | | | S1213 | | |
| | | | S1220 | | |
| MAP3K20 | 646 | 655 | S648 | | |
| | | | S649 | | |
| | | | S654 | | |
| MAP2K2 | 5 | 39 | S23 | T13 | |
| | | | S26 | T17 | |
| | | | S30 | | |
| GSK3B | 206 | 220 | S215 | | Y216 |
| | | | S219 | | |
| GSK3A | 268 | 283 | S278 | | Y279 |
| | | | S282 | | |
| MAPK3 | 190 | 208 | | T198 | Y204 |
| | | | | T202 | |
| | | | | T207 | |
| PKN1 | 566 | 606 | S566 | T585 | |
| | | | S567 | T593 | |
| | | | S572 | T596 | |
| | | | S573 | | |
| | | | S575 | | |
| | | | S576 | | |
| | | | S578 | | |
| | | | S579 | | |
| | | | S584 | | |
| | | | S591 | | |
| | | | S603 | | |
| PTPN7 | 104 | 122 | S106 | | |
| | | | S110 | | |
| | | | S121 | | |
| PTPN7 | 331 | 360 | S359 | T336 | Y340 |
| | | | | T346 | Y350 |
| AKT1 | 437 | 465 | S457 | T443 | Y437 |
| | | | S463 | T448 | |
| | | | | T450 | |
| AKT2 | 438 | 467 | S447 | T444 | Y438 |
| | | | S458 | T449 | Y456 |
| | | | S460 | T451 | |
| PAK1 | 216 | 237 | S220 | T219 | |
| | | | S223 | T225 | |
| | | | | T229 | |
| | | | | T230 | |
| | | | | T236 | |
| EIF4EBP1 | 74 | 99 | S83 | T77 | |
| | | | S85 | T82 | |
| | | | S86 | | |
| | | | S94 | | |
| | | | S96 | | |
| MAP2K1 | 363 | 393 | S364 | T378 | |
| | | | S377 | T386 | |
| | | | S385 | T388 | |

TABLE 4

| Signatures | Signatures |
|---|---|
| FAB subtype | KDM1A_HUMAN expression |
| Karyotype | PK3CD_HUMAN expression |
| KRAS mutation | MP2K1_HUMAN expression |
| NPM1 mutation | MAP3K2 at S331 |
| DNMT3A mutation | MAP3K2 at T337 |
| NRAS mutation | MAP3K2 at T339 |
| IDH2 mutation | MAP3K2 at S344 |
| MTOR_HUMAN expression | MAP3K2 at S347 |
| MP2K2_HUMAN expression | RPS6KA3 at S387 |
| KS6B1_HUMAN expression | RPS6KA3 at S393 |
| MK01_HUMAN expression | RPS6KA3 at S397 |
| MP2K4_HUMAN expression | RPS6KA3 at T402 |
| KS6A1_HUMAN expression | RPS6KA3 at S407 |
| MP2K3_HUMAN expression | ERBB2 at S1073 |
| KS6A5_HUMAN expression | ERBB2 at S1078 |
| MP2K5_HUMAN expression | ERBB2 at S1083 |
| MAP3K3 at S130 | MAP3K3 at S129 |
| MAP3K3 at S131 | MAP3K20 at S649 |
| MAP3K3 at S134 | MAP3K20 at S654 |
| MAP3K3 at S137 | MAP2K2 (MEK2) at T13 |
| EGFR at S991 | MAP2K2 (MEK2) at T17 |
| EGFR at T993 | MAP2K2 (MEK2) at S23 |
| EGFR at S995 | MAP2K2 (MEK2) at S26 |
| EGFR at Y998 | MAP2K2 (MEK2) at S30 |
| PHLPP1 at S1524 | GSK3B at S215 |
| PHLPP1 at S1526 | GSK3B at Y217 |
| MAP2K1 at S216 | GSK3B at S219 |
| MAP2K1 at S222 | GSK3A at S278 |
| MAP2K1 at S226 | GSK3A at Y279 |
| MAP2K1 at T230 | GSK3A at S282 |
| KSR1 at S192 | MAPK3 (ERK1) at T198 |
| KSR1 at S194 | MAPK3 (ERK1) at T202 |
| KSR1 at S197 | MAPK3 (ERK1) at Y204 |
| KSR1 at T198 | MAPK3 (ERK1) at T207 |
| KSR1 at T200 | PKN1 at S566 |
| KSR1 at S202 | PKN1 at S567 |
| KSR1 at S205 | PKN1 at S572 |
| KSR1 at S212 | PKN1 at S573 |
| KSR1 at S213 | PKN1 at S575 |
| BRAF at T753 | PKN1 at S576 |
| BRAF at Y760 | PKN1 at S578 |
| MAP3K4 at S499 | PKN1 at S579 |
| MAP3K4 at S503 | PKN1 at S584 |
| MAP3K4 at S512 | PKN1 at T585 |
| MAP3K4 at T513 | PKN1 at S591 |
| MAP3K4 at S518 | PKN1 at T593 |
| ARAF at S580 | PKN1 at T596 |
| ARAF at S582 | PKN1 at S603 |
| ARAF at S585 | PTPN7 at S106 |
| RAF1 at S619 | PTPN7 at S110 |
| RAF1 at S621 | PTPN7 at S121 |
| RAF1 at S624 | PTPN7 at T336 |
| PHLPP2 at S1210 | PTPN7 at Y340 |
| PHLPP2 at S1213 | PTPN7 at T346 |
| PHLPP2 at S1220 | PTPN7 at Y350 |
| MAP3K20 at S648 | PTPN7 at S359 |
| AKT1 at T443 | AKT1 at Y437 |
| AKT1 at T448 | PAK1 at T229 |
| AKT1 at T450 | PAK1 at T230 |
| AKT1 at S457 | PAK1 at T236 |
| AKT1 at S463 | EIF4EBP1 at T77 |
| AKT2 at Y438 | EIF4EBP1 at T82 |
| AKT2 at T444 | EIF4EBP1 at S83 |
| AKT2 at S447 | EIF4EBP1 at S85 |
| AKT2 at T449 | EIF4EBP1 at S86 |
| AKT2 at T451 | EIF4EBP1 at S94 |
| AKT2 at Y456 | EIF4EBP1 at S96 |
| AKT2 at S458 | MAP2K1 at S364 |
| AKT2 at S560 | MAP2K1 at S377 |
| PAK1 at T219 | MAP2K1 at T378 |
| PAK1 at S220 | MAP2K1 at S385 |
| PAK1 at S223 | MAP2K1 at T386 |
| PAK1 at T225 | MAP2K1 at T388 |

The terms “markers”, “biomarkers” and “signatures” are used interchangeably herein. The expression of the one or more proteins; the level of phosphorylation at the one or more phosphorylation sites; the mutation of, or chromosomal rearrangement involving the genes and/or the classification by the French-American-British (FAB) AML classification scheme may be termed “markers”, “biomarkers” or “signatures” herein. As used herein, the term “one or more” embraces any integer from one up to and including the full number of biomarkers referenced. For example “one or more” may refer to any one, or two, or three, or four, or five, or six, or seven, or eight, or nine, or 10, or 11, or 12, or 13, or 14, or 15, or 16, or 17, or 18, or 19, or 20, or 21, or 22, or 23, or 24, or 25, or 26, or 27, or 28, or 29, or 30, or 31, or 32, or 33, or 34, or 35, or 36, or 37, or 38, or 39, or 40, or 41, or 42, or 43, or 44, or 45, or 46, or 47 or 48 or 49 or 50, or any integer between 51 and 146 inclusive, or all of the biomarkers referenced.

The biomarkers may comprise Serine/threonine-protein kinase PAK 1 phosphorylated at serine 219.

The biomarkers may comprise Glycogen synthase kinase-3 beta phosphorylated at serine 215 and/or Glycogen synthase kinase-3 alpha phosphorylated at serine 278.

The biomarkers may comprise KDM1A/LSD1 expression.

The biomarkers may comprise Ribosomal protein S6 kinase alpha-5 expression.

The biomarkers described herein further include mutation of, or chromosomal rearrangement involving, one or more of the following genes: KMT2A, NPM1, KRAS, NRAS, DNMT3A, IDH2, TP53, TET2, FLT3 and/or STAG2. The biomarkers described herein further include mutation of, or chromosomal rearrangement involving, one or more of the following genes: KMT2A, NPM1, KRAS, NRAS and/or DNMT3A. The mutation may be any mutation associated with cancer, for example AML. The mutation may be a point mutation, a deletion or an insertion. The biomarkers may comprise one or more KRAS mutation.

The KMT2A mutation or chromosomal rearrangement may be a point mutation or a rearrangement. The KMT2A mutation or chromosomal rearrangement may be one or more mutation or chromosomal rearrangement selected from the group consisting of a KMT2A-MLLT10 Rearrangement, a KMT2A-MLLT6 Rearrangement, a KMT2A-AFDN Rearrangement and a KMT2A-R2477Q Point mutation. The one or more KMT2A mutation or chromosomal rearrangement may be associated with cells sensitive to sequential treatment. The KMT2A-MLLT10 Rearrangement, the KMT2A-MLLT6 Rearrangement, the KMT2A-AFDN Rearrangement and the KMT2A-R2477Q Point mutation combined may be associated with cells sensitive to sequential treatment.

The KRAS mutation or chromosomal rearrangement may be a point mutation. The KRAS mutation may be one or more point mutation selected from the group consisting of a KRAS-G12P Point mutation, a KRAS-G12A Point mutation and a KRAS-Q61R Point mutation. The one or more KRAS point mutation may be associated with cells sensitive to sequential treatment. The KRAS-G12A Point mutation and the KRAS-Q61R Point mutation combined may be associated with cells sensitive to sequential treatment.

The NRAS mutation or chromosomal rearrangement may be a point mutation. The NRAS mutation may be one or more point mutation selected from the group consisting of a NRAS-G12D Point mutation, a NRAS-G13D Point mutation and a NRAS-Q61P Point mutation. The one or more NRAS point mutation may be associated with cells resistant to sequential treatment. The NRAS-G13D Point mutation and the NRAS-Q61 P Point mutation combined may be associated with cells resistant to sequential treatment.

The NPM1 mutation or chromosomal rearrangement may be an insertion. The NPM1 mutation may be one or more insertion selected from the group consisting of a NPM1-L287 Insertion (TCTG), a NPM1-L287 Insertion (TGCA) and a NPM1-W288 Insertion (GCTT). The one or more NPM1 mutation may be associated with cells resistant to sequential treatment. The NPM1-L287 Insertion (TCTG), the NPM1-L287 Insertion (TGCA) and the NPM1-W288 Insertion (GCTT) combined may be associated with cells resistant to sequential treatment.

The DNMT3A mutation or chromosomal rearrangement may be an insertion. The DNMT3A mutation may be one or more point mutation or splice donor variant selected from the group consisting of a DNMT3A-R882H Point mutation and a DNMT3A Splice donor variant. The one or more DNMT3A mutation may be associated with cells resistant to sequential treatment. The DNMT3A-R882H Point mutation and the DNMT3A Splice donor variant combined may be associated with cells resistant to sequential treatment.

The IDH2 mutation or chromosomal rearrangement may be a point mutation. The IDH2 point mutation may be a IDH2-R140Q Point mutation.

The TP53 mutation or chromosomal rearrangement may be a point mutation. The TP53 mutation may be one or more point mutation selected from the group consisting of a TP53-C106Y Point mutation and a TP53-A159P Point mutation.

The TET2 mutation or chromosomal rearrangement may be a point mutation or an insertion. The TET2 mutation may be one or more point mutation or insertion selected from the group consisting of a TET2-C1263Y Point mutation, a TET2-D1113 Insertion (AT) and a TET2-H924R Point mutation.

The FLT3 mutation or chromosomal rearrangement may be a point mutation or an insertion. The FLT3 mutation may be one or more point mutation or insertion selected from the group consisting of a FLT3-D835Y Point mutation, a FLT3-D835H Point mutation and a FLT3-internal tandem duplication (ITD) Insertion. The FLT3 mutation or chromosomal rearrangement may be a FLT3 internal tandem duplication (ITD).

The STAG2 mutation or chromosomal rearrangement may be a deletion. The STAG2 mutation may be a STAG2-M930 Deletion (T).

The biomarkers described herein further include classification as M5 by the French-American-British (FAB) AML classification scheme. FAB M5 subtype acute myeloid leukaemia may be associated with cells sensitive to sequential treatment.

The biomarkers may be any combination of biomarkers selected from those illustrated in FIG. 3 and/or FIG. 4.

The biomarkers may be any combination of biomarkers selected from those illustrated in FIG. 5. Combinations comprising markers of high ranked importance are explicitly contemplated herein. High ranked importance may be for AML cell lines (FIG. 5C), for primary AML cases (FIG. 5D) or both.

High ranked importance may refer to greater than 90 importance (RPS6KA3.T391 and/or PAK1.T219); greater than 75 ranked importance (RPS6KA3.T391 and/or KSR1.S202; and/or PAK1.T219 and/or GSK3B.S215/.S278); greater than 50 ranked importance; greater than 25 ranked importance; or greater than 0 ranked importance with reference to FIG. 5C and/or D.

The biomarkers may be selected from the group illustrated in FIG. 50, which are also shown in Table 5 below and are referred to herein interchangeably by their “signature” and “full biomarker name” where applicable.

TABLE 5

| Signature | Full biomarker name |
|---|---|
| PAK1.T219 | Serine/threonine-protein kinase PAK 1 phosphorylated at serine 219 |
| GSK3B.S215/.S278 | Glycogen synthase kinase-3 beta phosphorylated at serine 215 and/or |
| | Glycogen synthase kinase-3 alpha phosphorylated at serine 278 |
| KDM1A_HUMAN expression | Lysine-specific histone demethylase |
| MP2K5_HUMAN expression | Dual specificity mitogen-activated protein kinase kinase 2 |
| MAPK3.T202/.Y204 | Mitogen-activated protein kinase 3 phosphorylated at threonine 202 |
| MAPK3.Y204 | Mitogen-activated protein kinase 3 phosphorylated at tyrosine 204 |
| IDH2 mutation | IDH2- R140Q Point mutation |
| PTPN7.S106 | Tyrosine-protein phosphatase non-receptor type 7 phosphorylated at serine 106 |
| AKT1.Y437 | RAC-alpha serine/threonine-protein kinase phosphorylated at tyrosine 437 |
| EIF4EBP1.T77 | Eukaryotic translation initiation factor 4E-binding protein 1 phosphorylated |
| | at threonine 77 |
| KRAS mutation | KRAS-G12A Point mutation and/or KRAS-Q61R Point mutation |
| PAK1.T229/.T230 | Serine/threonine-protein kinase PAK 1 phosphorylated at threonine 229 |
| | And/or Serine/threonine-protein kinase PAK 1 phosphorylated at threonine 230 |
| PK3CD_HUMAN expression | Phosphatidylinositol 4,5-bisphosphate 3-kinase catalytic subunit delta isoform |
| TP53 mutation | TP53- C106Y Point mutation and/or TP53- A159P Point mutation |
| AKT1.S457 | RAC-alpha serine/threonine-protein kinase phosphorylated at serine 457 |
| KS6A5_HUMAN expression | Ribosomal protein S6 kinase alpha-5 |
| AKT2.S458 | RAC-beta serine/threonine-protein kinase phosphorylated at serine 458 |
| PKN1.S584 | Serine/threonine-protein kinase N1 phosphorylated at serine 584 |
| PTPN7.S359 | Tyrosine-protein phosphatase non-receptor type 7 phosphorylated at serine 359 |
| MAPK3.T207 | Mitogen-activated protein kinase 3 phosphorylated at threonine 207 |
| MP2K1_HUMAN expression | Dual specificity mitogen-activated protein kinase kinase 1 |
| DNMT3A mutation | DNMT3A-R882H Point mutation and/or DNMT3A Splice donor variant |
| TET2 mutation | TET2- C1263Y Point mutation, TET2-D1113 Insertion (AT) and/or TET2-H924R |
| | Point mutation |
| FLT3.ITD mutation | FLT3-D835Y Point mutation, FLT3-D835H Point mutation and/or FLT3- internal |
| | tandem duplication (ITD) Insertion |
| STAG2 mutation | STAG2-M930 Deletion |
| NPM1 mutation | NPM1-L287 Insertion (TCTG), NPM1-L287 Insertion (TGCA) and/or NPM1- |
| | W288 Insertion (GCTT) |
| MP2K3_HUMAN expression | Dual specificity mitogen-activated protein kinase kinase 3 |
| AKT2.Y438 | RAC-beta serine/threonine-protein kinase phosphorylated at tyrosine 438 |
| NRAS mutation | NRAS-G13D Point mutation and/or NRAS-Q61P Point mutation |
| KMTA2 rearranged | KMT2A-MLLT10 Rearrangement, KMT2A-MLLT6 Rearrangement, KMT2A-AFDN |
| karyotype | Rearrangement and/or KMT2A-R2477Q Point mutation |
| KS6A1_HUMAN expression | Ribosomal protein S6 kinase alpha-1 |
| MP2K4_HUMAN expression. | Dual specificity mitogen-activated protein kinase kinase 4 |

The “one or more” biomarker may be any one, or two, or three, or four, or five, or six, or seven, or eight, or nine, or 10, or 11, or 12, or 13, or 14, or 15, or 16, or 17, or 18, or 19, or 20, or 21, or 22, or 23, or 24, or 25, or 26, or 27, or 28, or 29, or 30, or 31, or 32, or 33, or 34 or all of the biomarkers referenced in Table 5.

The biomarkers may be selected from any group defined herein, optionally omitting any mutation or chromosomal rearrangement. Accordingly, with reference to the markers of Table 5, the biomarkers may be selected from the group shown in Table 6 below.

TABLE 6

| Signature | Full biomarker name |
|---|---|
| PAK1.T219 | Serine/threonine-protein kinase PAK 1 phosphorylated at serine 219 |
| GSK3B.S215/.S278 | Glycogen synthase kinase-3 beta phosphorylated at serine 215 and/or |
| | Glycogen synthase kinase-3 alpha phosphorylated at serine 278 |
| KDM1A_HUMAN expression | Lysine-specific histone demethylase |
| MP2K5_HUMAN expression | Dual specificity mitogen-activated protein kinase kinase 2 |
| MAPK3.T202/.Y204 | Mitogen-activated protein kinase 3 phosphorylated at threonine 202 |
| MAPK3.Y204 | Mitogen-activated protein kinase 3 phosphorylated at tyrosine 204 |
| PTPN7.S106 | Tyrosine-protein phosphatase non-receptor type 7 phosphorylated at serine 106 |
| AKT1.Y437 | RAC-alpha serine/threonine-protein kinase phosphorylated at tyrosine 437 |
| EIF4EBP1.T77 | Eukaryotic translation initiation factor 4E-binding protein 1 phosphorylated |
| | at threonine 77 |
| PAK1.T229/.T230 | Serine/threonine-protein kinase PAK 1 phosphorylated at threonine 229 |
| | And/or Serine/threonine-protein kinase PAK 1 phosphorylated at threonine 230 |
| PK3CD_HUMAN expression | Phosphatidylinositol 4,5-bisphosphate 3-kinase catalytic subunit delta isoform |
| AKT1.S457 | RAC-alpha serine/threonine-protein kinase phosphorylated at serine 457 |
| KS6A5_HUMAN expression | Ribosomal protein S6 kinase alpha-5 |
| AKT2.S458 | RAC-beta serine/threonine-protein kinase phosphorylated at serine 458 |
| PKN1.S584 | Serine/threonine-protein kinase N1 phosphorylated at serine 584 |
| PTPN7.S359 | Tyrosine-protein phosphatase non-receptor type 7 phosphorylated at serine 359 |
| MAPK3.T207 | Mitogen-activated protein kinase 3 phosphorylated at threonine 207 |
| MP2K1_HUMAN expression | Dual specificity mitogen-activated protein kinase kinase 1 |
| MP2K3_HUMAN expression | Dual specificity mitogen-activated protein kinase kinase 3 |
| AKT2.Y438 | RAC-beta serine/threonine-protein kinase phosphorylated at tyrosine 438 |
| KS6A1_HUMAN expression | Ribosomal protein S6 kinase alpha-1 |
| MP2K4_HUMAN expression. | Dual specificity mitogen-activated protein kinase kinase 4 |

The “one or more” biomarker may be any one, or two, or three, or four, or five, or six, or seven, or eight, or nine, or 10, or 11, or 12, or 13, or 14, or 15, or 16, or 17, or 18, or 19, or 20, or 21, or 22, or 23, or 24, or all of the biomarkers referenced in Table 6.

When it is predicted the cancer in the patient may be effectively treated with an epigenetic agent which inhibits a chromatin-modifying enzyme and/or which promotes cellular differentiation, followed by treatment with a kinase inhibitor, the patient may be said to have a sequential-treatment-responsive phenotype. Such a patient may also be said to be “sensitive” to sequential treatment. As used herein the term “sequential-treatment-responsive phenotype” and “sequential-treatment-responder phenotype” are used interchangeably. The sequential-treatment-responsive phenotype may alternatively be termed a sequential-treatment-responsive signature. The sequential-treatment-responsive phenotype may be a proteomic and/or phosphoproteomic phenotype. The patient may therefore be said to have a sequential-treatment-responsive proteomic phenotype and/or a sequential-treatment-responsive phosphoproteomic phenotype. The terms “phenotype” and “signature” may be used interchangeably. The patient may be said to have a sequential-treatment-responsive proteomic signature and/or a sequential-treatment-responsive phosphoproteomic signature. The proteomic phenotype may be defined by the level of the one or more proteins in the sample. The phosphoproteomic phenotype may be defined by the level of phosphorylation at the one or more phosphorylation sites in the sample. The sequential-treatment-responsive proteomic phenotype and/or sequential-treatment-responsive phosphoproteomic phenotype may therefore be determined in a sample detecting, in a sample of cancer cells obtained from the subject, the presence and/or level of one or more of the markers disclosed herein in accordance with the sixth aspect. The sequential-treatment-responsive proteomic phenotype may be determined by performing a proteomic assay on the sample from the patient. The proteomic assay may comprise determining the level of one or more of the proteins referred to herein. The sequential-treatment-responsive phosphoproteomic phenotype may be determined by performing a phosphoproteomic assay on the sample from the patient. The phosphoproteomic assay may comprise determining the level of phosphorylation at one or more phosphorylation sites referred to herein. Conversely, when it is predicted the cancer in the patient may not be effectively treated with an epigenetic agent which inhibits a chromatin-modifying enzyme and/or which promotes cellular differentiation, followed by treatment with a kinase inhibitor, the patient may be said to have a sequential-treatment-non-responsive phenotype or a sequential-treatment-resistant-phenotype. Such a patient may also be said to be “non-responsive” or “resistant” to sequential treatment. As used herein the term “sequential-treatment-non-responsive phenotype” and “sequential-treatment-non-responder phenotype” are used interchangeably. The sequential-treatment-non-responsive phenotype may alternatively be termed a sequential-treatment-non-responsive signature. The sequential-treatment-non-responsive phenotype may be a proteomic and/or phosphoproteomic phenotype. The patient may therefore be said to have a sequential-treatment-non-responsive proteomic phenotype and/or a sequential-treatment-non-responsive phosphoproteomic phenotype. The patient may be said to have a sequential-treatment-non-responsive proteomic signature and/or a sequential-treatment-non-responsive phosphoproteomic signature. The sequential-treatment-non-responsive proteomic phenotype and/or sequential-treatment-non-responsive phosphoproteomic phenotype may therefore be determined in a sample detecting, in a sample of cancer cells obtained from the subject, the presence and/or level of one or more of the markers disclosed herein in accordance with the sixth aspect. The sequential-treatment-non-responsive proteomic phenotype may be determined by performing a proteomic assay on the sample from the patient. The proteomic assay may comprise determining the level of one or more of the proteins referred to herein. The sequential-treatment-non-responsive phosphoproteomic phenotype may be determined by performing a phosphoproteomic assay on the sample from the patient. The phosphoproteomic assay may comprise determining the level of phosphorylation at one or more phosphorylation sites referred to herein.

As used herein, references to the level of the one or more proteins may refer to the expression level of the one or more proteins and vice versa; the terms are used interchangeably. References to expression of one or more proteins, at a “high level” (or a level that is high), as used here and elsewhere in the specification, denote a level of expression which is higher than the average level of expression of the relevant proteins. References to a “low level” of expression (or a level that is low) similarly denote a level of expression which is the same as or less than the average level of expression of the proteins. The average level of expression of the proteins is a standardised value which may be determined by reference to an average calculated across a plurality of samples, or by reference to the level of expression of the proteins in undifferentiated myeloblasts or other healthy cell types, which may be established either by laboratory analysis according to methods well known in the art (including LC-MS/MS), or by reference to information available in the art. Thus, for example, the average level of expression of the proteins may be determined by establishing the range of expression levels of the proteins in cell samples obtained from a large number of AML patients, and calculating the mean level of expression across the samples. A “high level” of expression is a level of expression which is higher than the calculated median or mean or upper quartile or threshold level. A “low level” of expression is a level of expression which is lower than the calculated median or mean or lower quartile or threshold level. A level of expression which is “not high” is a level of expression which is not higher than the calculated median or mean or upper quartile or threshold level; for example, the level of expression may be about or lower than the calculated median or mean or lower quartile or threshold level.

References to phosphorylation at a “high level” (or a level that is high), as used here and elsewhere in the specification, denote a level of phosphorylation which is higher than the average phosphorylation of the relevant protein or at the relevant phosphorylation site. References to a “low level” of phosphorylation similarly denote a level of phosphorylation which is the same as or less than the average phosphorylation of the relevant protein or at the relevant phosphorylation site. The average phosphorylation of the relevant protein or the relevant phosphorylation site is a standardised value which may be determined by reference to an average calculated across a plurality of samples, or by reference to the phosphorylation state of the relevant protein or the relevant phosphorylation site in undifferentiated myeloblasts or other healthy cell types, which may be established either by laboratory analysis according to methods well known in the art (including LC-MS/MS), or by reference to information available in the art. Thus, for example, the average level of phosphorylation at a particular phosphorylation site may be determined by establishing the range of phosphorylation at that site in cell samples obtained from a large number of AML patients, and calculating the mean phosphorylation across the samples. A “high level” of phosphorylation at that site is a level of phosphorylation which is higher than the calculated median or mean or upper quartile or threshold level. A “low level” of phosphorylation at that site is a level of phosphorylation which is lower than the calculated median or mean or lower quartile or threshold level. A level of phosphorylation which is “not high” is a level of phosphorylation which is not higher than the calculated median or mean or upper quartile or threshold level; for example, the level of phosphorylation may be about or lower than the calculated median or mean or lower quartile or threshold level.

The quantity of the one or more markers may exceed a threshold level per marker. The thresholds may be determined by measuring these sites across a population of cancer patients and then stablishing a cut-off value (e.g. the median value across patients) that is indicative of response. Alternatively, the levels may be determined by predictive modelling with a machine/statistical learning method. The biomarkers may predict that the cancer in the patient may be effectively treated with sequential-treatment when the level of the one or more biomarker is high. In other words, the one or more biomarker may be increased in patients with a sequential-treatment-responsive phenotype. Conversely, the one or more biomarker may be decreased in patients with a sequential-treatment-non-responsive phenotype. Markers said to be at a “high level” may be associated with sensitivity to sequential treatment.

Markers (i) may comprise a high level of phosphorylation at one or more phosphorylation sites selected from the group consisting of:
Serine/threonine-protein kinase PAK 1 phosphorylated at serine 219;
Mitogen-activated protein kinase kinase kinase 2 phosphorylated at threonine 339;
Ribosomal protein S6 kinase alpha-3 phosphorylated at threonine 391;
Receptor tyrosine-protein kinase erbB-2 phosphorylated at serine 1073;
Mitogen-activated protein kinase kinase kinase 3 phosphorylated at serine 129;
Epidermal growth factor receptor phosphorylated at serine 991;
PH domain leucine-rich repeat-containing protein phosphatase 1 phosphorylated at serine 1524;
Dual specificity mitogen-activated protein kinase kinase 1 phosphorylated at serine 222;
Dual specificity mitogen-activated protein kinase kinase 1 phosphorylated at serine 226;
Kinase suppressor of Ras 1 phosphorylated at serine 202;
Dual specificity mitogen-activated protein kinase kinase 1 phosphorylated at serine 230;
Dual specificity mitogen-activated protein kinase kinase 2 phosphorylated at threonine 13;
Mitogen-activated protein kinase 3 phosphorylated at threonine 207;
Serine/threonine-protein kinase N1 phosphorylated at serine 584;
Tyrosine-protein phosphatase non-receptor type 7 phosphorylated at serine 106;
Tyrosine-protein phosphatase non-receptor type 7 phosphorylated at serine 359;
RAC-alpha serine/threonine-protein kinase phosphorylated at tyrosine 437;
RAC-alpha serine/threonine-protein kinase phosphorylated at serine 457;
RAC-beta serine/threonine-protein kinase phosphorylated at serine 458;
RAC-beta serine/threonine-protein kinase phosphorylated at tyrosine 438;
Mitogen-activated protein kinase 3 phosphorylated at threonine 202;
Mitogen-activated protein kinase 3 phosphorylated at tyrosine 204;
Serine/threonine-protein kinase PAK 1 phosphorylated at threonine 229;
Serine/threonine-protein kinase PAK 1 phosphorylated at threonine 230;
Eukaryotic translation initiation factor 4E-binding protein 1 phosphorylated at threonine 77;
and/or
Dual specificity mitogen-activated protein kinase kinase 1 phosphorylated at serine 377.

Markers (i) may comprise a high level of phosphorylation at one or more phosphorylation sites selected from the group consisting of:
Serine/threonine-protein kinase PAK 1 phosphorylated at serine 219;
Mitogen-activated protein kinase 3 phosphorylated at threonine 207;
Serine/threonine-protein kinase N1 phosphorylated at serine 584;
Tyrosine-protein phosphatase non-receptor type 7 phosphorylated at serine 106;
Tyrosine-protein phosphatase non-receptor type 7 phosphorylated at serine 359;
RAC-alpha serine/threonine-protein kinase phosphorylated at tyrosine 437;
RAC-alpha serine/threonine-protein kinase phosphorylated at serine 457;
RAC-beta serine/threonine-protein kinase phosphorylated at serine 458;
RAC-beta serine/threonine-protein kinase phosphorylated at tyrosine 438;
Mitogen-activated protein kinase 3 phosphorylated at threonine 202;
Mitogen-activated protein kinase 3 phosphorylated at tyrosine 204;
Serine/threonine-protein kinase PAK 1 phosphorylated at threonine 229;
Serine/threonine-protein kinase PAK 1 phosphorylated at threonine 230; and/or
Eukaryotic translation initiation factor 4E-binding protein 1 phosphorylated at threonine 77.

Markers (i) may comprise a high level of phosphorylation of Serine/threonine-protein kinase PAK 1 phosphorylated at serine 219.

Markers (ii) may comprise a high level of one or more proteins selected from the group consisting of:
Lysine-specific histone demethylase;
Dual specificity mitogen-activated protein kinase kinase 2;
Ribosomal protein S6 kinase beta-1;
Mitogen-activated protein kinase 1;
Dual specificity mitogen-activated protein kinase kinase 4;
Ribosomal protein S6 kinase alpha-1;
Dual specificity mitogen-activated protein kinase kinase 3;
Phosphatidylinositol 4,5-bisphosphate 3-kinase catalytic subunit delta isoform; and/or
Dual specificity mitogen-activated protein kinase kinase 1.

Markers (ii) may comprise a high level of one or more proteins selected from the group consisting of:
Lysine-specific histone demethylase;
Dual specificity mitogen-activated protein kinase kinase 2;
Dual specificity mitogen-activated protein kinase kinase 4;
Ribosomal protein S6 kinase alpha-1;
Dual specificity mitogen-activated protein kinase kinase 3;
Phosphatidylinositol 4,5-bisphosphate 3-kinase catalytic subunit delta isoform; and/or
Dual specificity mitogen-activated protein kinase kinase 1;

Markers (ii) may comprise a high level of Lysine-specific histone demethylase.

The quantity of the one or more markers may be below a threshold level per marker. The thresholds may be determined by measuring these sites across a population of cancer patients and then stablishing a cut-off value (e.g. the median value across patients) that is indicative of response. Alternatively, the levels may be determined by predictive modelling with a machine/statistical learning method. The biomarkers may predict that the cancer in the patient may be effectively treated with sequential-treatment when the level of the one or more biomarker is low. In other words, the one or more biomarker may be decreased in patients with a sequential-treatment-responsive phenotype. Conversely, the one or more biomarker may be increased in patients with a sequential-treatment-non-responsive phenotype. Markers said to be at a “low level” or “or not a high level” may be associated with resistance to sequential treatment.

Markers (i) may comprise a low level or not a high level of phosphorylation at one or more phosphorylation sites selected from the group consisting of:
Glycogen synthase kinase-3 beta phosphorylated at serine 215;
Glycogen synthase kinase-3 alpha phosphorylated at serine 278;
Serine/threonine-protein kinase B-raf phosphorylated at threonine 753;
Mitogen-activated protein kinase kinase kinase 4 phosphorylated at serine 499;
Serine/threonine-protein kinase mTOR phosphorylated at threonine 2446;
Serine/threonine-protein kinase mTOR phosphorylated at serine 2448;
Serine/threonine-protein kinase A-Raf phosphorylated at serine 580;
RAF proto-oncogene serine/threonine-protein kinase phosphorylated at serine 619;
PH domain leucine-rich repeat-containing protein phosphatase 2 phosphorylated at serine 1210;
Mitogen-activated protein kinase kinase kinase 20 at serine 648; and/or
Mitogen-activated protein kinase kinase kinase 20 at serine 649;

Markers (i) may comprise a low level or not a high level of phosphorylation at one or more phosphorylation sites selected from the group consisting of:
Glycogen synthase kinase-3 beta phosphorylated at serine 215; and/or
Glycogen synthase kinase-3 alpha phosphorylated at serine 278.

Markers (i) may comprise a low level or not a high level of phosphorylation of Glycogen synthase kinase-3 beta phosphorylated at serine 215. Markers (i) may comprise a low level or not a high level of phosphorylation of Glycogen synthase kinase-3 alpha phosphorylated at serine 278.

Markers (ii) may comprise a low level or not a high level of one or more proteins selected from the group consisting of:
Ribosomal protein S6 kinase alpha-5;
Serine/threonine-protein kinase mTOR; and/or
Dual specificity mitogen-activated protein kinase kinase 5.

Markers (ii) comprise a low level or not a high level of Ribosomal protein S6 kinase alpha-5.

Any biomarker disclosed herein of which a low level may be used to predict that that the cancer in the patient may be effectively treated with sequential-treatment may alternatively be used to predict that that the cancer in the patient may not be effectively treated with sequential-treatment when the biomarker is at a high level. In other words, such biomarkers may be increased in patients with a sequential-treatment-non-responsive phenotype.

Any biomarker disclosed herein of which a low level may be used to predict that that the cancer in the patient may be effectively treated with sequential-treatment (in particular biomarkers shown herein to be increased in non-responders) may alternatively be used to predict that that the cancer in the patient may be effectively treated with sequential-treatment when the level of said biomarker is not high.

Any biomarker disclosed herein of which a high level may be used to predict that that the cancer in the patient may be effectively treated with sequential-treatment may alternatively be used to predict that that the cancer in the patient may not be effectively treated with sequential-treatment when the biomarker is at a low level. In other words, such biomarkers may be decreased in patients with a sequential-treatment-non-responsive phenotype.

The methods of the first and/or second aspects of the invention may comprise assaying a sample of cells from the cancer for the presence of one or more biomarkers described herein. Treatment may be based on the outcome of the assay. A high level of one or more biomarker increased in cells sensitive to sequential treatment and/or in patients with a sequential-treatment-responsive phenotype may result in selection of the patient for sequential treatment. A low level of one or more biomarker increased in cells resistant to sequential treatment and/or in patients with a sequential-treatment-non-responsive phenotype may result in selection of the patient for sequential treatment. Any suitable combination of the biomarkers described herein may be used to select a patient for sequential treatment. Conversely, a low level of one or more biomarker increased in cells sensitive to sequential treatment and/or in patients with a sequential-treatment-responsive phenotype may result in deselection of the patient for sequential treatment. A high level of one or more biomarker increased in cells resistant to sequential treatment and/or in patients with a sequential-treatment-non-responsive phenotype may result in deselection of the patient for sequential treatment. Any suitable combination of the biomarkers described herein may be used to deselect a patient for sequential treatment.

The methods of the first and/or second aspects of the invention may be alternatively described as a method for the treatment of cancer in a patient in need thereof comprising:
(1) assaying a sample of cells from the cancer for the presence of one or more biomarkers selected from the group consisting of:
   (i) phosphorylation at one or more of the following phosphorylation sites:
      Mitogen-activated protein kinase kinase kinase 2 phosphorylated at threonine 339;
      Ribosomal protein S6 kinase alpha-3 phosphorylated at threonine 391;
      Receptor tyrosine-protein kinase erbB-2 phosphorylated at serine 1073;
      Mitogen-activated protein kinase kinase kinase 3 phosphorylated at serine 129;
      Epidermal growth factor receptor phosphorylated at serine 991;
      PH domain leucine-rich repeat-containing protein phosphatase 1 phosphorylated at serine 1524;
      Dual specificity mitogen-activated protein kinase kinase 1 phosphorylated at serine 222;
      Dual specificity mitogen-activated protein kinase kinase 1 phosphorylated at serine 226;
      Kinase suppressor of Ras 1 phosphorylated at serine 202;
      Dual specificity mitogen-activated protein kinase kinase 1 phosphorylated at serine 230;
      Serine/threonine-protein kinase B-raf phosphorylated at threonine 753;
      Mitogen-activated protein kinase kinase kinase 4 phosphorylated at serine 499;
      Serine/threonine-protein kinase mTOR phosphorylated at threonine 2446;
      Serine/threonine-protein kinase mTOR phosphorylated at serine 2448;
      Serine/threonine-protein kinase A-Raf phosphorylated at serine 580;
      RAF proto-oncogene serine/threonine-protein kinase phosphorylated at serine 619;
      PH domain leucine-rich repeat-containing protein phosphatase 2 phosphorylated at serine 1210;
      Mitogen-activated protein kinase kinase kinase 20 at serine 648;
      Mitogen-activated protein kinase kinase kinase 20 at serine 649;
      Dual specificity mitogen-activated protein kinase kinase 2 phosphorylated at threonine 13;
      Glycogen synthase kinase-3 beta phosphorylated at serine 215;
      Glycogen synthase kinase-3 alpha phosphorylated at serine 278;
      Mitogen-activated protein kinase 3 phosphorylated at threonine 207;
      Serine/threonine-protein kinase N1 phosphorylated at serine 584;
      Tyrosine-protein phosphatase non-receptor type 7 phosphorylated at serine 359;
      RAC-alpha serine/threonine-protein kinase phosphorylated at serine 457;
      RAC-beta serine/threonine-protein kinase phosphorylated at serine 458;
      RAC-beta serine/threonine-protein kinase phosphorylated at tyrosine 438;
      Mitogen-activated protein kinase 3 phosphorylated at threonine 202;
      Mitogen-activated protein kinase 3 phosphorylated at tyrosine 204
      Serine/threonine-protein kinase PAK 1 phosphorylated at serine 219;
      Serine/threonine-protein kinase PAK 1 phosphorylated at threonine 229;
      Serine/threonine-protein kinase PAK 1 phosphorylated at threonine 230;
      Eukaryotic translation initiation factor 4E-binding protein 1 phosphorylated at threonine 77; and/or
      Dual specificity mitogen-activated protein kinase kinase 1 phosphorylated at serine 377; and/or
   (ii) expression of one or more of the following proteins:
      Lysine-specific histone demethylase;
      Serine/threonine-protein kinase mTOR;
      Dual specificity mitogen-activated protein kinase kinase 2;
      Ribosomal protein S6 kinase beta-1;
      Mitogen-activated protein kinase 1;
      Dual specificity mitogen-activated protein kinase kinase 4;
      Ribosomal protein S6 kinase alpha-1;
      Dual specificity mitogen-activated protein kinase kinase 3;
      Ribosomal protein S6 kinase alpha-5;
      Dual specificity mitogen-activated protein kinase kinase 5;
      Phosphatidylinositol 4,5-bisphosphate 3-kinase catalytic subunit delta isoform; and/or
      Dual specificity mitogen-activated protein kinase kinase 1; and/or
   (iii) mutation of, or chromosomal rearrangement involving, one or more of the following genes: KMT2A, NPM1, KRAS, NRAS, DNMT3A, IDH2, TP53, TET2, FLT3 and/or STAG2; and/or
   (iv) classified as M5 by the French-American-British (FAB) AML classification scheme;
(2) administering an epigenetic agent to the patient; and
(3) subsequently administering a protein kinase inhibitor to the patient.

The methods of the first and/or second aspects of the invention may comprise selecting the patient for treatment by assaying for the presence of one or more biomarkers described herein. The patient may have been selected for treatment by assaying for the presence of one or more biomarkers described herein. A high level of one or more biomarker increased in cells sensitive to sequential treatment and/or in patients with a sequential-treatment-responsive phenotype may select the patient for sequential treatment. A low level of one or more biomarker increased in cells resistant to sequential treatment and/or in patients with a sequential-treatment-non-responsive phenotype may select the patient for sequential treatment. Any suitable combination of the biomarkers described herein may be used to select a patient for sequential treatment. Conversely, a low level of one or more biomarker increased in cells sensitive to sequential treatment and/or in patients with a sequential-treatment-responsive phenotype may deselect the patient for sequential treatment. A high level of one or more biomarker increased in cells resistant to sequential treatment and/or in patients with a sequential-treatment-non-responsive phenotype may deselect the patient for sequential treatment. Any suitable combination of the biomarkers described herein may be used to deselect a patient for sequential treatment.

The methods of the first and/or second aspects of the invention may be alternatively described as a method for the treatment of cancer in a patient in need thereof, comprising
(1) administering an epigenetic agent to the patient; and
(2) subsequently administering a protein kinase inhibitor to the patient, wherein the patient is selected for treatment by assaying for the presence of one or more biomarkers selected from the group consisting of:
(i) phosphorylation at one or more of the following phosphorylation sites:
   Mitogen-activated protein kinase kinase kinase 2 phosphorylated at threonine 339;
   Ribosomal protein S6 kinase alpha-3 phosphorylated at threonine 391;
   Receptor tyrosine-protein kinase erbB-2 phosphorylated at serine 1073;
   Mitogen-activated protein kinase kinase kinase 3 phosphorylated at serine 129;
   Epidermal growth factor receptor phosphorylated at serine 991;
   PH domain leucine-rich repeat-containing protein phosphatase 1 phosphorylated at serine 1524;
   Dual specificity mitogen-activated protein kinase kinase 1 phosphorylated at serine 222;
   Dual specificity mitogen-activated protein kinase kinase 1 phosphorylated at serine 226;
   Kinase suppressor of Ras 1 phosphorylated at serine 202;
   Dual specificity mitogen-activated protein kinase kinase 1 phosphorylated at serine 230;
   Serine/threonine-protein kinase B-raf phosphorylated at threonine 753;
   Mitogen-activated protein kinase kinase kinase 4 phosphorylated at serine 499;
   Serine/threonine-protein kinase mTOR phosphorylated at threonine 2446;
   Serine/threonine-protein kinase mTOR phosphorylated at serine 2448;
   Serine/threonine-protein kinase A-Raf phosphorylated at serine 580;
   RAF proto-oncogene serine/threonine-protein kinase phosphorylated at serine 619;
   PH domain leucine-rich repeat-containing protein phosphatase 2 phosphorylated at serine 1210;
   Mitogen-activated protein kinase kinase kinase 20 at serine 648;
   Mitogen-activated protein kinase kinase kinase 20 at serine 649;
   Dual specificity mitogen-activated protein kinase kinase 2 phosphorylated at threonine 13;
   Glycogen synthase kinase-3 beta phosphorylated at serine 215;
   Glycogen synthase kinase-3 alpha phosphorylated at serine 278;
   Mitogen-activated protein kinase 3 phosphorylated at threonine 207;
   Serine/threonine-protein kinase N1 phosphorylated at serine 584;
   Tyrosine-protein phosphatase non-receptor type 7 phosphorylated at serine 359;
   RAC-alpha serine/threonine-protein kinase phosphorylated at serine 457;
   RAC-beta serine/threonine-protein kinase phosphorylated at serine 458;
   RAC-beta serine/threonine-protein kinase phosphorylated at tyrosine 438;
   Mitogen-activated protein kinase 3 phosphorylated at threonine 202;
   Mitogen-activated protein kinase 3 phosphorylated at tyrosine 204;
   Serine/threonine-protein kinase PAK 1 phosphorylated at serine 219;
   Serine/threonine-protein kinase PAK 1 phosphorylated at threonine 229;
   Serine/threonine-protein kinase PAK 1 phosphorylated at threonine 230;
   Eukaryotic translation initiation factor 4E-binding protein 1 phosphorylated at threonine 77; and/or
   Dual specificity mitogen-activated protein kinase kinase 1 phosphorylated at serine 377; and/or
(ii) expression of one or more of the following proteins:
   Lysine-specific histone demethylase;
   Serine/threonine-protein kinase mTOR;
   Dual specificity mitogen-activated protein kinase kinase 2;
   Ribosomal protein S6 kinase beta-1;
   Mitogen-activated protein kinase 1;
   Dual specificity mitogen-activated protein kinase kinase 4;
   Ribosomal protein S6 kinase alpha-1;
   Dual specificity mitogen-activated protein kinase kinase 3;
   Ribosomal protein S6 kinase alpha-5;
   Dual specificity mitogen-activated protein kinase kinase 5;
   Phosphatidylinositol 4,5-bisphosphate 3-kinase catalytic subunit delta isoform; and/or
   Dual specificity mitogen-activated protein kinase kinase 1; and/or
(iii) mutation of, or chromosomal rearrangement involving, one or more of the following genes: KMT2A, NPM1, KRAS, NRAS, DNMT3A, IDH2, TP53, TET2, FLT3 and/or STAG2; and/or
(iv) classified as M5 by the French-American-British (FAB) AML classification scheme.

The methods of the first and/or second aspects of the invention may comprise administering a protein kinase inhibitor to the patient wherein the cancer is characterised by the presence of one or more biomarkers described herein.

The methods of the first and/or second aspects of the invention may be alternatively described as a method for the treatment of cancer in a patient in need thereof comprising:
(1) administering an epigenetic agent to the patient; and
(2) subsequently administering a protein kinase inhibitor to the patient, wherein the cancer is characterised by the presence of one or more biomarkers selected from the group consisting of:
   (i) phosphorylation at one or more of the following phosphorylation sites:
      Mitogen-activated protein kinase kinase kinase 2 phosphorylated at threonine 339;
      Ribosomal protein S6 kinase alpha-3 phosphorylated at threonine 391;
      Receptor tyrosine-protein kinase erbB-2 phosphorylated at serine 1073;
      Mitogen-activated protein kinase kinase kinase 3 phosphorylated at serine 129;
      Epidermal growth factor receptor phosphorylated at serine 991;
      PH domain leucine-rich repeat-containing protein phosphatase 1 phosphorylated at serine 1524;
      Dual specificity mitogen-activated protein kinase kinase 1 phosphorylated at serine 222;
      Dual specificity mitogen-activated protein kinase kinase 1 phosphorylated at serine 226;
      Kinase suppressor of Ras 1 phosphorylated at serine 202;
      Dual specificity mitogen-activated protein kinase kinase 1 phosphorylated at serine 230;
      Serine/threonine-protein kinase B-raf phosphorylated at threonine 753;
      Mitogen-activated protein kinase kinase kinase 4 phosphorylated at serine 499;
      Serine/threonine-protein kinase mTOR phosphorylated at threonine 2446;
      Serine/threonine-protein kinase mTOR phosphorylated at serine 2448;
      Serine/threonine-protein kinase A-Raf phosphorylated at serine 580;
      RAF proto-oncogene serine/threonine-protein kinase phosphorylated at serine 619;
      PH domain leucine-rich repeat-containing protein phosphatase 2 phosphorylated at serine 1210;
      Mitogen-activated protein kinase kinase kinase 20 at serine 648;
      Mitogen-activated protein kinase kinase kinase 20 at serine 649;
      Dual specificity mitogen-activated protein kinase kinase 2 phosphorylated at threonine 13;
      Glycogen synthase kinase-3 beta phosphorylated at serine 215;
      Glycogen synthase kinase-3 alpha phosphorylated at serine 278;
      Mitogen-activated protein kinase 3 phosphorylated at threonine 207;
      Serine/threonine-protein kinase N1 phosphorylated at serine 584;
      Tyrosine-protein phosphatase non-receptor type 7 phosphorylated at serine 359;
      RAC-alpha serine/threonine-protein kinase phosphorylated at serine 457;
      RAC-beta serine/threonine-protein kinase phosphorylated at serine 458;
      RAC-beta serine/threonine-protein kinase phosphorylated at tyrosine 438;
      Mitogen-activated protein kinase 3 phosphorylated at threonine 202;
      Mitogen-activated protein kinase 3 phosphorylated at tyrosine 204;
      Serine/threonine-protein kinase PAK 1 phosphorylated at serine 219;
      Serine/threonine-protein kinase PAK 1 phosphorylated at threonine 229;
      Serine/threonine-protein kinase PAK 1 phosphorylated at threonine 230;
      Eukaryotic translation initiation factor 4E-binding protein 1 phosphorylated at threonine 77; and/or
      Dual specificity mitogen-activated protein kinase kinase 1 phosphorylated at serine 377; and/or
   (ii) expression of one or more of the following proteins:
      Lysine-specific histone demethylase;
      Serine/threonine-protein kinase mTOR;
      Dual specificity mitogen-activated protein kinase kinase 2;
      Ribosomal protein S6 kinase beta-1;
      Mitogen-activated protein kinase 1;
      Dual specificity mitogen-activated protein kinase kinase 4;
      Ribosomal protein S6 kinase alpha-1;
      Dual specificity mitogen-activated protein kinase kinase 3;
      Ribosomal protein S6 kinase alpha-5;
      Dual specificity mitogen-activated protein kinase kinase 5;
      Phosphatidylinositol 4,5-bisphosphate 3-kinase catalytic subunit delta isoform; and/or
      Dual specificity mitogen-activated protein kinase kinase 1; and/or
   (iii) mutation of, or chromosomal rearrangement involving, one or more of the following genes: KMT2A, NPM1, KRAS, NRAS, DNMT3A, IDH2, TP53, TET2, FLT3 and/or STAG2; and/or
   (iv) classified as M5 by the French-American-British (FAB) AML classification scheme;

Features described in connection with the first and/or second aspect are explicitly contemplated herein in combination with the features of the third and/or fourth aspects. For example, the third and/or fourth aspects of the invention may comprise selecting the patient for treatment by assaying for the presence of one or more biomarkers described herein. The patient may have been selected for treatment by assaying for the presence of one or more biomarkers described herein. The cancer may be characterised by the presence of one or more biomarkers.

The kit of the fifth aspect may alternatively be described as a kit comprising an epigenetic agent and a protein kinase inhibitor for simultaneous, separate or sequential use in the treatment of cancer in a patient, wherein the patient is screened for the presence of one or more biomarkers selected from the group consisting of:
(i) phosphorylation at one or more of the following phosphorylation sites:
   Mitogen-activated protein kinase kinase kinase 2 phosphorylated at threonine 339;
   Ribosomal protein S6 kinase alpha-3 phosphorylated at threonine 391;
   Receptor tyrosine-protein kinase erbB-2 phosphorylated at serine 1073;
   Mitogen-activated protein kinase kinase kinase 3 phosphorylated at serine 129;
   Epidermal growth factor receptor phosphorylated at serine 991;
   PH domain leucine-rich repeat-containing protein phosphatase 1 phosphorylated at serine 1524;
   Dual specificity mitogen-activated protein kinase kinase 1 phosphorylated at serine 222;
   Dual specificity mitogen-activated protein kinase kinase 1 phosphorylated at serine 226;
   Kinase suppressor of Ras 1 phosphorylated at serine 202;
   Dual specificity mitogen-activated protein kinase kinase 1 phosphorylated at serine 230;
   Serine/threonine-protein kinase B-raf phosphorylated at threonine 753;
   Mitogen-activated protein kinase kinase kinase 4 phosphorylated at serine 499;
   Serine/threonine-protein kinase mTOR phosphorylated at threonine 2446;
   Serine/threonine-protein kinase mTOR phosphorylated at serine 2448;
   Serine/threonine-protein kinase A-Raf phosphorylated at serine 580;
   RAF proto-oncogene serine/threonine-protein kinase phosphorylated at serine 619;
   PH domain leucine-rich repeat-containing protein phosphatase 2 phosphorylated at serine 1210;
   Mitogen-activated protein kinase kinase kinase 20 at serine 648;
   Mitogen-activated protein kinase kinase kinase 20 at serine 649;
   Dual specificity mitogen-activated protein kinase kinase 2 phosphorylated at threonine 13;
   Glycogen synthase kinase-3 beta phosphorylated at serine 215;
   Glycogen synthase kinase-3 alpha phosphorylated at serine 278;
   Mitogen-activated protein kinase 3 phosphorylated at threonine 207;
   Serine/threonine-protein kinase N1 phosphorylated at serine 584;
   Tyrosine-protein phosphatase non-receptor type 7 phosphorylated at serine 359;
   RAC-alpha serine/threonine-protein kinase phosphorylated at serine 457;
   RAC-beta serine/threonine-protein kinase phosphorylated at serine 458;
   RAC-beta serine/threonine-protein kinase phosphorylated at tyrosine 438;
   Mitogen-activated protein kinase 3 phosphorylated at threonine 202;
   Mitogen-activated protein kinase 3 phosphorylated at tyrosine 204;
   Serine/threonine-protein kinase PAK 1 phosphorylated at serine 219;
   Serine/threonine-protein kinase PAK 1 phosphorylated at threonine 229;
   Serine/threonine-protein kinase PAK 1 phosphorylated at threonine 230;
   Eukaryotic translation initiation factor 4E-binding protein 1 phosphorylated at threonine 77; and/or
   Dual specificity mitogen-activated protein kinase kinase 1 phosphorylated at serine 377; and/or
(ii) expression of one or more of the following proteins:
   Lysine-specific histone demethylase;
   Serine/threonine-protein kinase mTOR;
   Dual specificity mitogen-activated protein kinase kinase 2;
   Ribosomal protein S6 kinase beta-1;
   Mitogen-activated protein kinase 1;
   Dual specificity mitogen-activated protein kinase kinase 4;
   Ribosomal protein S6 kinase alpha-1;
   Dual specificity mitogen-activated protein kinase kinase 3;
   Ribosomal protein S6 kinase alpha-5;
   Dual specificity mitogen-activated protein kinase kinase 5;
   Phosphatidylinositol 4,5-bisphosphate 3-kinase catalytic subunit delta isoform; and/or
   Dual specificity mitogen-activated protein kinase kinase 1; and/or
(iii) mutation of, or chromosomal rearrangement involving, one or more of the following genes: KMT2A, NPM1, KRAS, NRAS, DNMT3A, IDH2, TP53, TET2, FLT3 and/or STAG2; and/or
(iv) classified as M5 by the French-American-British (FAB) AML classification scheme;

The kit of the fifth aspect may alternatively be described as a kit comprising an epigenetic agent and a protein kinase inhibitor for simultaneous, separate or sequential use in the treatment of cancer in a patient, wherein the cancer is characterised by one or more biomarkers selected from the group consisting of:
(i) phosphorylation at one or more of the following phosphorylation sites:
   Mitogen-activated protein kinase kinase kinase 2 phosphorylated at threonine 339;
   Ribosomal protein S6 kinase alpha-3 phosphorylated at threonine 391;
   Receptor tyrosine-protein kinase erbB-2 phosphorylated at serine 1073;
   Mitogen-activated protein kinase kinase kinase 3 phosphorylated at serine 129;
   Epidermal growth factor receptor phosphorylated at serine 991;
   PH domain leucine-rich repeat-containing protein phosphatase 1 phosphorylated at serine 1524;
   Dual specificity mitogen-activated protein kinase kinase 1 phosphorylated at serine 222;
   Dual specificity mitogen-activated protein kinase kinase 1 phosphorylated at serine 226;
   Kinase suppressor of Ras 1 phosphorylated at serine 202;
   Dual specificity mitogen-activated protein kinase kinase 1 phosphorylated at serine 230;
   Serine/threonine-protein kinase B-raf phosphorylated at threonine 753;
   Mitogen-activated protein kinase kinase kinase 4 phosphorylated at serine 499;
   Serine/threonine-protein kinase mTOR phosphorylated at threonine 2446;
   Serine/threonine-protein kinase mTOR phosphorylated at serine 2448;
   Serine/threonine-protein kinase A-Raf phosphorylated at serine 580;
   RAF proto-oncogene serine/threonine-protein kinase phosphorylated at serine 619;
   PH domain leucine-rich repeat-containing protein phosphatase 2 phosphorylated at serine 1210;
   Mitogen-activated protein kinase kinase kinase 20 at serine 648;
   Mitogen-activated protein kinase kinase kinase 20 at serine 649;
   Dual specificity mitogen-activated protein kinase kinase 2 phosphorylated at threonine 13;
   Glycogen synthase kinase-3 beta phosphorylated at serine 215;
   Glycogen synthase kinase-3 alpha phosphorylated at serine 278;
   Mitogen-activated protein kinase 3 phosphorylated at threonine 207;
   Serine/threonine-protein kinase N1 phosphorylated at serine 584;
   Tyrosine-protein phosphatase non-receptor type 7 phosphorylated at serine 359;
   RAC-alpha serine/threonine-protein kinase phosphorylated at serine 457;
   RAC-beta serine/threonine-protein kinase phosphorylated at serine 458;
   RAC-beta serine/threonine-protein kinase phosphorylated at tyrosine 438;
   Mitogen-activated protein kinase 3 phosphorylated at threonine 202;
   Mitogen-activated protein kinase 3 phosphorylated at tyrosine 204;
   Serine/threonine-protein kinase PAK 1 phosphorylated at serine 219;
   Serine/threonine-protein kinase PAK 1 phosphorylated at threonine 229;
   Serine/threonine-protein kinase PAK 1 phosphorylated at threonine 230;
   Eukaryotic translation initiation factor 4E-binding protein 1 phosphorylated at threonine 77; and/or
   Dual specificity mitogen-activated protein kinase kinase 1 phosphorylated at serine 377; and/or
(ii) expression of one or more of the following proteins:
   Lysine-specific histone demethylase;
   Serine/threonine-protein kinase mTOR;
   Dual specificity mitogen-activated protein kinase kinase 2;
   Ribosomal protein S6 kinase beta-1;
   Mitogen-activated protein kinase 1;
   Dual specificity mitogen-activated protein kinase kinase 4;
   Ribosomal protein S6 kinase alpha-1;
   Dual specificity mitogen-activated protein kinase kinase 3;
   Ribosomal protein S6 kinase alpha-5;
   Dual specificity mitogen-activated protein kinase kinase 5;
   Phosphatidylinositol 4,5-bisphosphate 3-kinase catalytic subunit delta isoform; and/or
   Dual specificity mitogen-activated protein kinase kinase 1; and/or
(iii) mutation of, or chromosomal rearrangement involving, one or more of the following genes: KMT2A, NPM1, KRAS, NRAS, DNMT3A, IDH2, TP53, TET2, FLT3 and/or STAG2; and/or
(iv) classified as M5 by the French-American-British (FAB) AML classification scheme.

According to a sixth aspect, the invention provides a method of selecting a subject with cancer for treatment with an epigenetic agent which inhibits a chromatin-modifying enzyme and/or which promotes cellular differentiation, followed by treatment with a kinase inhibitor, wherein the method comprises:
detecting, in a sample of cancer cells obtained from the subject, the presence and/or level of one or more of the following markers:
   (i) phosphorylation at one or more of the following phosphorylation sites:
      Mitogen-activated protein kinase kinase kinase 2 phosphorylated at threonine 339;
      Ribosomal protein S6 kinase alpha-3 phosphorylated at threonine 391;
      Receptor tyrosine-protein kinase erbB-2 phosphorylated at serine 1073;
      Mitogen-activated protein kinase kinase kinase 3 phosphorylated at serine 129;
      Epidermal growth factor receptor phosphorylated at serine 991;
      PH domain leucine-rich repeat-containing protein phosphatase 1 phosphorylated at serine 1524;
      Dual specificity mitogen-activated protein kinase kinase 1 phosphorylated at serine 222;
      Dual specificity mitogen-activated protein kinase kinase 1 phosphorylated at serine 226;
      Kinase suppressor of Ras 1 phosphorylated at serine 202;
      Dual specificity mitogen-activated protein kinase kinase 1 phosphorylated at serine 230;
      Serine/threonine-protein kinase B-raf phosphorylated at threonine 753;
      Mitogen-activated protein kinase kinase kinase 4 phosphorylated at serine 499;
      Serine/threonine-protein kinase mTOR phosphorylated at threonine 2446;
      Serine/threonine-protein kinase mTOR phosphorylated at serine 2448;
      Serine/threonine-protein kinase A-Raf phosphorylated at serine 580;
      RAF proto-oncogene serine/threonine-protein kinase phosphorylated at serine 619;
      PH domain leucine-rich repeat-containing protein phosphatase 2 phosphorylated at serine 1210;
      Mitogen-activated protein kinase kinase kinase 20 at serine 648;
      Mitogen-activated protein kinase kinase kinase 20 at serine 649;
      Dual specificity mitogen-activated protein kinase kinase 2 phosphorylated at threonine 13;
      Glycogen synthase kinase-3 beta phosphorylated at serine 215;
      Glycogen synthase kinase-3 alpha phosphorylated at serine 278;
      Mitogen-activated protein kinase 3 phosphorylated at threonine 207;
      Serine/threonine-protein kinase N1 phosphorylated at serine 584;
      Tyrosine-protein phosphatase non-receptor type 7 phosphorylated at serine 359;
      RAC-alpha serine/threonine-protein kinase phosphorylated at serine 457;
      RAC-beta serine/threonine-protein kinase phosphorylated at serine 458;
      RAC-beta serine/threonine-protein kinase phosphorylated at tyrosine 438;
      Mitogen-activated protein kinase 3 phosphorylated at threonine 202;
      Mitogen-activated protein kinase 3 phosphorylated at tyrosine 204;
      Serine/threonine-protein kinase PAK 1 phosphorylated at serine 219;
      Serine/threonine-protein kinase PAK 1 phosphorylated at threonine 229;
      Serine/threonine-protein kinase PAK 1 phosphorylated at threonine 230;
      Eukaryotic translation initiation factor 4E-binding protein 1 phosphorylated at threonine 77; and/or
      Dual specificity mitogen-activated protein kinase kinase 1 phosphorylated at serine 377; and/or
   (ii) expression of one or more of the following proteins:
      Lysine-specific histone demethylase;
      Serine/threonine-protein kinase mTOR;
      Dual specificity mitogen-activated protein kinase kinase 2;
      Ribosomal protein S6 kinase beta-1;
      Mitogen-activated protein kinase 1;
      Dual specificity mitogen-activated protein kinase kinase 4;
      Ribosomal protein S6 kinase alpha-1;
      Dual specificity mitogen-activated protein kinase kinase 3;
      Ribosomal protein S6 kinase alpha-5;
      Dual specificity mitogen-activated protein kinase kinase 5;
      Phosphatidylinositol 4,5-bisphosphate 3-kinase catalytic subunit delta isoform; and/or
      Dual specificity mitogen-activated protein kinase kinase 1; and/or
(iii) mutation of, or chromosomal rearrangement involving, one or more of the following genes: KMT2A, NPM1, KRAS, NRAS, DNMT3A, IDH2, TP53, TET2, FLT3 and/or STAG2; and/or
(iv) classified as M5 by the French-American-British (FAB) AML classification scheme;
   wherein detecting the presence and/or level of one or more of said markers selects the subject for treatment with the epigenetic agent followed by treatment with the kinase inhibitor.

The selection of the subject for treatment with the epigenetic agent followed by treatment with the kinase inhibitor may be performed based on associations of the markers with sequential treatment sensitivity and/or resistance as described elsewhere herein.

In an alternative configuration, the sixth aspect may be a computer implemented method of selecting a subject with cancer for treatment with an epigenetic agent which inhibits a chromatin-modifying enzyme and/or which promotes cellular differentiation, followed by treatment with a kinase inhibitor, wherein the method comprises:
detecting, in data obtained from a sample of cancer cells obtained from the subject, the presence and/or level of one or more of the following markers:
   (i) phosphorylation at one or more of the following phosphorylation sites:
      Mitogen-activated protein kinase kinase kinase 2 phosphorylated at threonine 339;
      Ribosomal protein S6 kinase alpha-3 phosphorylated at threonine 391;
      Receptor tyrosine-protein kinase erbB-2 phosphorylated at serine 1073;
      Mitogen-activated protein kinase kinase kinase 3 phosphorylated at serine 129;
      Epidermal growth factor receptor phosphorylated at serine 991;
      PH domain leucine-rich repeat-containing protein phosphatase 1 phosphorylated at serine 1524;
      Dual specificity mitogen-activated protein kinase kinase 1 phosphorylated at serine 222;
      Dual specificity mitogen-activated protein kinase kinase 1 phosphorylated at serine 226;
      Kinase suppressor of Ras 1 phosphorylated at serine 202;
      Dual specificity mitogen-activated protein kinase kinase 1 phosphorylated at serine 230;
      Serine/threonine-protein kinase B-raf phosphorylated at threonine 753;
      Mitogen-activated protein kinase kinase kinase 4 phosphorylated at serine 499;
      Serine/threonine-protein kinase mTOR phosphorylated at threonine 2446;
      Serine/threonine-protein kinase mTOR phosphorylated at serine 2448;
      Serine/threonine-protein kinase A-Raf phosphorylated at serine 580;
      RAF proto-oncogene serine/threonine-protein kinase phosphorylated at serine 619;
      PH domain leucine-rich repeat-containing protein phosphatase 2 phosphorylated at serine 1210;
      Mitogen-activated protein kinase kinase kinase 20 at serine 648;
      Mitogen-activated protein kinase kinase kinase 20 at serine 649;
      Dual specificity mitogen-activated protein kinase kinase 2 phosphorylated at threonine 13;
      Glycogen synthase kinase-3 beta phosphorylated at serine 215;
      Glycogen synthase kinase-3 alpha phosphorylated at serine 278;
      Mitogen-activated protein kinase 3 phosphorylated at threonine 207;
      Serine/threonine-protein kinase N1 phosphorylated at serine 584;
      Tyrosine-protein phosphatase non-receptor type 7 phosphorylated at serine 359;
      RAC-alpha serine/threonine-protein kinase phosphorylated at serine 457;
      RAC-beta serine/threonine-protein kinase phosphorylated at serine 458;
      RAC-beta serine/threonine-protein kinase phosphorylated at tyrosine 438;
      Mitogen-activated protein kinase 3 phosphorylated at threonine 202;
      Mitogen-activated protein kinase 3 phosphorylated at tyrosine 204;
      Serine/threonine-protein kinase PAK 1 phosphorylated at serine 219;
      Serine/threonine-protein kinase PAK 1 phosphorylated at threonine 229;
      Serine/threonine-protein kinase PAK 1 phosphorylated at threonine 230;
      Eukaryotic translation initiation factor 4E-binding protein 1 phosphorylated at threonine 77; and/or
      Dual specificity mitogen-activated protein kinase kinase 1 phosphorylated at serine 377; and/or
   (ii) expression of one or more of the following proteins:
      Lysine-specific histone demethylase;
      Serine/threonine-protein kinase mTOR;
      Dual specificity mitogen-activated protein kinase kinase 2;
      Ribosomal protein S6 kinase beta-1;
      Mitogen-activated protein kinase 1;
      Dual specificity mitogen-activated protein kinase kinase 4;
      Ribosomal protein S6 kinase alpha-1;
      Dual specificity mitogen-activated protein kinase kinase 3;
      Ribosomal protein S6 kinase alpha-5;
      Dual specificity mitogen-activated protein kinase kinase 5;
      Phosphatidylinositol 4,5-bisphosphate 3-kinase catalytic subunit delta isoform; and/or
      Dual specificity mitogen-activated protein kinase kinase 1; and/or
(iii) mutation of, or chromosomal rearrangement involving, one or more of the following genes: KMT2A, NPM1, KRAS, NRAS, DNMT3A, IDH2, TP53, TET2, FLT3 and/or STAG2; and/or
(iv) classified as M5 by the French-American-British (FAB) AML classification scheme;
   wherein detecting the presence and/or level of one or more of said markers selects the subject for treatment with the epigenetic agent followed by treatment with the kinase inhibitor.

The method may comprise
(a) receiving in a computer data identifying a patient who is suffering from cancer and data representing, in a sample of cancer cells obtained from the subject, the presence and/or level of one or more of the following markers:
   (i) phosphorylation at one or more of the following phosphorylation sites:
      Mitogen-activated protein kinase kinase kinase 2 phosphorylated at threonine 339;
      Ribosomal protein S6 kinase alpha-3 phosphorylated at threonine 391;
      Receptor tyrosine-protein kinase erbB-2 phosphorylated at serine 1073;
      Mitogen-activated protein kinase kinase kinase 3 phosphorylated at serine 129;
      Epidermal growth factor receptor phosphorylated at serine 991;
      PH domain leucine-rich repeat-containing protein phosphatase 1 phosphorylated at serine 1524;
      Dual specificity mitogen-activated protein kinase kinase 1 phosphorylated at serine 222;
      Dual specificity mitogen-activated protein kinase kinase 1 phosphorylated at serine 226;
      Kinase suppressor of Ras 1 phosphorylated at serine 202;
      Dual specificity mitogen-activated protein kinase kinase 1 phosphorylated at serine 230;
      Serine/threonine-protein kinase B-raf phosphorylated at threonine 753;
      Mitogen-activated protein kinase kinase kinase 4 phosphorylated at serine 499;
      Serine/threonine-protein kinase mTOR phosphorylated at threonine 2446;
      Serine/threonine-protein kinase mTOR phosphorylated at serine 2448;
      Serine/threonine-protein kinase A-Raf phosphorylated at serine 580;
      RAF proto-oncogene serine/threonine-protein kinase phosphorylated at serine 619;
      PH domain leucine-rich repeat-containing protein phosphatase 2 phosphorylated at serine 1210;
      Mitogen-activated protein kinase kinase kinase 20 at serine 648;
      Mitogen-activated protein kinase kinase kinase 20 at serine 649;
      Dual specificity mitogen-activated protein kinase kinase 2 phosphorylated at threonine 13;
      Glycogen synthase kinase-3 beta phosphorylated at serine 215;
      Glycogen synthase kinase-3 alpha phosphorylated at serine 278;
      Mitogen-activated protein kinase 3 phosphorylated at threonine 207;
      Serine/threonine-protein kinase N1 phosphorylated at serine 584;
      Tyrosine-protein phosphatase non-receptor type 7 phosphorylated at serine 359;
      RAC-alpha serine/threonine-protein kinase phosphorylated at serine 457;
      RAC-beta serine/threonine-protein kinase phosphorylated at serine 458;
      RAC-beta serine/threonine-protein kinase phosphorylated at tyrosine 438;
      Mitogen-activated protein kinase 3 phosphorylated at threonine 202;
      Mitogen-activated protein kinase 3 phosphorylated at tyrosine 204;
      Serine/threonine-protein kinase PAK 1 phosphorylated at serine 219;
      Serine/threonine-protein kinase PAK 1 phosphorylated at threonine 229;
      Serine/threonine-protein kinase PAK 1 phosphorylated at threonine 230;
      Eukaryotic translation initiation factor 4E-binding protein 1 phosphorylated at threonine 77; and/or
      Dual specificity mitogen-activated protein kinase kinase 1 phosphorylated at serine 377; and/or
   (ii) expression of one or more of the following proteins:
      Lysine-specific histone demethylase;
      Serine/threonine-protein kinase mTOR;
      Dual specificity mitogen-activated protein kinase kinase 2;
      Ribosomal protein S6 kinase beta-1;
      Mitogen-activated protein kinase 1;
      Dual specificity mitogen-activated protein kinase kinase 4;
      Ribosomal protein S6 kinase alpha-1;
      Dual specificity mitogen-activated protein kinase kinase 3;
      Ribosomal protein S6 kinase alpha-5;
      Dual specificity mitogen-activated protein kinase kinase 5;
      Phosphatidylinositol 4,5-bisphosphate 3-kinase catalytic subunit delta isoform; and/or
      Dual specificity mitogen-activated protein kinase kinase 1; and/or
   (iii) mutation of, or chromosomal rearrangement involving, one or more of the following genes: KMT2A, NPM1, KRAS, NRAS, DNMT3A, IDH2, TP53, TET2, FLT3 and/or STAG2; and/or
   (iv) classified as M5 by the French-American-British (FAB) AML classification scheme; and
(b) based on the data representing the presence and/or level of one or more of the markers, generating output data associated with the individual patient to indicate that the patent may be effectively treated by treatment with the epigenetic agent followed by treatment with the kinase inhibitor.

Step (b) may be performed based on associations of the markers with sequential treatment sensitivity and/or resistance as described elsewhere herein.

The methods of the invention may therefore be implemented on a computer, using a computer program product. The computer program product may include computer code arranged to instruct a computer to perform the functions of one or more of the various methods described above. The computer program and/or the code for performing such methods may be provided to an apparatus, such as a computer, on a computer readable medium or computer program product. The computer readable medium may be transitory or non-transitory. The computer readable medium could be, for example, an electronic, magnetic, optical, electromagnetic, infrared, or semiconductor system, or a propagation medium for data transmission, for example for downloading the code over the Internet. Alternatively, the computer readable medium could take the form of a physical computer readable medium such as semiconductor or solid-state memory, magnetic tape, a removable computer diskette, a random access memory (RAM), a read-only memory (ROM), a rigid magnetic disc, and an optical disk, such as a CD-ROM, CD-R/W or DVD.

An apparatus such as a computer may be configured in accordance with such code to perform one or more processes in accordance with the various methods discussed herein. In one arrangement the apparatus comprises a processor, memory, and a display. Typically, these are connected to a central bus structure, the display being connected via a display adapter. The system can also comprise one or more input devices (such as a mouse and/or keyboard) and/or a communications adapter for connecting the apparatus to other apparatus or networks. Such an apparatus may take the form of a data processing system. Such a data processing system may be a distributed system. For example, such a data processing system may be distributed across a network.

The present invention also provides software for performing the computer-implemented methods disclosed herein.

The method of selecting a subject may alternatively be described as a method for identifying a patient suffering from cancer suitable for pre-treatment with an epigenetic agent prior to treatment with a protein kinase inhibitor. The invention therefore also provides a method for identifying a patient suffering from cancer suitable for pre-treatment with an epigenetic inhibitor which inhibits a chromatin-modifying enzyme and/or which promotes cellular differentiation, prior to treatment with a protein kinase inhibitor, comprising the steps of assaying a sample of cells from the patient for the presence of one or more markers selected from the group consisting of:
(i) phosphorylation at one or more of the following phosphorylation sites:
   Mitogen-activated protein kinase kinase kinase 2 phosphorylated at threonine 339;
   Ribosomal protein S6 kinase alpha-3 phosphorylated at threonine 391;
   Receptor tyrosine-protein kinase erbB-2 phosphorylated at serine 1073;
   Mitogen-activated protein kinase kinase kinase 3 phosphorylated at serine 129;
   Epidermal growth factor receptor phosphorylated at serine 991;
   PH domain leucine-rich repeat-containing protein phosphatase 1 phosphorylated at serine 1524;
   Dual specificity mitogen-activated protein kinase kinase 1 phosphorylated at serine 222;
   Dual specificity mitogen-activated protein kinase kinase 1 phosphorylated at serine 226;
   Kinase suppressor of Ras 1 phosphorylated at serine 202;
   Dual specificity mitogen-activated protein kinase kinase 1 phosphorylated at serine 230;
   Serine/threonine-protein kinase B-raf phosphorylated at threonine 753;
   Mitogen-activated protein kinase kinase kinase 4 phosphorylated at serine 499;
   Serine/threonine-protein kinase mTOR phosphorylated at threonine 2446;
   Serine/threonine-protein kinase mTOR phosphorylated at serine 2448;
   Serine/threonine-protein kinase A-Raf phosphorylated at serine 580;
   RAF proto-oncogene serine/threonine-protein kinase phosphorylated at
   serine 619;
   PH domain leucine-rich repeat-containing protein phosphatase 2 phosphorylated at serine 1210;
   Mitogen-activated protein kinase kinase kinase 20 at serine 648;
   Mitogen-activated protein kinase kinase kinase 20 at serine 649;
   Dual specificity mitogen-activated protein kinase kinase 2 phosphorylated at threonine 13;
   Glycogen synthase kinase-3 beta phosphorylated at serine 215;
   Glycogen synthase kinase-3 alpha phosphorylated at serine 278;
   Mitogen-activated protein kinase 3 phosphorylated at threonine 207;
   Serine/threonine-protein kinase N1 phosphorylated at serine 584;
   Tyrosine-protein phosphatase non-receptor type 7 phosphorylated at serine 359;
   RAC-alpha serine/threonine-protein kinase phosphorylated at serine 457;
   RAC-beta serine/threonine-protein kinase phosphorylated at serine 458;
   RAC-beta serine/threonine-protein kinase phosphorylated at tyrosine 438;
   Mitogen-activated protein kinase 3 phosphorylated at threonine 202;
   Mitogen-activated protein kinase 3 phosphorylated at tyrosine 204;
   Serine/threonine-protein kinase PAK 1 phosphorylated at serine 219;
   Serine/threonine-protein kinase PAK 1 phosphorylated at threonine 229;
   Serine/threonine-protein kinase PAK 1 phosphorylated at threonine 230;
   Eukaryotic translation initiation factor 4E-binding protein 1 phosphorylated at threonine 77; and/or
   Dual specificity mitogen-activated protein kinase kinase 1 phosphorylated at serine 377; and/or
(ii) expression of one or more of the following proteins:
   Lysine-specific histone demethylase;
   Serine/threonine-protein kinase mTOR;
   Dual specificity mitogen-activated protein kinase kinase 2;
   Ribosomal protein S6 kinase beta-1;
   Mitogen-activated protein kinase 1;
   Dual specificity mitogen-activated protein kinase kinase 4;
   Ribosomal protein S6 kinase alpha-1;
   Dual specificity mitogen-activated protein kinase kinase 3;
   Ribosomal protein S6 kinase alpha-5;
   Dual specificity mitogen-activated protein kinase kinase 5;
   Phosphatidylinositol 4,5-bisphosphate 3-kinase catalytic subunit delta isoform; and/or
   Dual specificity mitogen-activated protein kinase kinase 1; and/or
(iii) mutation of, or chromosomal rearrangement involving, one or more of the following genes: KMT2A, NPM1, KRAS, NRAS, DNMT3A, IDH2, TP53, TET2, FLT3 and/or STAG2; and/or
(iv) classified as M5 by the French-American-British (FAB) AML classification scheme;

The cancer may be selected from acute myeloid leukaemia, oesophageal cancer, breast cancer and liver cancer. The cancer may be acute myeloid leukaemia. The cancer may be classified as being a FAB M4, M5 or M6 subtype acute myeloid leukaemia. Cancer classified as being a FAB M4, M5 or M6 subtype acute myeloid leukaemia may be associated with cells sensitive to sequential treatment. Cancer classified as being a FAB M5 subtype acute myeloid leukaemia may be associated with cells sensitive to sequential treatment. The cancer may be classified as being KMT2A rearranged (KMT2Ar) acute lymphoblastic leukaemia (ALL). Cancer classified as being KMT2Ar ALL may be associated with cells sensitive to sequential treatment.

Acute myeloid leukaemia (AML), also known as acute myelogenous leukaemia, acute myeloblastic leukaemia, acute granulocytic leukemia or acute nonlymphocytic leukemia, is an aggressive cancer of the blood and bone marrow. AML is characterised by excessive production of immature white blood cells, known as myeloblasts, by bone marrow. In healthy individuals, blasts normally develop into mature white blood cells. In AML, however, the blasts do not differentiate normally but remain at a premature arrested state of development.

In AML, the bone marrow may also make abnormal red blood cells and platelets. The number of these abnormal cells increases rapidly, and the abnormal cells begin to crowd out the normal white blood cells, red blood cells and platelets that the body needs. If left untreated, acute myeloid leukaemia is rapidly fatal.

Various classification systems have been devised for classifying AML into disease subtypes, with the aim of enabling more accurate prognosis of disease progression and identification of the optimal form of treatment. The earliest system was the French-American-British (FAB) classification, first devised in the 1970s by a group of French, American and British leukaemia experts. This system divides AML into subtypes according to the type of cell from which the leukaemia has developed, and the stage of maturity reached by the myeloblast cells at the point of arrest. Subtypes M0 to M5 originate from precursors of white blood cells and range from undifferentiated myeloblastic leukaemia (M0) to monocytic leukaemia (M5). Subtype M6 originates in very early forms of red blood cells (erythroid leukaemia), whilst subtype M7 AML starts in early forms of cells that form platelets (megakaryoblastic leukaemia).

Under the FAB system, AML is categorised by visual inspection of cytomorphological features under the microscope, and by identification of various chromosomal abnormalities. An updated version of the FAB categorisation was published in 1985—see Bennett et al, Proposed revised criteria for the classification of acute myeloid leukaemia, Ann Intern Med 1985; 103(4): 620-625.

Since the FAB system was first devised in the 1970s, the level of knowledge in the field has moved on considerably. Whilst the system has been updated to incorporate some of this knowledge, it was felt to be necessary to create a new classification system, taking into account additional factors now known to affect prognosis and to be determinative in optimising effective treatment.

The World Health Organization (WHO) classification system accordingly divides AML into several broad groups. These include:—
AML with recurrent genetic abnormalities, meaning with specific chromosomal changes
AML with multilineage dysplasia
AML, related to previous therapy that is damaging to cells, including chemotherapy and radiotherapy, also called therapy-related myeloid neoplasm
AML that is not otherwise categorized—including:—
   Undifferentiated AML (M0)
   AML with minimal maturation (M1)
   AML with maturation (M2)
   Acute myelomonocytic leukemia (M4)
   Acute monocytic leukemia (M5)
   Acute erythroid leukemia (M6)
   Acute megakaryoblastic leukemia (M7)
   Acute basophilic leukemia
   Acute panmyelosis with fibrosis
   Myeloid sarcoma (also known as granulocytic sarcoma or chloroma)

In addition to these two main classification systems, AML is further categorised and subtyped by reference to specific molecular markers which are found to correlate with certain phenotypes and outcomes. For example, patients with mutations in the NPM1 gene or CEBPA genes are known to have a better long term outcome, whilst patients with certain mutations in FLT3 have been found to have a worse prognosis—see Yohe et al, J Clin Med. 2015 Mar. 4(3): 460-478.

Histone-lysine N-methyltransferase 2A also known as acute lymphoblastic leukaemia 1 (ALL-1), myeloid/lymphoid or mixed-lineage leukaemia 1 (MLL1), or zinc finger protein HRX (HRX) is an enzyme that in humans is encoded by the KMT2A gene. Rearrangements of the KMT2A gene are associated with aggressive acute leukaemia, both lymphoblastic and myeloid and can therefore cause KMT2A rearranged (KMT2Ar) acute lymphoblastic leukaemia (ALL).

Most proteins are modified in some way by the addition of functional groups and such modifications are effected by protein modifying enzymes. Protein modifications that can be detected by mass spectrometry include phosphorylation, glycosylation, acetylation, methylation and lipidation. These protein modifications have various biological roles in the cell. The modification sites may therefore be sites of post-translational modifications. For example, the modification sites may be sites may be sites of phosphorylation, glycosylation, acetylation, methylation and lipidation. The modification sites are typically protein and/or peptide modification sites. A modification site may be one or more amino acid residues of a peptide or protein to which a functional group such as a phosphate group is added to the peptide or protein. Alternative functional groups include carbohydrates, acetyl groups, methyl groups and lipids. By “protein modifying enzyme” is therefore meant an enzyme which catalyses a reaction involving the addition of a functional group to a protein or peptide. A “modified peptide” is defined herein as a peptide which has been modified by the addition or removal of a functional group. A “protein modifying enzyme” is defined herein as an enzyme which catalyses a reaction involving the addition or removal of a functional group to a protein or peptide.

A “peptide” as defined herein is a short amino acid sequence and includes oligopeptides and polypeptides. Typically, such peptides are between about 5 and 30 amino acids long, for example from 6 or 7 to 25, 26 or 27 amino acids, from 8, 9 or 10 to 20 amino acids, from 11 or 12 to 18 amino acids or from 14 to 16 amino acids, for example 15 amino acids. However, shorter and longer peptides, such as between about 2 and about 50, for example from about 3 to about 35 or 40 or from about 4 to about 45 amino acids can also be used. Typically, the peptide is suitable for mass spectrometric analysis, that is the length of the peptide is such that the peptide is suitable for mass spectrometric analysis. The length of the peptide that can be analysed is limited by the ability of the mass spectrometer to sequence such long peptides. In certain cases polypeptides of up to 300 amino acids can be analysed, for example from 50 to 250 amino acids, from 100 to 200 amino acids or from 150 to 175 amino acids.

As described herein, the methods may be based on the analysis of peptides and/or modified peptides which are identified and/or quantified using MS-based techniques. In some embodiments, the method of the invention therefore includes a step of identifying and/or quantifying peptides and/or modified peptides in a sample using mass spectrometry (MS).

The method may be based on the analysis of phosphorylated peptides. Phosphorylated peptides contain one or more amino acid which is phosphorylated (i.e. a phosphate (PO₄) group has been added to that amino acid). Such phosphorylated amino acids are referred to herein as “phosphorylation sites”. When a peptide is phosphorylated by a particular protein kinase, it is referred to as a “substrate” of that protein kinase. In relation to this embodiment of the invention, the term “phosphoprotein” is used herein to refer to a phosphorylated protein and the term “phosphopeptide” is used herein to refer to a phosphorylated peptide.

The sample used in the methods of the invention can be any suitable sample which contains cells and/or peptides from a patient. The sample may be termed a “biological” sample. The patient may be a human or animal suffering from or suspected of suffering from cancer, such as acute myeloid leukaemia. Where the method involves control samples these may or may not be from a human or animal suffering from or suspected of suffering from cancer, such as acute myeloid leukaemia (the control sample may be from a healthy individual). The invention thus encompasses the use of samples obtained from human and non-human sources.

The present invention finds use in the field of personalized medicine. Typically, therefore, the biological sample is derived from a human, and can be, for example, a sample of a bodily fluid such as bone marrow or blood, or another tissue. Typically, the biological sample is from a tissue, typically a primary tissue, or from a tissue which has undergone processing after isolation such as culturing of cells, such as leukemia cells, or storage. For example, the sample can be a tissue from a human or animal. The human or animal can be healthy or diseased. In an embodiment, the human has been diagnosed with or is suspected as having acute myeloid leukemia (AML). Typically, the sample comprises leukemia cells. The leukemia cells may be myeloblasts, abnormal red blood cells or platelets. Accordingly, the tissue may be from a peripheral blood sample or from a bone marrow sample. The sample may be a peripheral blood sample or a bone marrow sample. The sample may be leukaemia cells which have previously been obtained from the patient. This invention is applicable to all AML patients, including newly-diagnosed (untreated) AML patients, AML patients who have undergone or are undergoing other forms of treatment, and relapsed AML patients. The AML patient may be newly diagnosed. The patient may be newly diagnosed with AML that is FAB M4, M5 or M6 subtype acute myeloid leukaemia. The patient may be newly diagnosed with AML based on analysis of the sample used in the method of the invention. The patient may be newly diagnosed with AML based on analysis of an aliquot or portion of the sample used in the method of the invention. The patient may be newly diagnosed with AML based on analysis of a second sample obtained at the same or at a similar time as the sample used in the method of the invention. The sample may have been obtained prior to diagnosis of AML and/or prior to treatment for AML. The sample may have been obtained after diagnosis of AML and/or after treatment for AML.

The method may comprise performing an in vitro assay to detect the presence and/or level of one or more of the markers described herein. The method may comprise performing an in vitro assay to detect the level one or more proteins and/or the level of phosphorylation at the one or more phosphorylation sites in the sample obtained from the patient. Said assay may be an LC-MS/MS assay or an assay based on affinity reagents such as aptamers, molecularly imprinted polymers, or antibodies (immunochemical assays). The assay based on affinity reagents may be a Western blot assay, an ELISA assay or a reversed phase protein assay. The assay can be carried out by any method involving mass spectrometry (MS), such as liquid chromatography-mass spectrometry (LC-MS). The LC-MS or LC-MS/MS is typically label-free MS but techniques that use isotope labelling as the basis for to detecting the level one or more proteins and/or the level of phosphorylation at the one or more phosphorylation sites can also be used as the basis for the analysis. The assay may be an LC-MS/MS assay. The assay may comprise using a label-free mass spectrometry approach as previously described in Casado et al., 2018 Leukemia 32, 1818-1822 and/or WO 2018/234404, both of which are incorporated by reference herein in their entirety.

Peptides can be obtained from the sample using any suitable method known in the art. In one embodiment, prior to step (a), the method of the invention comprises:
(1) lysing cells in the sample;
(2) extracting the proteins from the lysed cells obtained in step (1); and
(3) cleaving said proteins into peptides.

In step (1) of this embodiment of the invention, the cells in the sample are lysed, or split open. The cells can be lysed using any suitable means known in the art, for example using physical methods such as mechanical lysis (for example using a Waring blender), liquid homogenization, sonication or manual lysis (for example using a pestle and mortar) or detergent-based methods such as CHAPS or Triton-X. Typically, the cells are lysed using a denaturing buffer such as a urea-based buffer.

In step (2) of this embodiment of the invention, proteins are extracted from the lysed cells obtained in step (1). In other words, the proteins are separated from the other components of the lysed cells.

In step (3) of this embodiment of the invention, the proteins from the lysed cells are cleaved into peptides. In other words, the proteins are broken down into shorter peptides. Protein breakdown is also commonly referred to as digestion. Protein cleavage can be carried out in the present invention using any suitable agent known in the art.

Protein cleavage or digestion is typically carried out using a protease. Any suitable protease can be used in the present invention. In the present invention, the protease is typically trypsin, chymotrypsin, Arg-C, pepsin, V8, Lys-C, Asp-C and/or AspN. Alternatively, the proteins can be cleaved chemically, for example using hydroxylamine, formic acid, cyanogen bromide, BNPS-skatole, 2-nitro-5-thiocyanobenzoic acid (NTCB) or any other suitable agent.

Peptides (including phosphorylated peptides) detected by carrying out liquid chromatography-tandem mass spectrometry (LC-MS/MS) may be compared to a known reference database in order to identify the peptides (including phosphorylated peptides). This step is typically carried out using a commercially available search engine, such as, but not restricted to, the MASCOT, ProteinProspector, Andromeda, or Sequest search engines. Other computer programmes and workflows, such as MaxQuant [Nature Biotechnology 26, 1367-1372 (2008)] may be used to quantify peptides.

The computer program named PESCAL (Cutillas and Vanhaesebroeck, Molecular & Cellular Proteomics 6, 1560-1573 (2007)) automates the quantification of each peptide (including phosphorylated peptides) listed in the database in LC-MS runs of modified peptides (including phosphorylated peptides) taken from biological experiments. For these biological experiments, proteins in cell lysates are digested using trypsin or other suitable proteases. Peptide (such as phosphopeptide) internal standards, which are reference modified peptides (including reference phosphorylated peptides), are spiked at known amounts in all the samples to be compared. Peptides (including phosphorylated peptides) in the resultant peptide mixture may be enriched using a simple-to-perform IMAC or TiO₂ extraction step. Enriched peptides (including phosphorylated peptides) are analysed in a single LC-MS run of typically but not restricted to about 120 minutes (total cycle). PESCAL then constructs extracted ion chromatograms (XIC, i.e, an elution profile) for each of the peptides (including phosphorylated peptides) present in the database across all the samples that are to be compared. The program also calculates the peak height and area under the curve of each XIC. The data is normalised by dividing the intensity reading (peak areas or heights) of each peptide (including phosphopeptide) analyte by those of the peptide (including phosphopeptide) ISs.

Quantification of modifications such as phosphorylation can also be carried out using MS techniques that use isotope labels for quantification, such as metabolic labeling (e.g., stable isotope labeled amino acids in culture, (SILAC); Olsen, J. V. et al. Cell 127, 635-648 (2006)), and chemical derivatization (e.g., iTRAQ (Ross, P. L.; et al. Mol Cell Proteomics 2004, 3, (12), 1154-69), ICAT (Gygi, S. P. et al. Nat Biotechnol 17, 994-999 (1999)), TMT (Dayon L et al, Anal Chem. 2008 Apr. 15; 80(8):2921-31) techniques. Protein modifications can be quantified with LC-MS techniques that measure the intensities of the unfragmented ions or with LC-MS/MS techniques that measure the intensities of fragment ions (such as Selected Reaction Monitoring (SRM), also named multiple reaction monitoring (MRM) and parallel-reaction monitoring (PRM)).

Other computer programs such as Skyline are alternatives to PESCAL.

The method may therefore comprise normalising the level of each biomarker to the level of an internal standard, such as an isotopically labelled standard. This may provide absolute quantification of the level of the biomarker.

LC-MS/MS may be suitable for use in situations where there is access to the equipment required in, for example, in hospital or in centralised laboratories. However, more conveniently, the levels of the at least one biomarker in the samples may be measured using assays based on affinity reagents such as immunoassays. Immunoassays have the potential to be miniaturised to run on a microfluidics device or test-strip and may be more suited for clinical point-of-care applications. Embodiments of the invention which incorporate an immunoassay may therefore be used in situ by a primary healthcare provider for assistance in prescribing a statin for an individual patient.

The levels of the at least one biomarker may be measured using a homogeneous or heterogeneous immunoassay.

Thus, in some embodiments, the levels of the or each biomarker may be measured in solution by binding to labelled antibodies, aptamers or molecular imprinted polymers that are present in excess, whereby binding alters detectable properties of the label. The amount of a specific biomarker present will therefore affect the amount of the label with a particular detectable property. As is well known in the art, the label may comprise a radioactive label, a fluorescent label or an enzyme having a chromogenic or chemiluminescent substrate that is coloured or caused or allowed to fluoresce when acted on by the enzyme.

The antibodies may be polyclonal or monoclonal with specificity for the biomarker. In some embodiments, monoclonal antibodies may be used.

Alternatively, a heterogeneous format may be used in which the at least one biomarker is captured by surface-bound antibodies for separation and quantification. In some embodiments, a sandwich assay may be used in which a surface-bound biomarker is quantified by binding a labelled secondary antibody.

Suitably, the immunoassay may comprise an enzyme immunoassay (EIA) in which the label is an enzyme such, for example, as horseradish peroxidase (HRP). Suitable substrates for HRP are well known in the art and include, for example, ABTS, OPD, AmplexRed, DAB, AEC, TMB, homovanillic acid and luminol. In some embodiments, an ELISA immunoassay may be used; a sandwich ELISA assay may be particularly preferred.

The immunoassay may be competitive or non-competitive. Thus, in some embodiments, the amounts of the at least one biomarker may be measured directly by a homogeneous or heterogeneous method, as described above. Alternatively, the biomarker in the samples may be sequestered in solution with a known amount of antibody which is present in excess, and the amount of antibody remaining then determined by binding to surface-bound biomarker to give an indirect read-out of the amount of biomarker in the original sample. In another variant, the at least one biomarker may be caused to compete for binding to a surface bound antibody with a known amount of a labelled biomarker.

The surface bound antibodies or biomarker may be immobilised on any suitable surface of the kind known in the art. For instance, the antibodies or biomarker may be immobilised on a surface of a well or plate or on the surface of a plurality of magnetic or non-magnetic beads.

In some embodiments, the immunoassay may be a competitive assay, further comprising a known amount of the biomarker, which is the same as the one to be quantitated in the sample, but tagged with a detectable label. The labelled biomarker may be affinity-bound to a suitable surface by an antibody to the biomarker. Upon adding the sample a proportion of the labelled biomarker may be displaced from the surface-bound antibodies, thereby providing a measure of the level of biomarker in the sample.

In some embodiments, the immunoassay may comprise surface-bound biomarker, which is the same as the biomarker that is to be quantitated in the sample, and a known amount of antibodies to the biomarker in solution in excess. The sample is first mixed with the antibodies in solution such that a proportion of the antibodies bind with the biomarker in the sample. The amount of unbound antibodies remaining can then be measured by binding to the surface-bound biomarker.

In some embodiments, the immunoassay may comprise a labelled secondary antibody to the biomarker or to a primary antibody to the biomarker for quantifying the amount of the biomarker bound to surface-bound antibodies or the amount of primary antibody bound to the biomarker immobilised on a surface.

Measuring biomarker levels may be by equipment for measuring the level of a specific biomarker in a sample comprising a sample collection device and an immunoassay. The equipment may further comprise a detector for detecting labelled biomarker or labelled antibodies to the biomarker in the immunoassay. Suitable labels are mentioned above, but in a preferred embodiment, the label may be an enzyme having a chromogenic or chemiluminescent substrate that is coloured or caused or allowed to fluoresce when acted on by the enzyme.

The immunoassay or equipment may be incorporated into a miniaturised device for measuring the level of at least one biomarker in a biological sample. Suitably, the device may comprise a lab-on-a-chip.

Measuring biomarker levels may be by a device for measuring the level of at least one biomarker in a sample obtained from a patient, the device comprising one or more parts defining an internal channel having an inlet port and a reaction zone, in which a biomarker in a sample may be reacted with an immobilised primary antibody for the biomarker for capturing the biomarker, or a primary antibody for the biomarker in excess in solution after mixing with the sample upstream of the reaction zone may be reacted with biomarker, which is the same as the one to be measured in the sample, but immobilised on a surface within the reaction zone, for quantifying directly or indirectly the amount of the biomarker in the sample.

The captured biomarker or primary antibody may then be detected using a secondary antibody to the biomarker or primary antibody, which is tagged with an enzyme.

As described above, the enzyme may have a chromogenic or chemiluminescent substrate that is coloured or caused or allowed to fluoresce when acted on by the enzyme. Suitably, the one or more parts of the device defining the channel, at least adjacent the reaction zone, may be transparent to light, at least in a range of wavelengths encompassing the colour or fluorescence of the substrate to allow detection of a reaction between the biomarker or primary antibody and the secondary antibody using a suitable detector such, for example, as a photodiode, positioned outside the channel or further channel.

In some embodiments, the device may comprise a plurality of channels, each with its own inlet port, for measuring the levels of a plurality of different biomarkers in the sample in parallel. Therefore, each channel may include a different respective immobilised primary antibody or biomarker. Suitably, the device may comprise one or more selectively operable valves associated with the one or more inlet ports for controlling the admission of a sequence of different reagents into to the channels such, for example, as the sample, wash solutions, primary antibody, secondary antibody and enzyme substrate.

The device therefore may comprise a microfluidics device. The channel may include a reaction zone. Microfluidics devices are known to those skilled in the art. A review of microfluidic immunoassays or protein diagnostic chip microarrays is provided by Chin et al. 2012. Lab on a Chip. 2012; 12:2118-2134. A microfluidics device suitable for carrying out an ELISA immunoassay at a point-of-care is disclosed by Chan C D, Laksanasopin T, Cheung Y K, Steinmiller D et al. “Microfluidics-based diagnostics of infectious diseases in the developing world”. Nature Medicine. 2011; 17(8):1015-1019, the contents of which are incorporated herein by reference.

The epigenetic agent may inhibit a chromatin-modifying enzyme. The epigenetic agent may be described as an inhibitor of a chromatin modifying enzyme. The chromatin modifying enzyme may contribute to cellular differentiation. Typically, the chromatin modifying enzyme may inhibit cell differentiation, such that an inhibitor of the chromatin modifying enzyme promotes cell differentiation. The chromatin modifying enzyme may maintain a pluripotent phenotype, for example such as LSD1 in hematopoietic cells, and/or may be overexpressed in undifferentiated cells. The chromatin modifying enzyme may be a histone deacetylase, histone demethylase, lysine demethylase or a component of a polycomb repressive complex. The chromatin-modifying enzyme may be selected from the group consisting of lysine-specific histone demethylase 1A (LSD1, also known as KDM1A), DNA methyltransferase (DNMTA), histone deacetylase (HDAC), enhancer of zeste homolog 2 (EZH2) or disruptor of telomeric silencing 1-like protein (DOT1L). Preferably the chromatin-modifying enzyme is LSD1.

The epigenetic agent may promote cellular differentiation. The epigenetic agent may be described as a promoter of cellular differentiation. Alternatively, the epigenetic agent may not be a promoter of cellular differentiation. The cellular differentiation may be differentiation of the cell type affected by the cancer. Where the cancer is AML, the cellular differentiation may be myeloid cell differentiation. Where the cancer is oesophageal cancer, the cellular differentiation may be oesophageal cell differentiation. Where the cancer is oesophageal cancer, the cellular differentiation may be breast cell differentiation. Where the cancer is liver cancer, the cellular differentiation may be liver cell differentiation. The epigenetic agent may reduce the activity of kinase pro-survival pathways such as PI3K-AKT. The epigenetic agent may increase expression of differentiation markers. The differentiation markers may be CD11b, CD64, CD14, CD117, CD16 and/or CD15. The differentiation markers may be CD11b and/or CD64. The epigenetic agent may be selected from the group consisting of GSK2879552, ORY1001 (Iadademstat), GSK-LSD1, CC90011, IMG-7289 (Bomedemstat), all-trans retinoic acid (ATRA), decitabine, vorinostat, CPI-1205 and pinometostat. Preferably the epigenetic agent is GSK2879552, ORY1001, GSK-LSD1 or CC90011.

The epigenetic agent may be IMG-7289 (Bomedemstat) or N-[(2S)-5-{[(1R, 2S)-2-(4-fluorophenyl) cyclopropyl]amino}-1-(4-methylpiperazin-1-yl)-1-oxopentan-2-yl]-4-(1H-1,2,3-triazol-1-yl)benzamide, bis-tosylate salt.

The epigenetic agent may be an LSD1 inhibitor. As used herein, the term “LSD1 inhibitor” refers to a therapeutic agent that reduces, decreases, blocks or inhibits the expression or activity of LSD1. For example, the LSD1 inhibitor can block or disrupt the catalytic active site of LSD1. The LSD1 inhibitor can be, e.g., a selective LSD1 inhibitor or a non-selective LSD1 inhibitor. The LSD1 inhibitor can be a small molecule, an antibody, or an inhibitory nucleic acid. A non-exhaustive list of small molecule LSD1 inhibitors is provided in Table 7:

TABLE 7

| | Generic name or | |
|---|---|---|
| | name as used in | |
| Chemical name | clinical trials | CAS# |
| trans-2-Phenylcyclopropylamine hemisulfate salt | Tranylcypromine | 13492-01-8 |
| 1-(4-methylpiperazin-1-yl)-2-[[2-(4- | RN 1 | 1781835-13-9 |
| phenylmethoxyphenyl)cyclopropyl]amino]eth anone | dihydrochloride | |
| rel-N-[(1R,2S)-2-Phenylcyclopropy1]-4-Piperidinamine | GSK-LSD1 | 1431368-48-7 |
| hydrochloride (1:2) | | |
| 4-[4-[[[(1R,2S)-2-phenylcyclopropyl]amino]methy1]- | GSK2879552 | 1401966-69-5 |
| 1-piperidinyllmethyll-benzoic acid | | |
| rel-N1-[(1R,2S)-2-phenylcyclopropyl]-1,4- | ORY1001 | 1431326-61-2 |
| cyclohexanediamine, dihydrochloride | | |
| (R)-4-[5-(Pyrrolidin-3-ylmethoxy)-2-p-tolyl- | GSK690 | 2101305-84-2 |
| pyridin-3-yl]-benzonitrile Bis-TFA Salt | | |
| 3-Chloro-6-nitro-2-(trifluoromethyl)-4H-1-benzopyran-4-one | Namoline | 342795-11-3 |
| 1,15-bis IN543,3-(diphenyl)propyll-N1-biguanido}-4,12- | Cpd 2d | |
| diazapentadecane | | |
| (1R,25)-rel-2[3,5-Difluoro-2- | 52101 | 1239262-36-2 |
| (phenylmethoxy)phenylicycloprpanamine hydrochloride | | |
| 4′-[(1R,2S)-2-aminocyclopropy1]41,1′-biphenyl]-3-ol | 0G-L002 | 1357302-64-7 |
| 3-(4-morpholinylsulfony1)-benzoic acid (2E)-2-[1-(5-chloro-2- | SP2509 | 1423715-09-6 |
| hydroxyphenypethylidenelhydrazide | | |
| Methyl-3-(4-(4- | CBB1007 | 1379573-92-8 |
| carbamimidoylbenzoyl)piperazine-1-carbony1)-5-((4- | | |
| carbamimidoylpiperazin-1-yl)methyl)benzoate | | |
| N-[(2S)-5-{[(1R,2S)-2-(4- | IMG-7289 | |
| fluorophenyl)cyclopropyll amino}-1-(4-methylpiperazin-I-y 1)-1- | | |
| oxopentan-2-y11-4-(1H-1,2,3-triazol-1-yObenzamide, bis-tosylate | | |
| salt | | |

The LSD1 inhibitor may be any small molecule LSD1 inhibitor identified in Table 7. The LSD1 inhibitor may be an LSD1 inhibitor known in the art e.g., in US 20150225401, US 20170129857, US20170281567, US20170281566, US20170183308, US20170283397, US20170209432, US20170044101, U.S. Pat. Nos. 9,493,442, 9,346,840, WO/2016/007736, WO/2016/161282, US 20160009711, Eu et al., Advances toward LSD1 inhibitors for cancer therapy, Future Medicinal Chemistry, vol. 9, no. 11 (2017), WO2019/083971 and WO2021/118996; each of which is incorporated herein by reference in its entirety.

The kinase inhibitor may inhibit a kinase selected from the group consisting of mitogen-activated protein kinase kinase 1/2 (MEK1 or MEK2), mammalian target of rapamycin kinase (mTOR), protein kinase C (PKC), fins like tyrosine kinase 3 (FLT-3), phosphoinositide 3-kinase (PI3K) or casein kinase 2 (CK2), preferably wherein the kinase is MEK1 or MEK2, their downstream and upstream effectors ARAF, BRAF, CRAF, ribosomal S6 kinases (RPS6KA1, RPS6KA2, RPS6KA3), extracellular signal-regulated kinases 1 and 2, (ERK1 and ERK2, gene names MAPK3 and MAPK1, respectively).

The kinase inhibitor may be selected from trametinib, midostaurin, pictilisib, torin 1 and silmitasertib. Preferably the kinase inhibitor is trametinib.

The epigenetic agent and/or kinase inhibitor is preferably administered to a patient in a “therapeutically effective amount”, this being sufficient to show benefit to the patient and/or to ameliorate, eliminate or prevent one or more symptoms of a disease. As used herein, “treatment” includes any regime that can benefit the patient.

Dosages of epigenetic agent and/or kinase inhibitor for use in the present invention can vary between wide limits, depending upon the stage of the cancer, the age and condition of the individual to be treated, etc. and a physician will ultimately determine appropriate dosages to be used. This dosage can be repeated as often as appropriate. If side effects develop the amount and/or frequency of the dosage can be reduced, in accordance with normal clinical practice.

The epigenetic agent and/or kinase inhibitor may preserve normal stem/progenitor cells while inducing apoptosis in differentiated and/or cancer cells.

In certain configurations:
(i) the cancer is acute myeloid leukaemia, optionally wherein the cancer is classified as being a FAB M4, M5 or M6 subtype acute myeloid leukaemia or as KMT2A rearranged (KMT2Ar) acute lymphoblastic leukaemia (ALL);
(ii) the epigenetic agent is ATRA or inhibits LSD1, optionally wherein the epigenetic agent is GSK2879552; and
(iii) the kinase inhibitor inhibits MEK, optionally wherein the kinase inhibitor is trametinib.

In certain configurations the cancer comprises:
(a) cells expressing one or more of the following cluster of differentiation proteins: CD11b, CD64, CD14, CD117, CD16 and/or CD15; and/or
(b) higher activity of AKT1, PI3KCA and/or MAPK1/3 signalling pathways and/or higher phosphorylation of one or more MAPK and PI3K/mTOR pathway activation markers, such as PAK1 at T219, MEK2 at S23, MAPK3 at T202/T207/Y204 and AKT1S1 (PRAS40) at S183, relative to a threshold level per marker; and/or
(c) Ras mutations or increased protein expression of one or more targets of Ras downstream targets, such as MP2K5, (MEK1 (MAPK2K1), MEK2 (MAP2K2), MAPK1, MAPK3, and ribosomal S6 kinase relative to a threshold level per marker.

In certain configurations the cancer comprises cells expressing one or more of the following cluster of differentiation proteins: CD11b, CD64, CD14, CD117, CD16 and/or CD15 and:
(i) if the cancer comprises cells expressing CD11b and/or CD64 then the kinase inhibitor is trametinib; or
(ii) if the cancer comprises cells expressing CD117, CD16 and/or CD15 then the kinase inhibitor is silmitasertib.

When the cancer comprises cells expressing CD11b and CD64 then the kinase inhibitor may be trametinib.

When the cancer comprises cells expressing CD117, CD16 and CD15 then the kinase inhibitor may be silmitasertib.

“About” and “approximately” shall generally mean an acceptable degree of error for the quantity measured given the nature or precision of the measurements. Typically, exemplary degrees of error are within 20 percent (%), preferably within 10%, and more preferably within 5% of a given value or range of values.

Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

Preferred features for the second and subsequent aspects of the invention are as for the first aspect of the invention mutatis mutandis. It will be appreciated that all embodiments described herein are considered to be broadly applicable and combinable with any and all other consistent embodiments, as appropriate. Such combinations are considered to fall within the scope of the present invention.

Further advantages of the invention are described below.

Kinase inhibitors can be highly effective for treating some tumour types, but most cancer patients fail to respond or become resistant to these targeted drugs. Intrinsic resistance to kinase inhibitors has been ascribed to lack of target activity within cancer cells or to the existence of pro-survival pathways that compensate for target inhibition. Thus, overall signalling circuitry determines the extent by which cancer cells respond to kinase targeted drugs. Here, we show that intrinsic resistance to kinase inhibitors may be overcome by coercing kinase networks into acquiring topologies tolerant to drug sensitivity. Using acute myeloid leukaemia (AML) as a model, we found several antagonists of chromatin modifying enzymes that sensitised cancer cells to kinase inhibitor treatments. Of these, we confirmed that the LSD1/KDM1A inhibitor GSK2879552 rewires kinase signalling in AML cells, which, as a result, get primed for trametinib (MEKi) treatment. Mechanistically, LSD1i forces tumour cells to acquire MEKi sensitivity by inducing a PI3K/AKT to MEK/MAPK signalling switch, by dampening down global kinase signalling that could compensate for MEK inhibition, and by blocking feedback loops. Overall, our study uncovers means to modulate kinase network circuitry and highlights a strategy for overcoming therapeutic resistance to kinase targeted drugs.

Kinase inhibitors are effective in small patient populations due to drug resistance. We have found that LSD1 regulates the activity of RAS/MAPK and PI3K signalling pathways. Pre-treatment of cells with LSD1i reverses intrinsic resistance to MEKi in AML but not all cases respond. We have identified biomarkers of responses to LSD1i followed by MEKi sequential treatment in AML. Therefore, the invention advantageously provides a treatment that uses LSD1i and MEKi in tandem for patients positive for biomarkers that predict responses.

The results of clinical trials suggest that MEK and LSD1 inhibitors are unlikely to be of significant clinical utility as single agents in unselected AML patient populations. MEKi alone has low efficacy and LSD1 inhibitor GSK-2879552 has side effects.

Our study provides a rationale for a therapeutic strategy in which pre-treatment with agents such as ATRA or specific epigenetic inhibitors is first used to coerce cancer cells into acquiring signalling topologies associated with sensitivity to inhibitors of kinases downstream of growth factor receptors. This would reinforce the effects of kinase blockade by preventing cancer cells from activating compensatory pathways that allow tumours to escape from therapy. Indeed, we found that LSD1i treatment blunts the activity of kinase pro-survival pathways (such as PI3K-AKT) that could compensate for MEK inhibition. This strategy differs from classical synthetic lethality approaches because, instead of co-treatments with two or more drugs, it aims to create new pathway dependencies by pre-treating tumours with agents that change their biochemical circuitry, thus producing phenotypes susceptible to subsequent treatments with targeted drugs.

The strategy is supported by the inventors have demonstration in vitro on retrospective tumour samples that an epigenetic inhibitor such as LSD1 (lysine specific demethylase 1 inhibitor) can induce a signalling switch that effectively dampens the kinase pathways except the one the second drug of use depends on i.e. compensatory kinase pathways are switched off. The second drug, e.g. a MEK inhibitor (MEKi), is then used to treat the tumour which has an effective tumour cell killing effect.

The present invention will now be described by way of reference to the following Examples and accompanying Drawings which are present for the purposes of illustration only and are not to be construed as being limiting on the invention.

### Example 1—Kinase Network Topologies are Associated with Haematopoietic Cell Differentiation Markers in Primary Acute Myeloid Leukemia

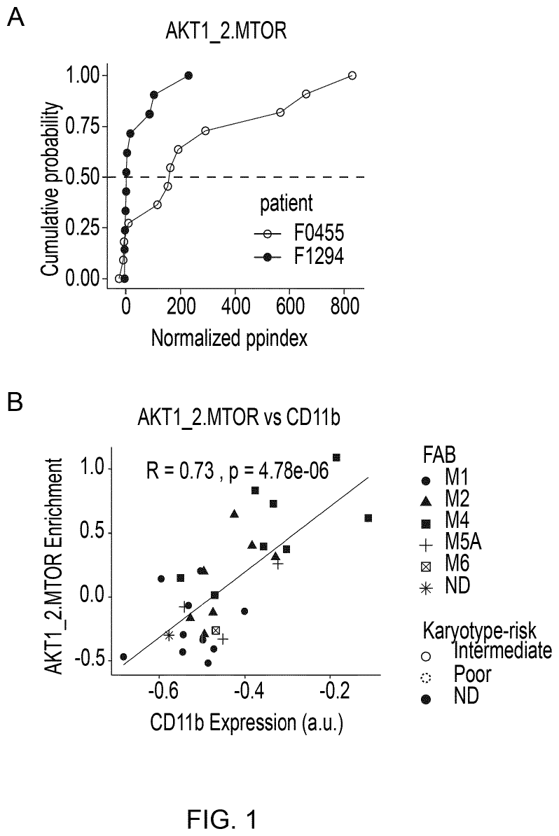

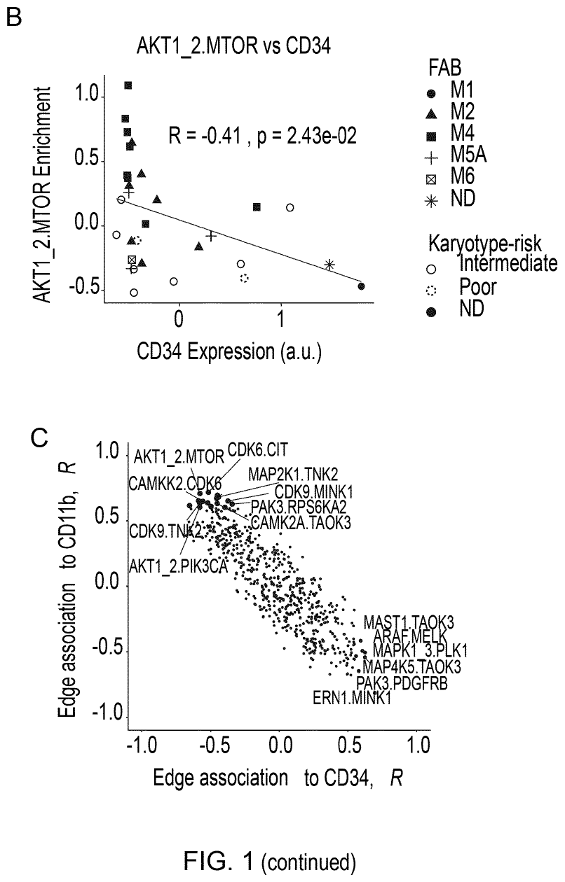

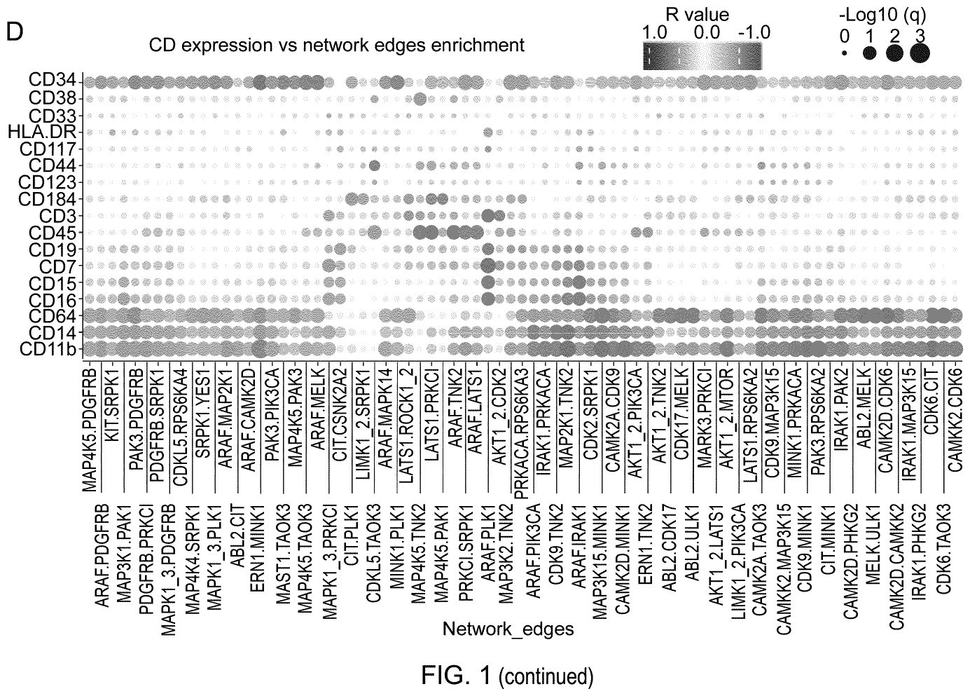

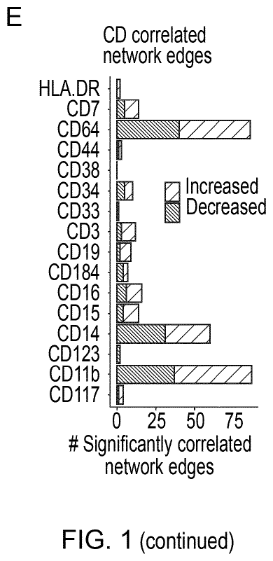

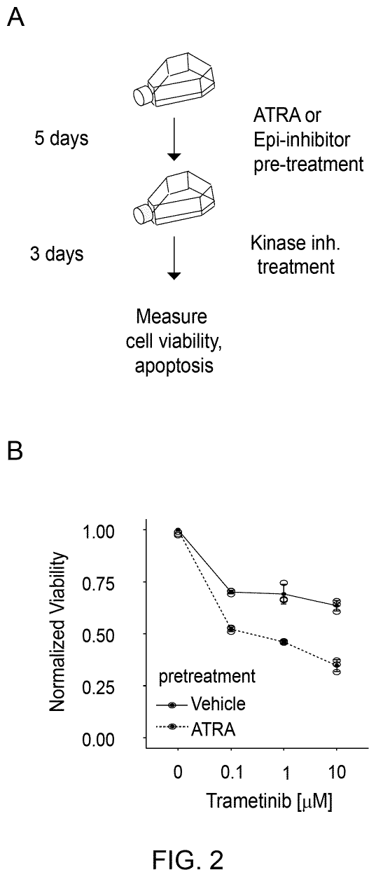

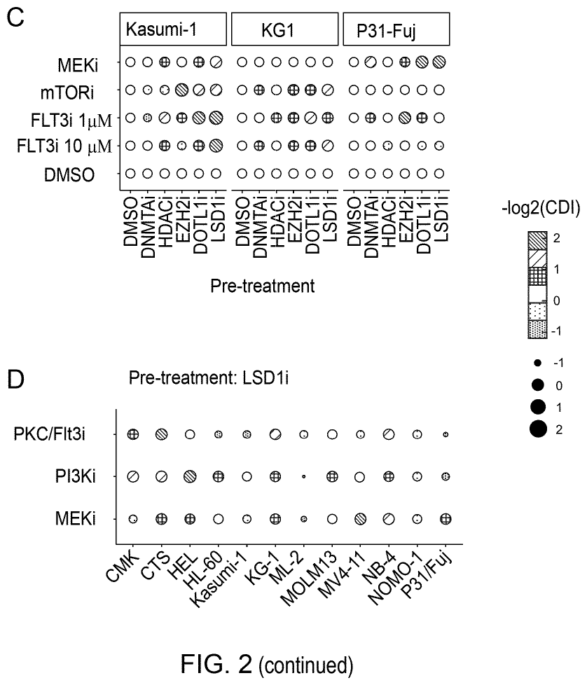

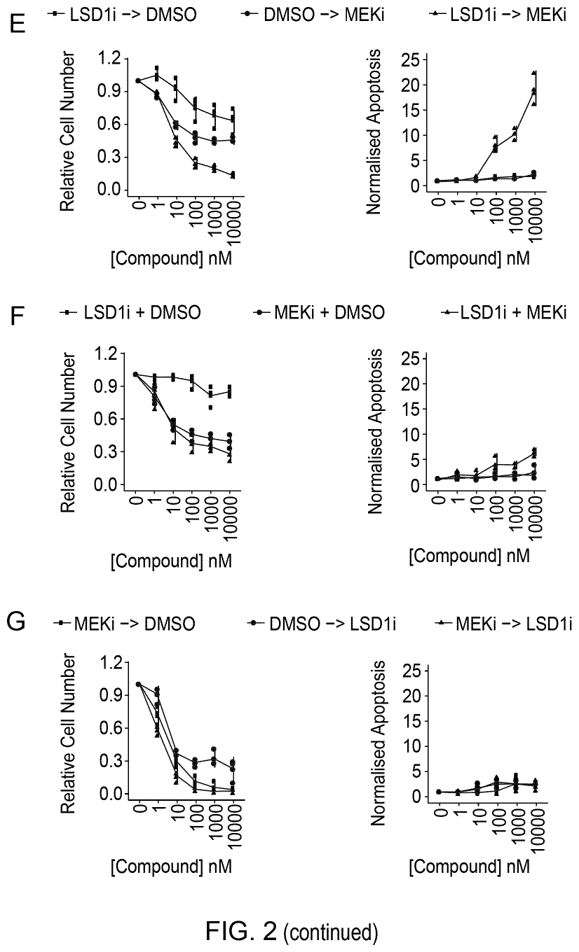

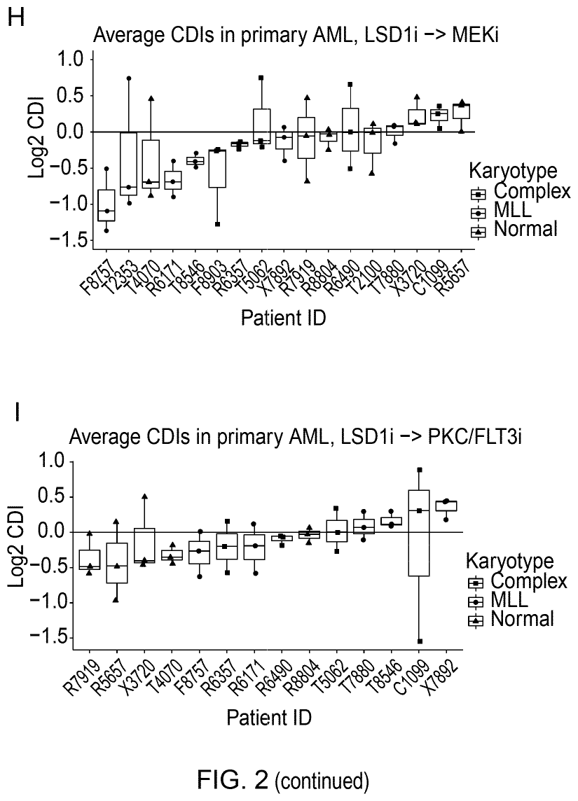

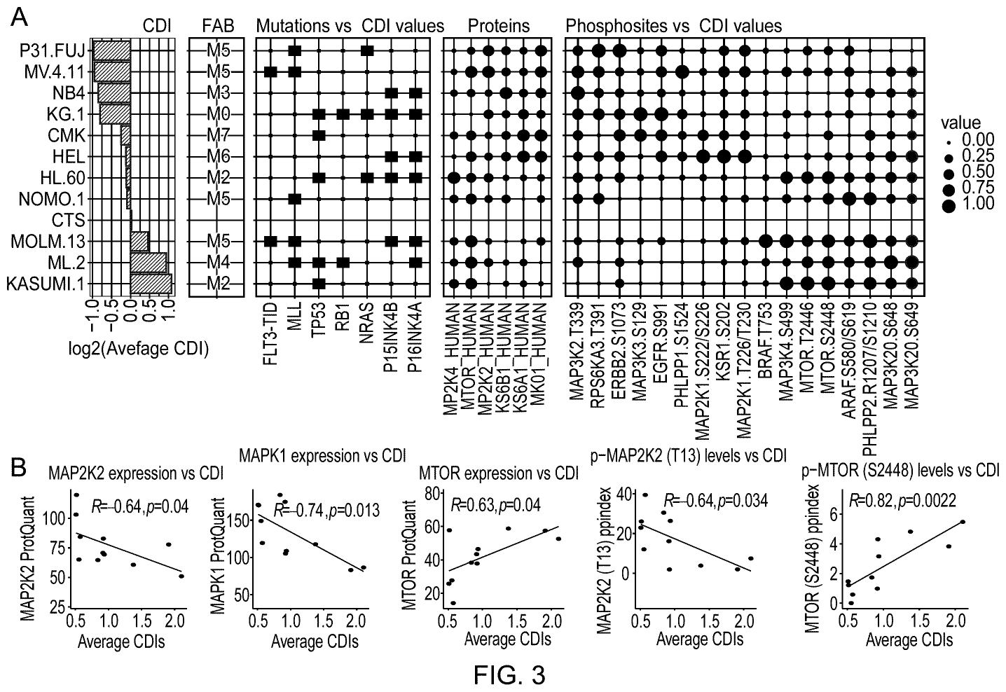

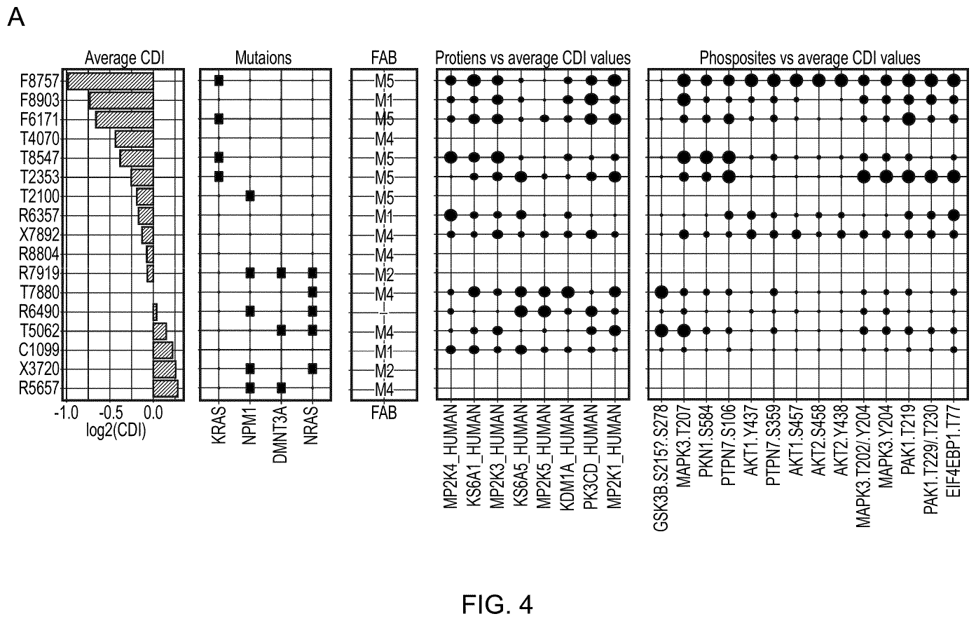

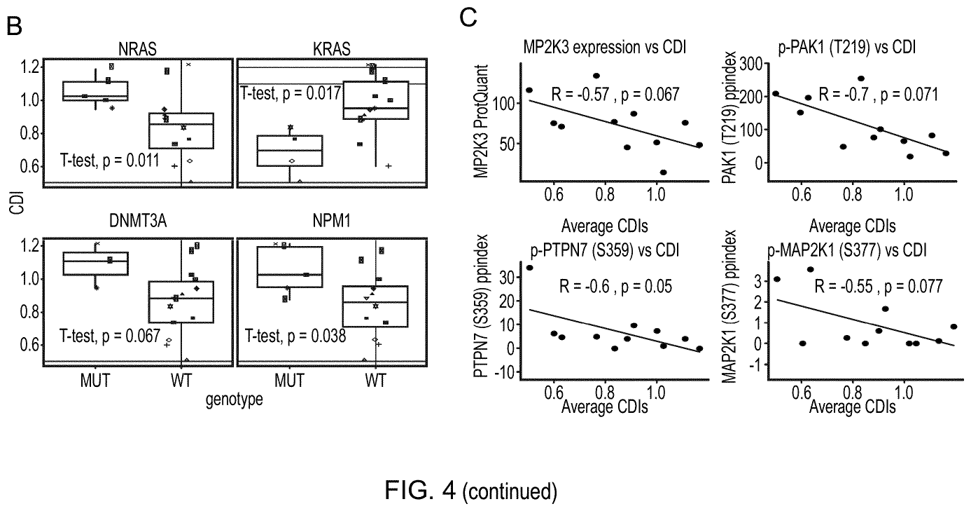

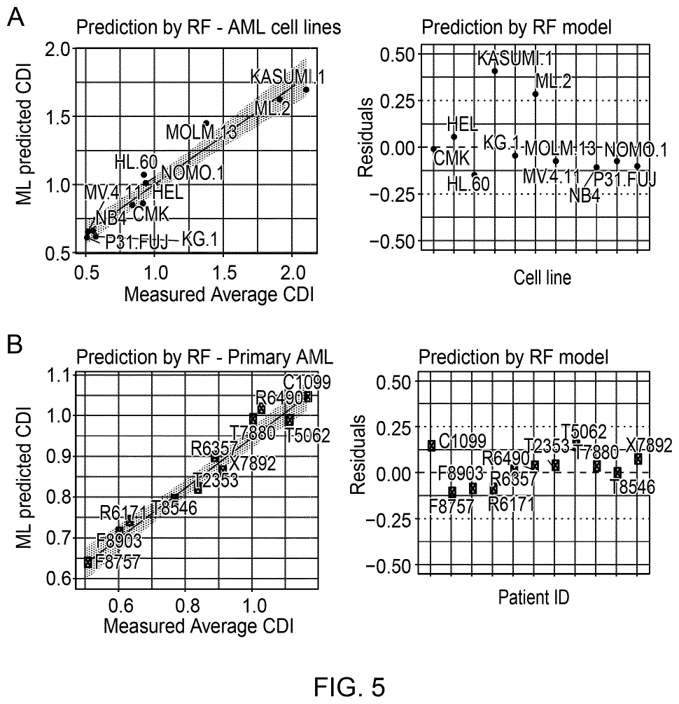

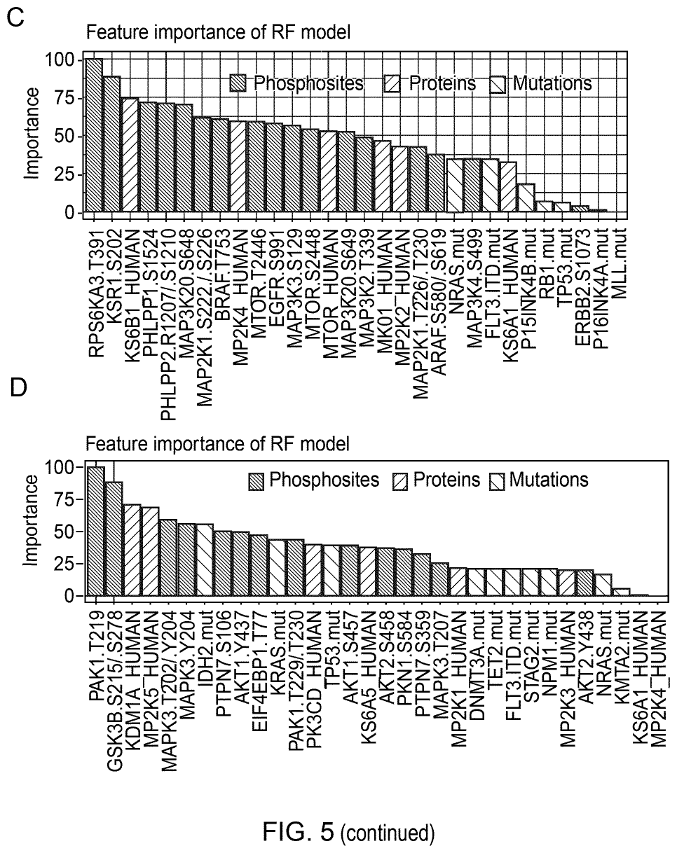

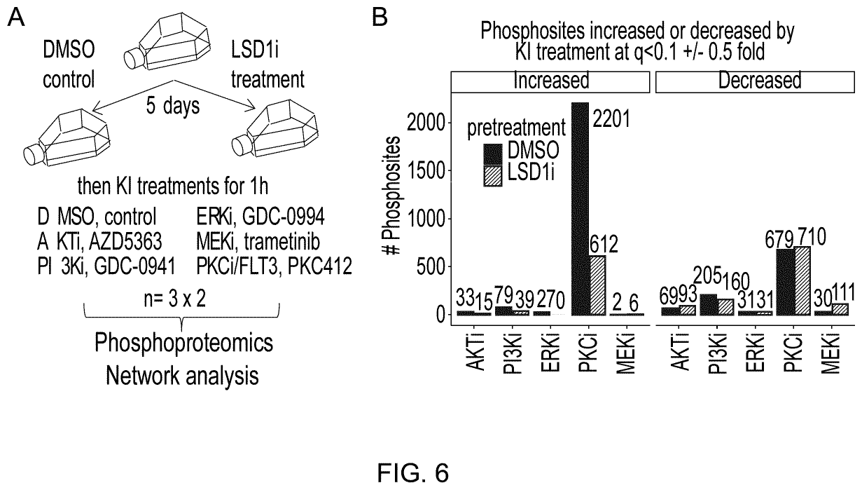

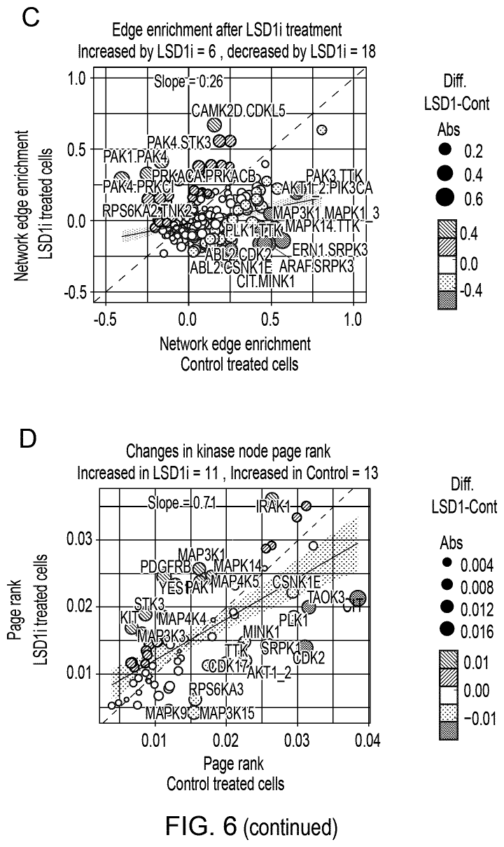

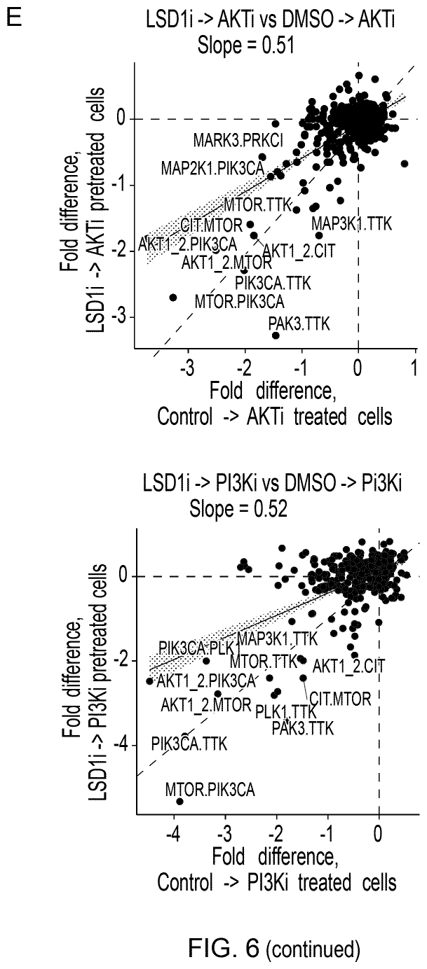

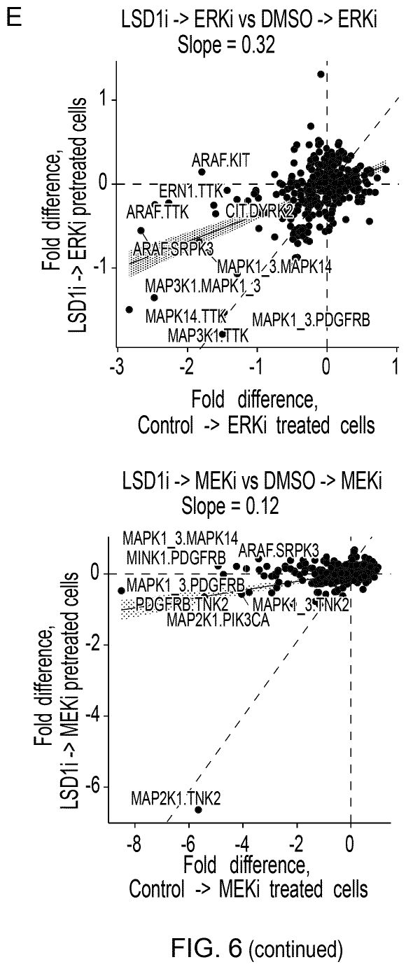

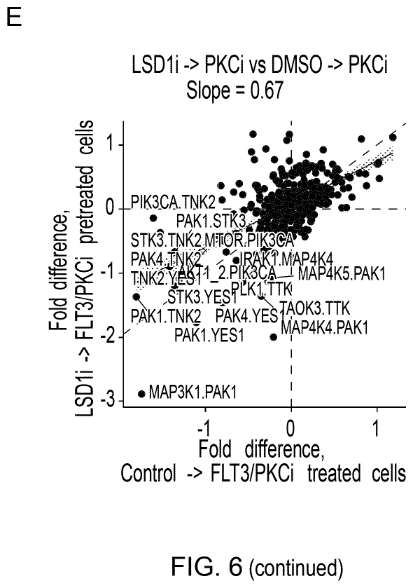

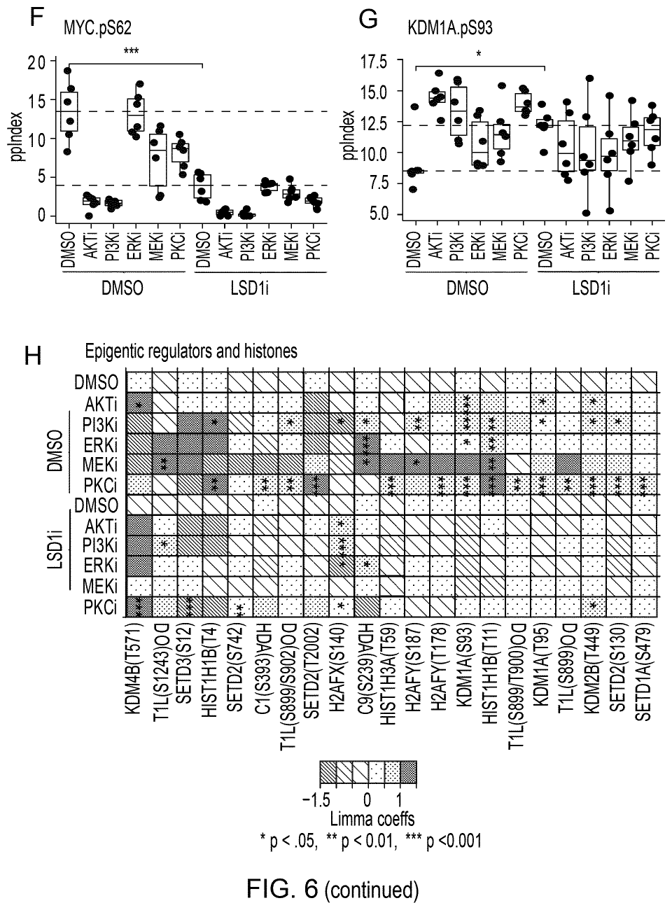

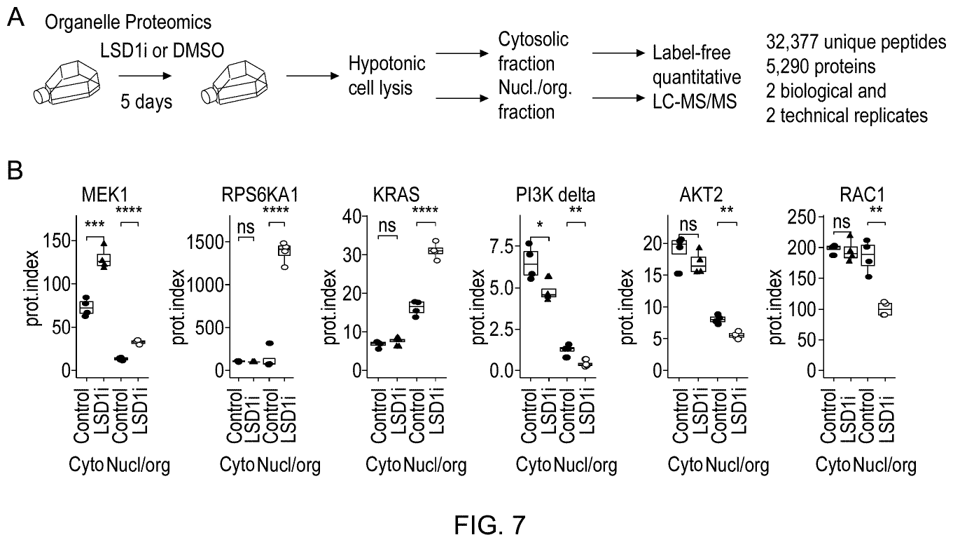

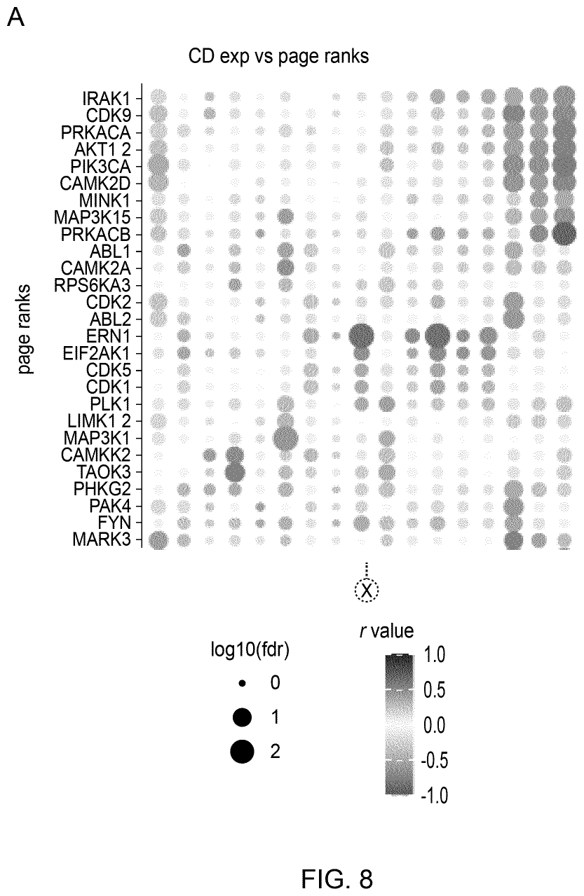

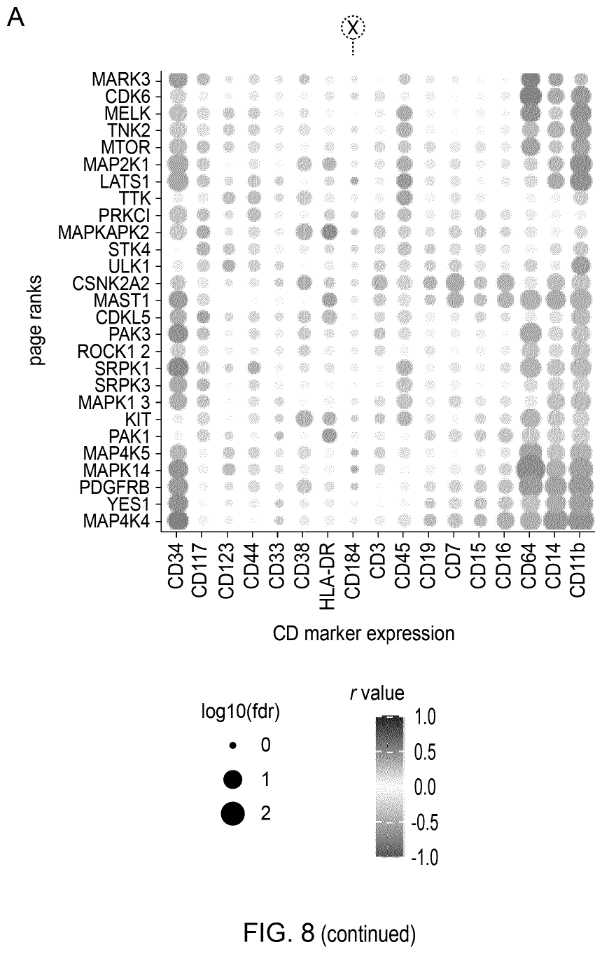

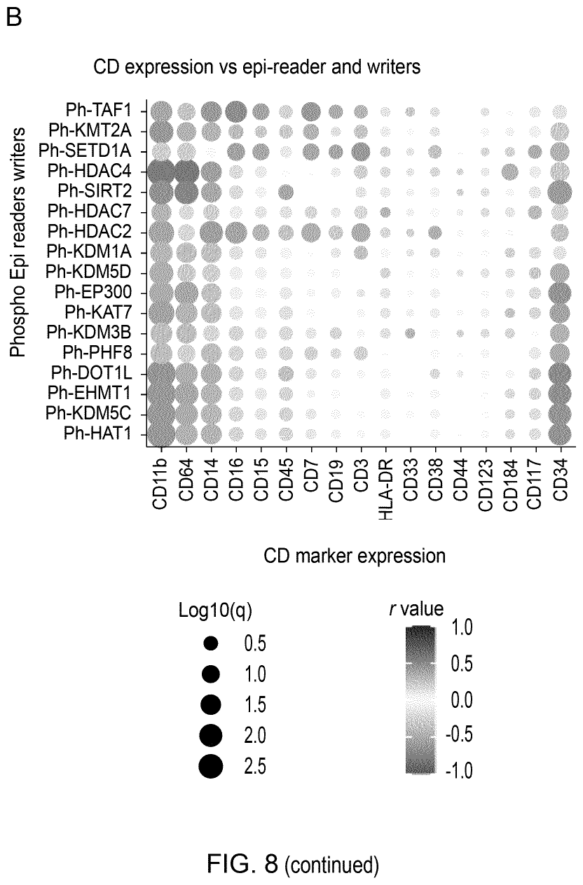

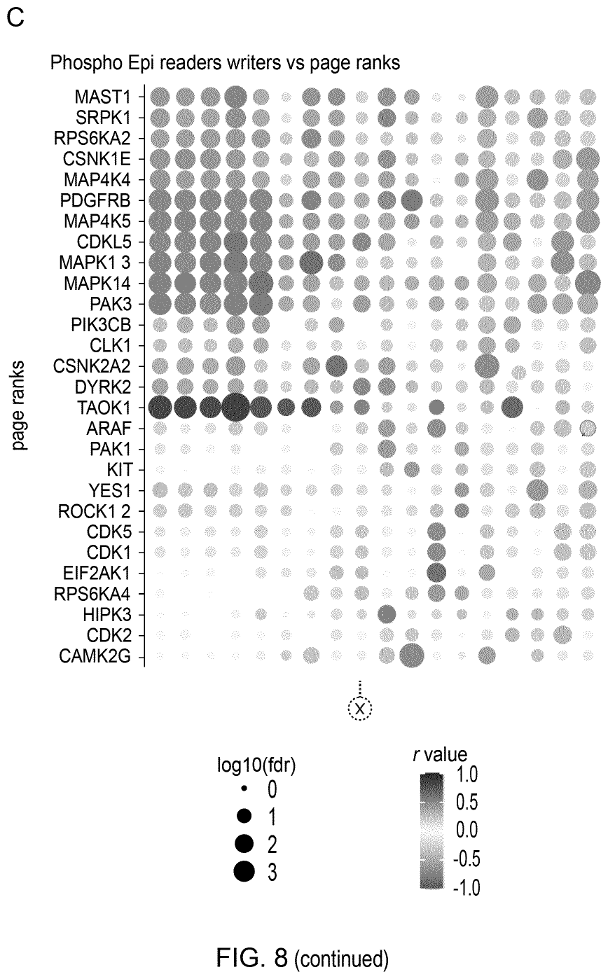

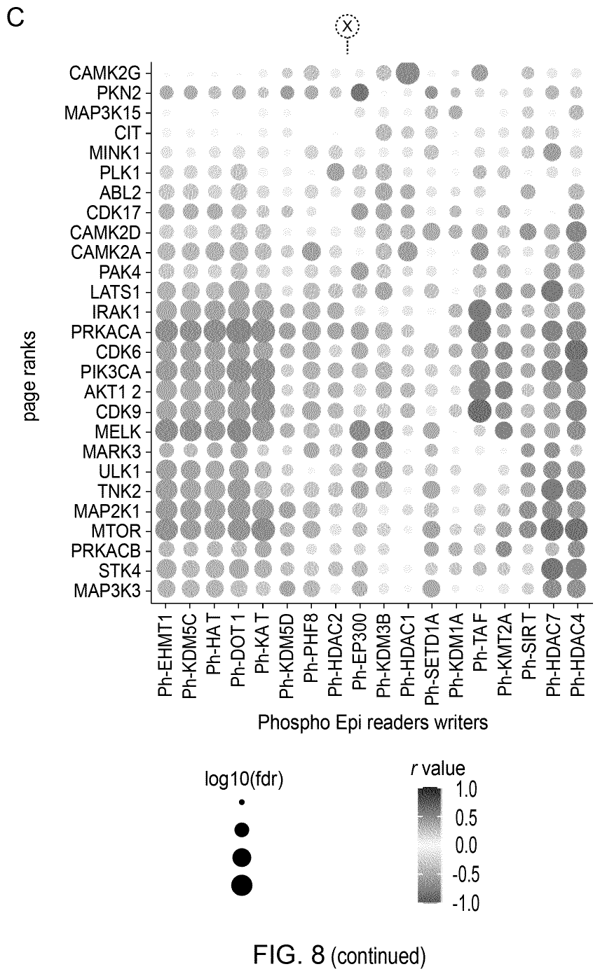

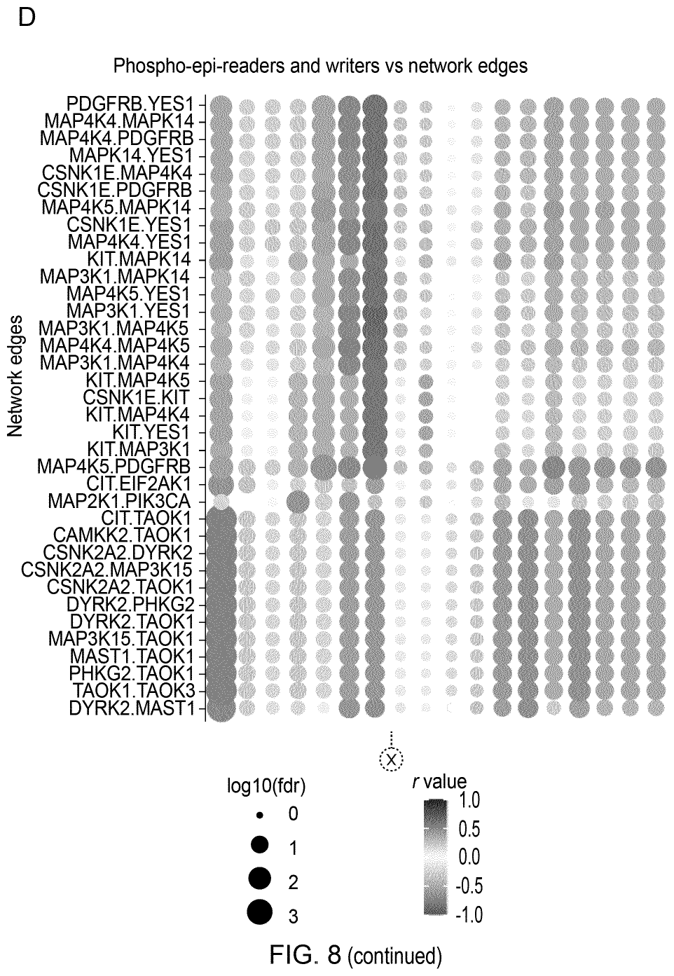

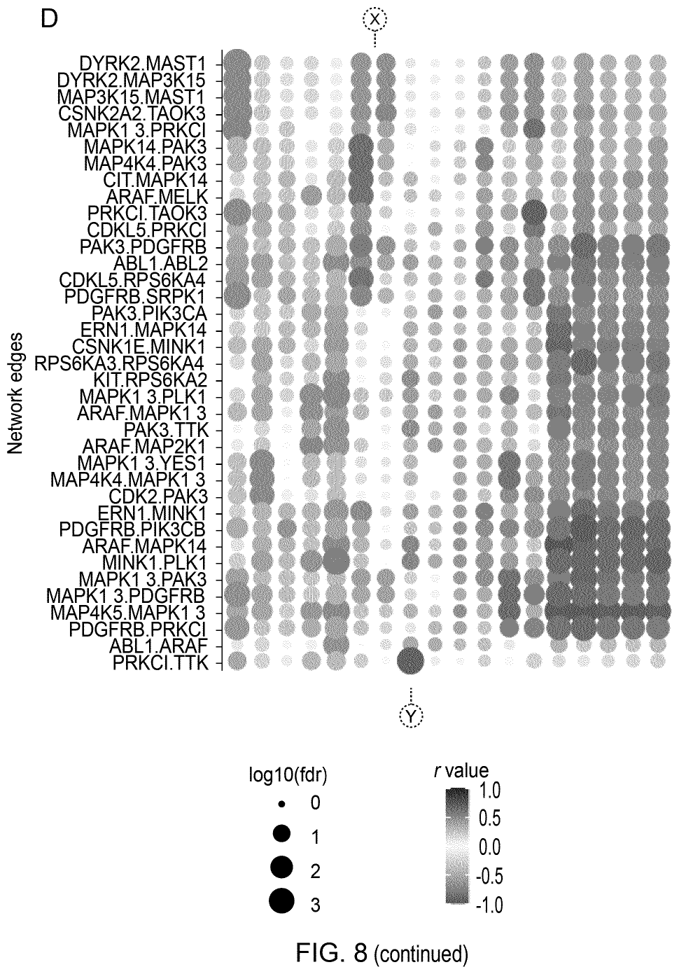

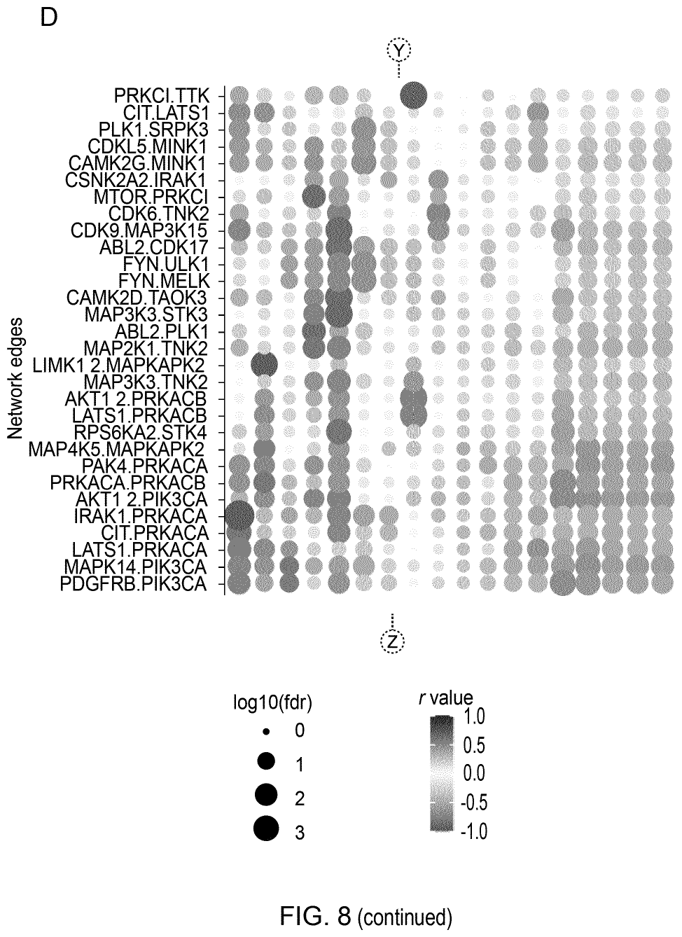

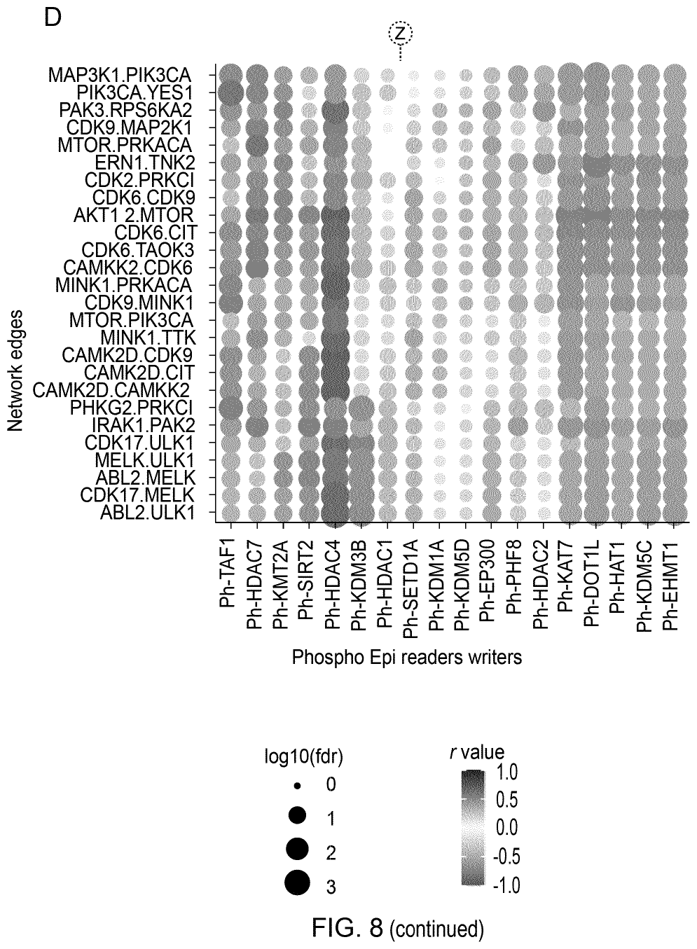

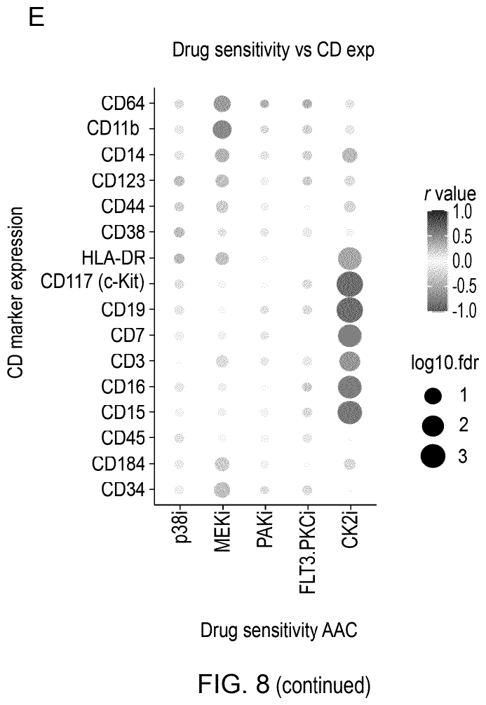

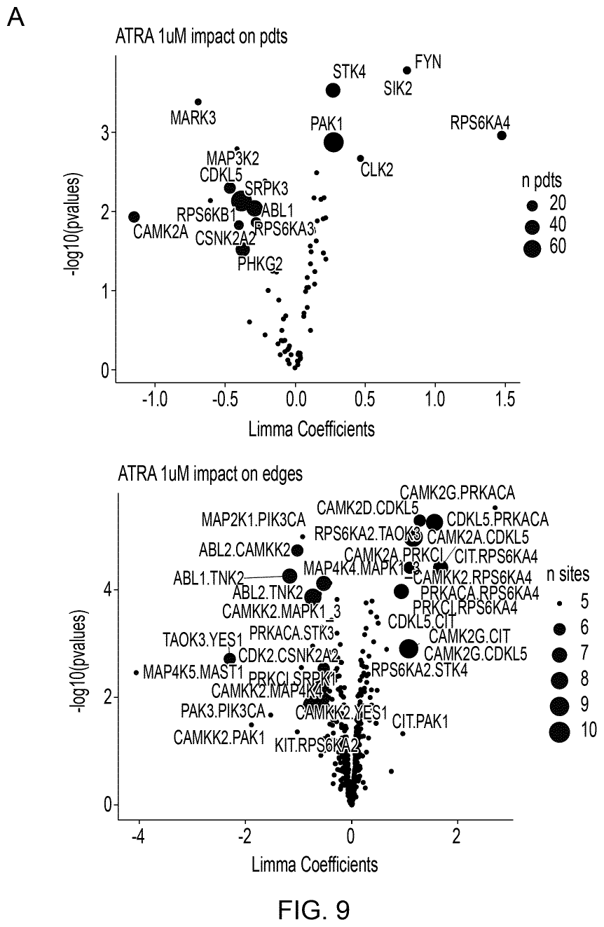

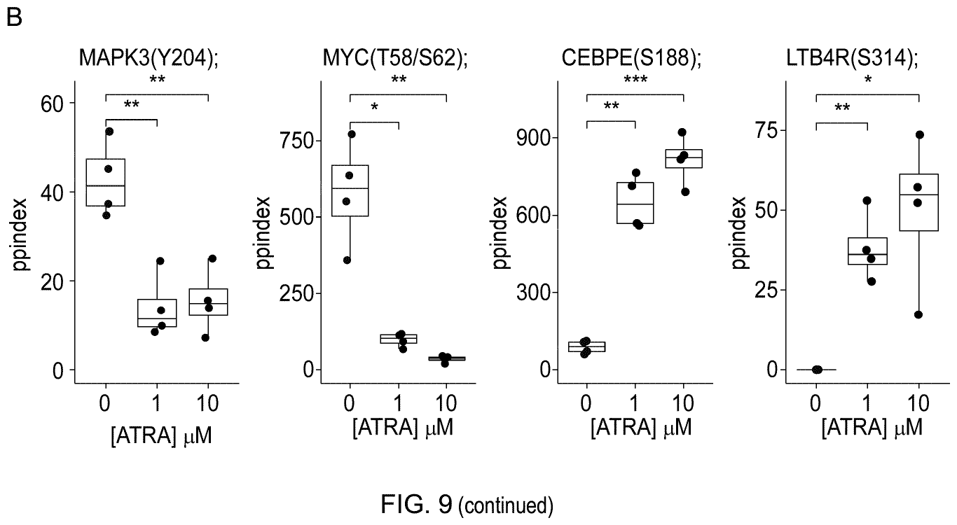

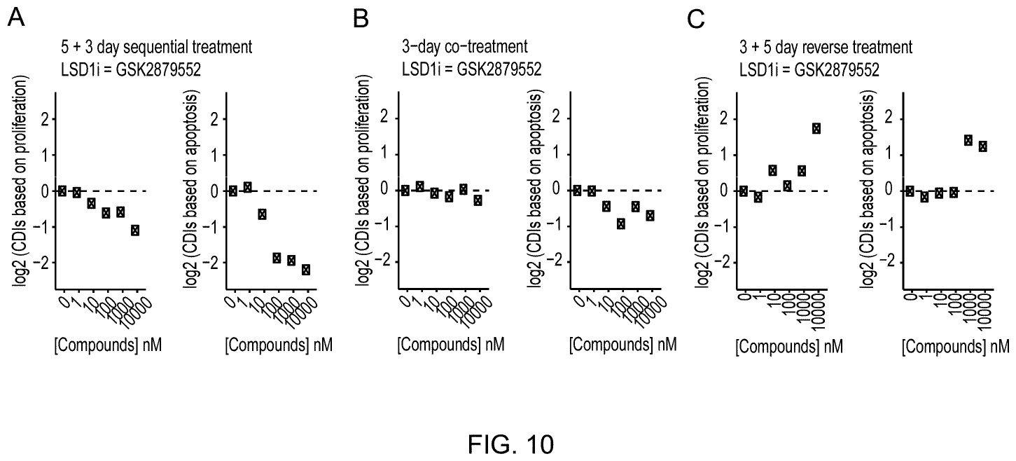

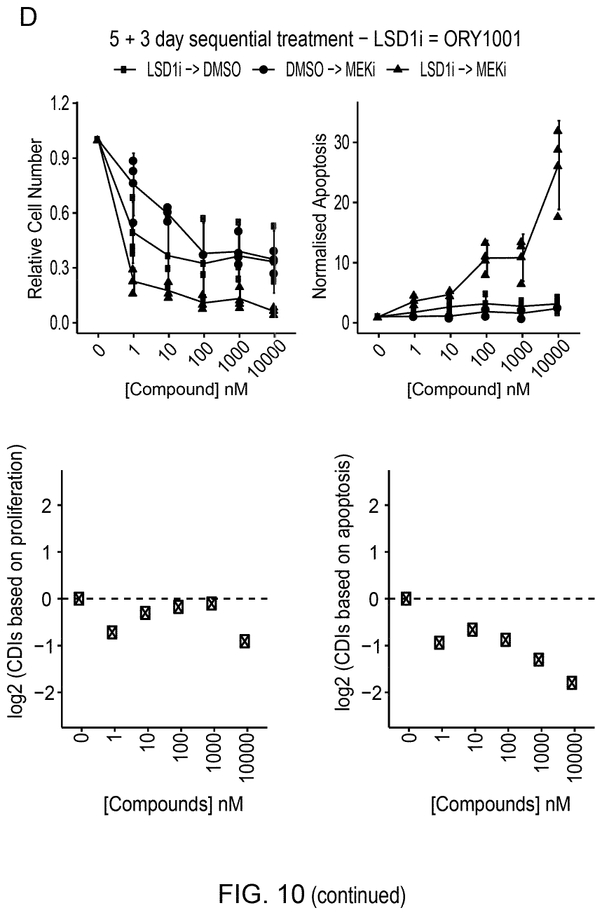

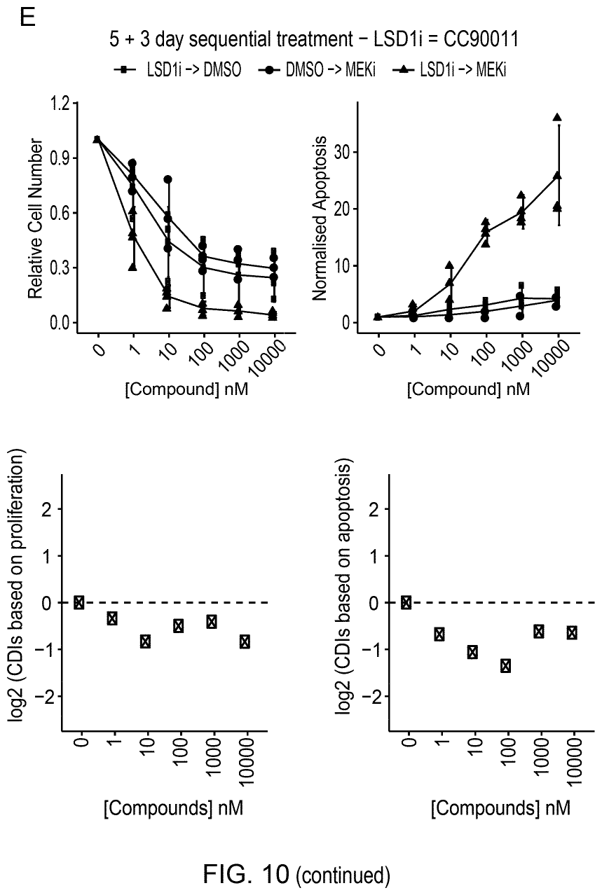

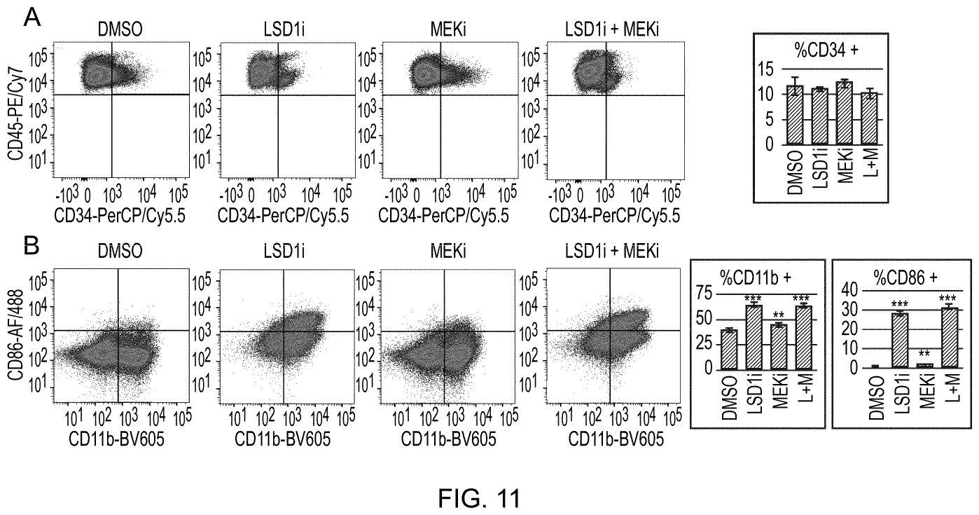

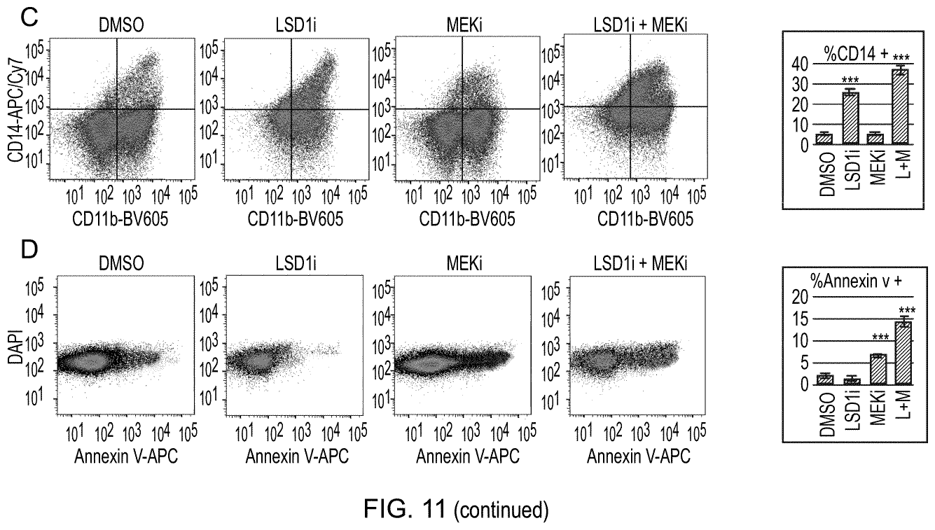

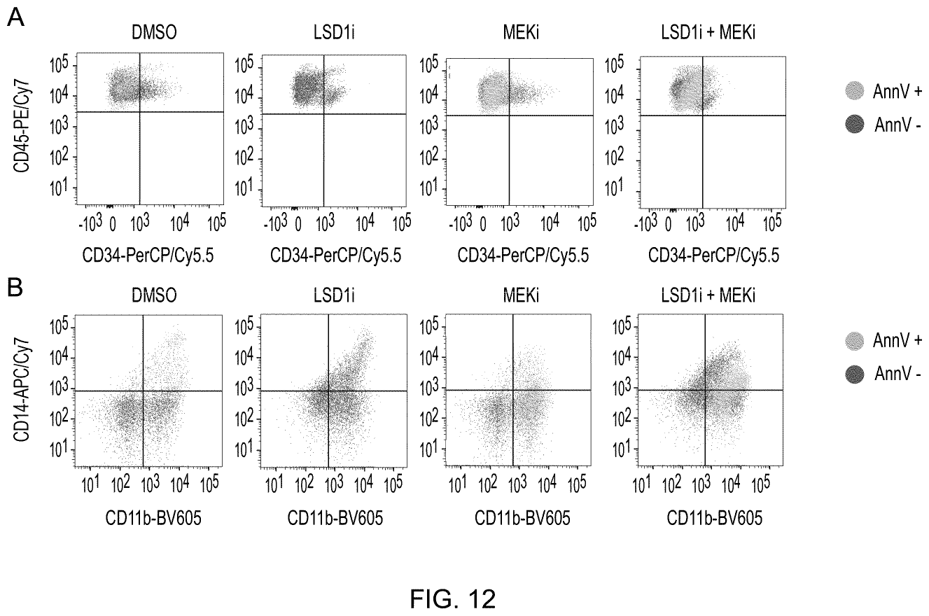

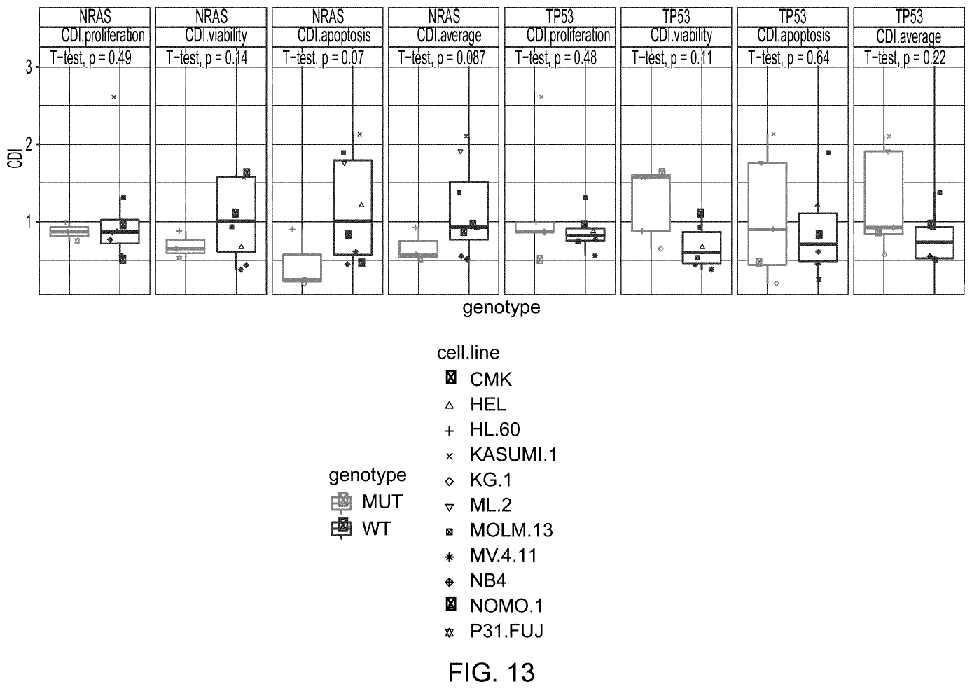

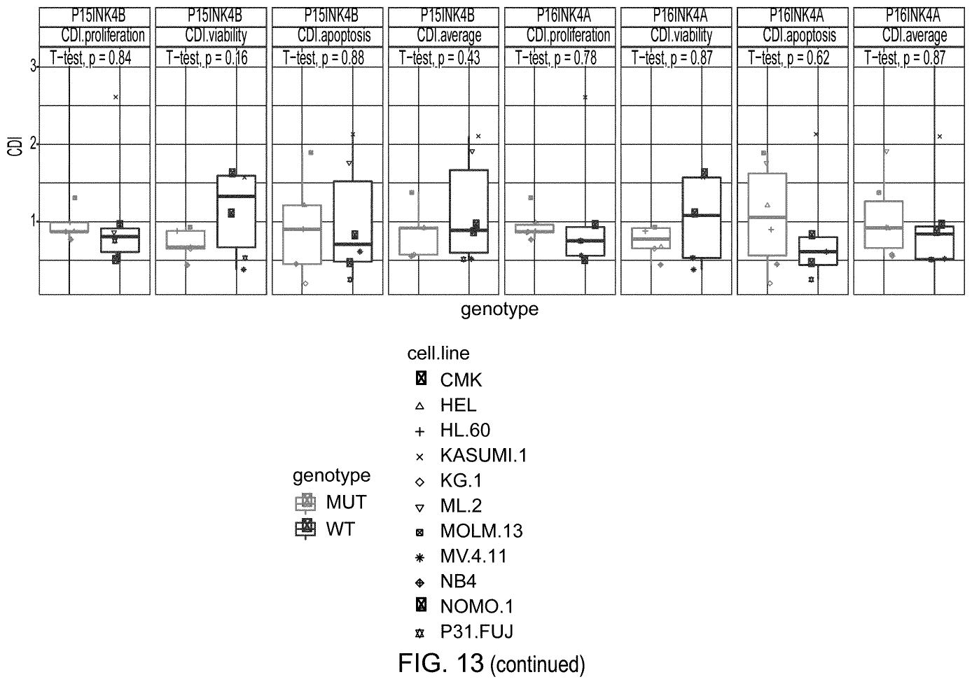

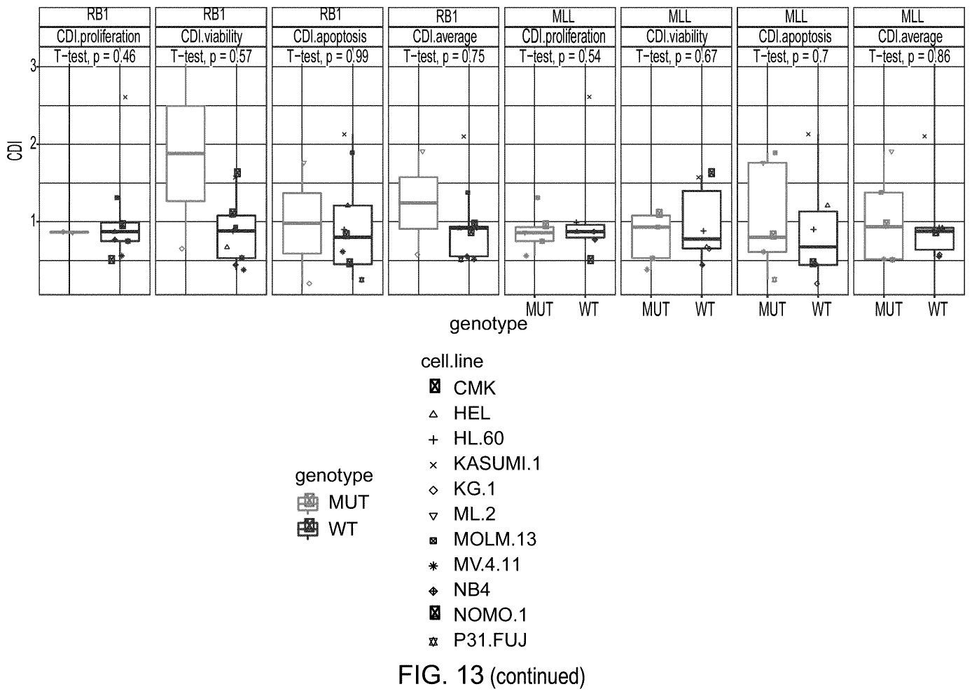

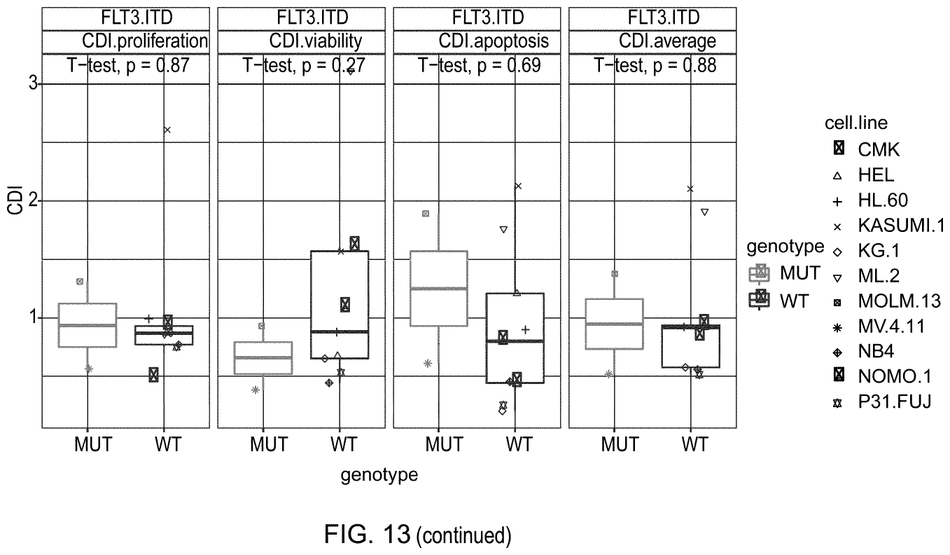

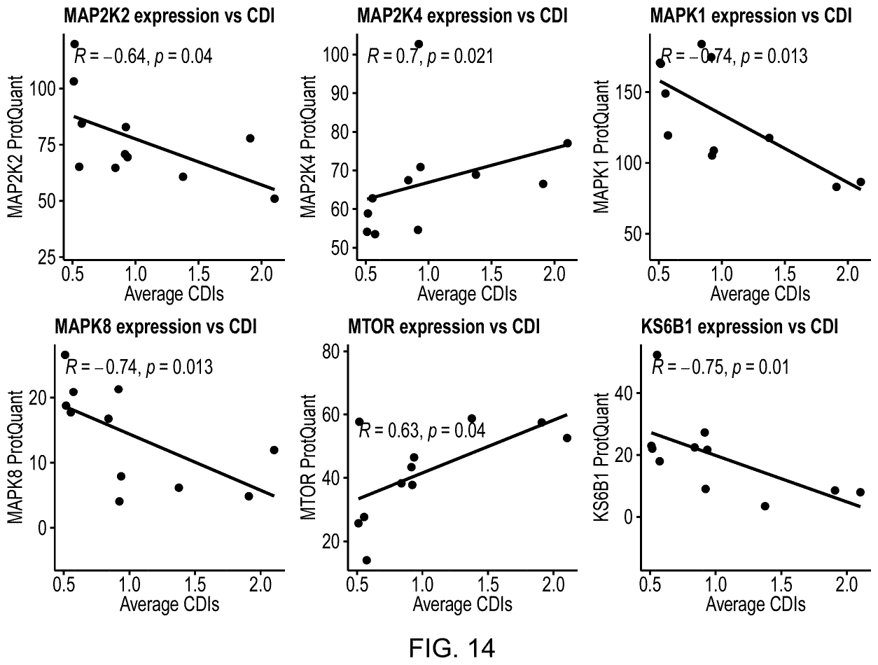

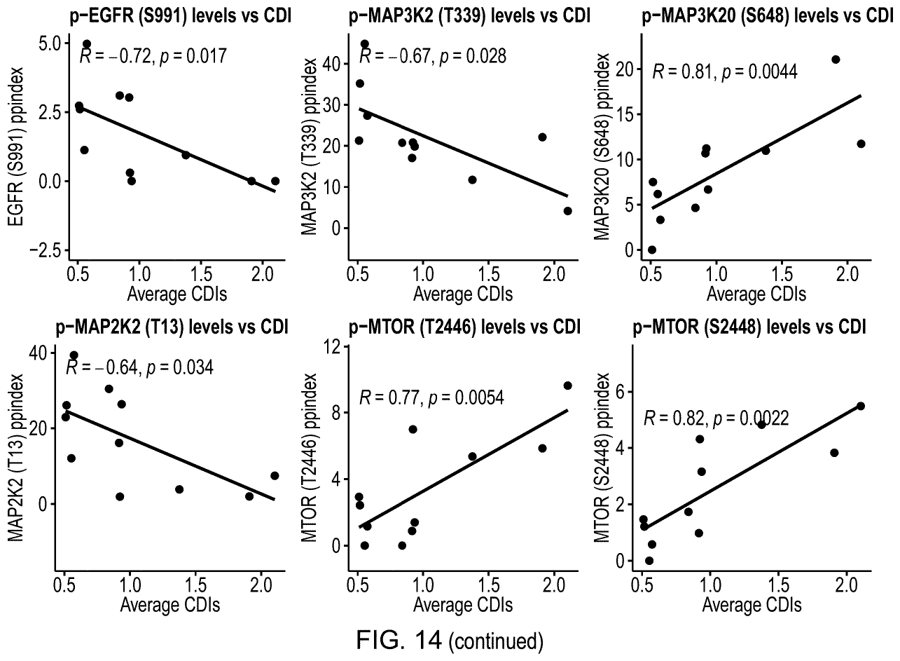

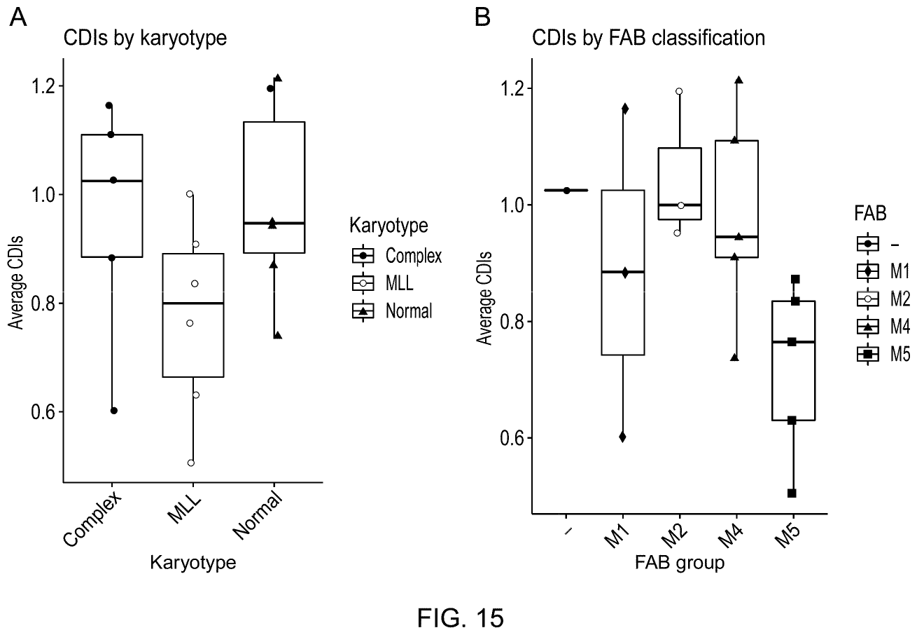

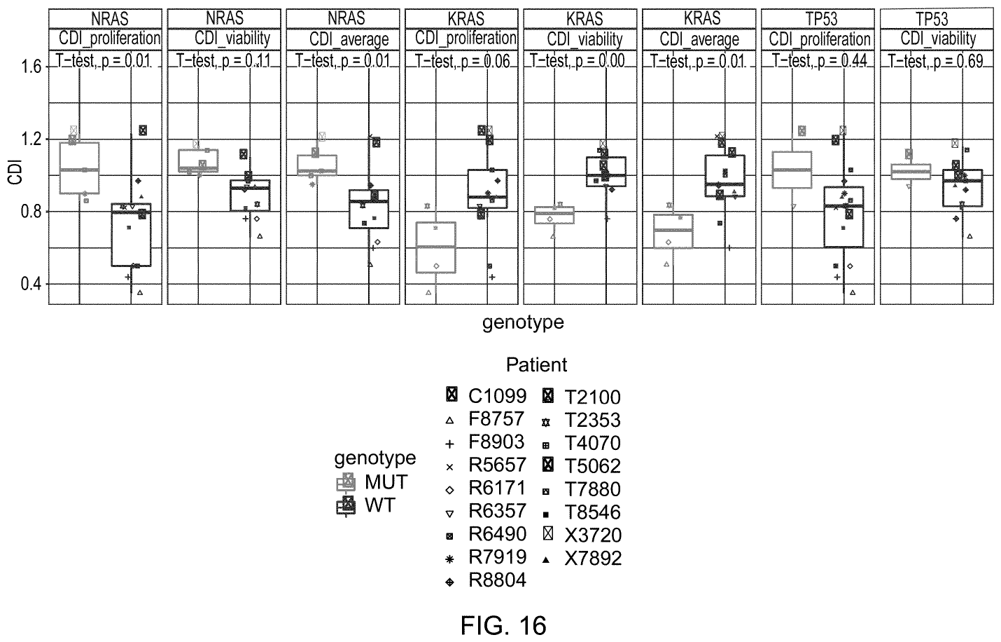

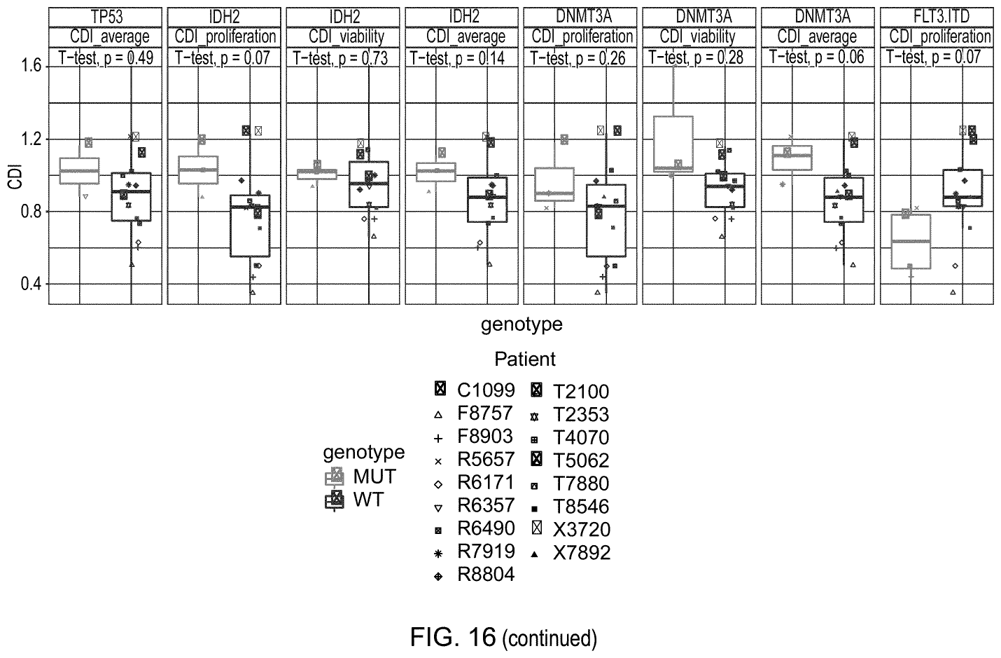

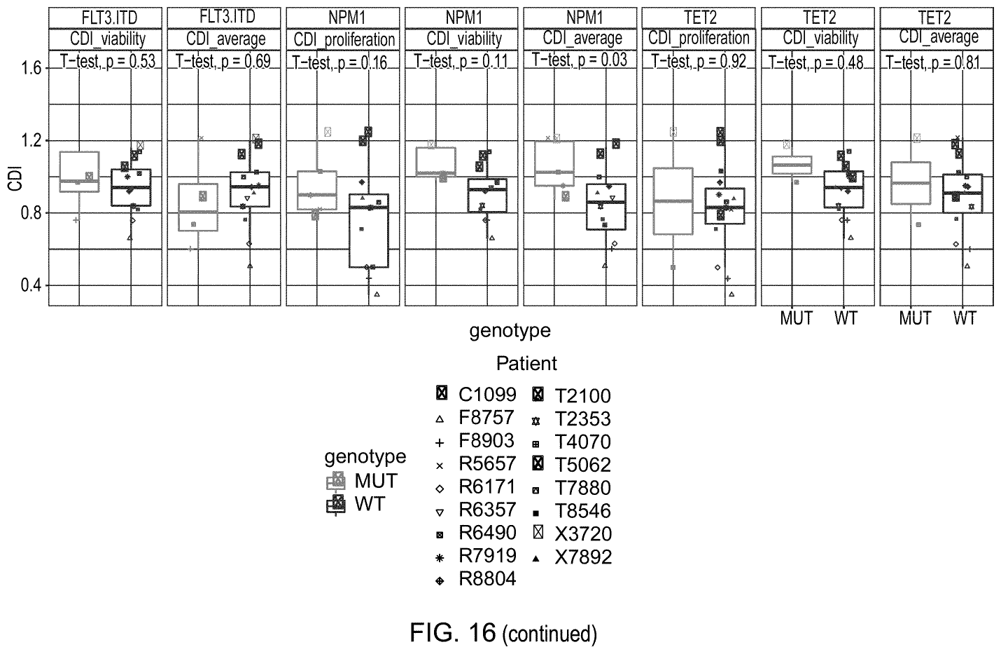

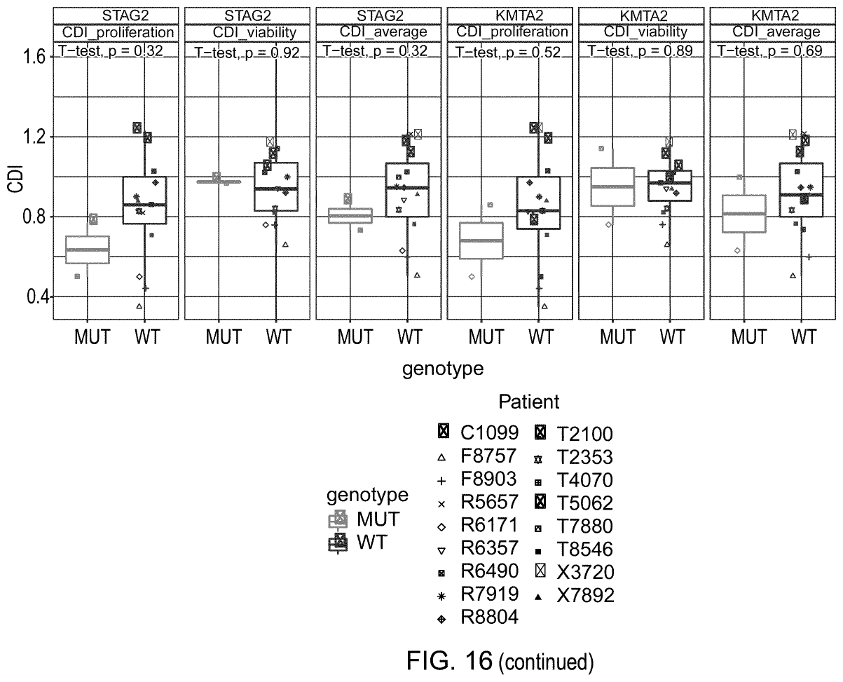

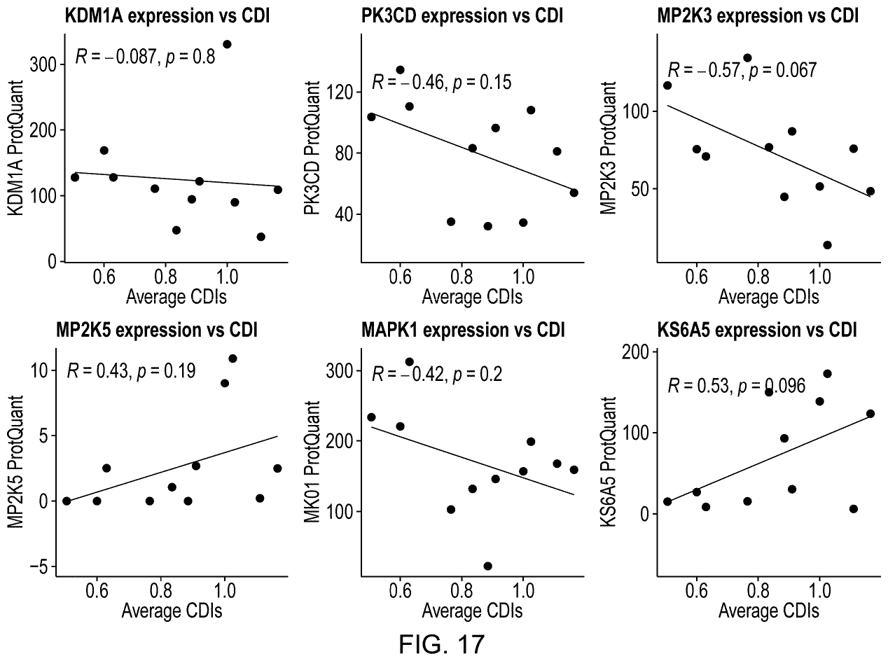

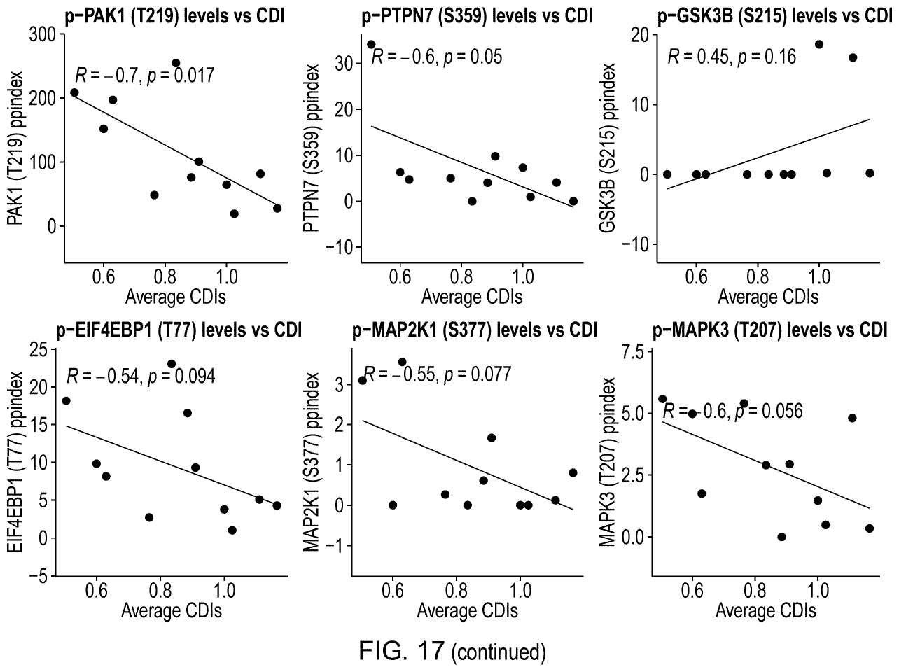

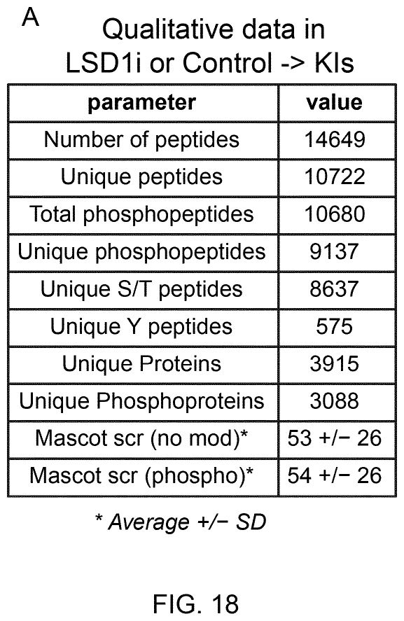

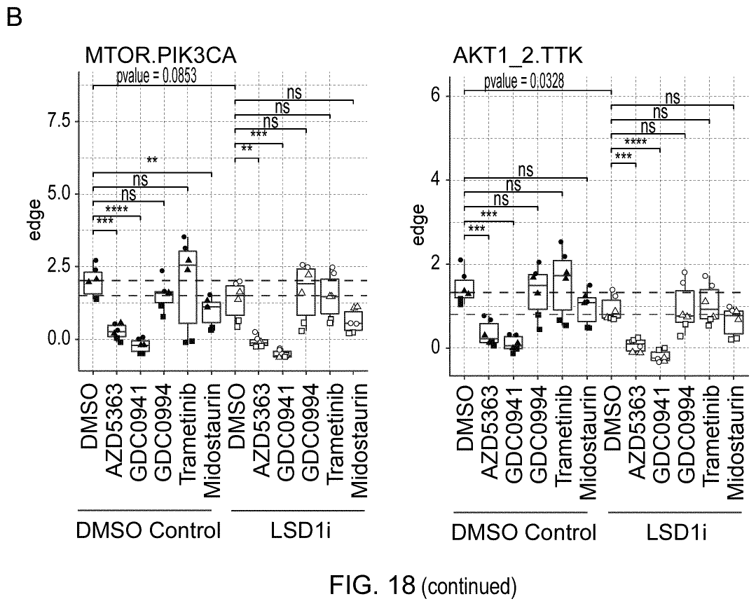

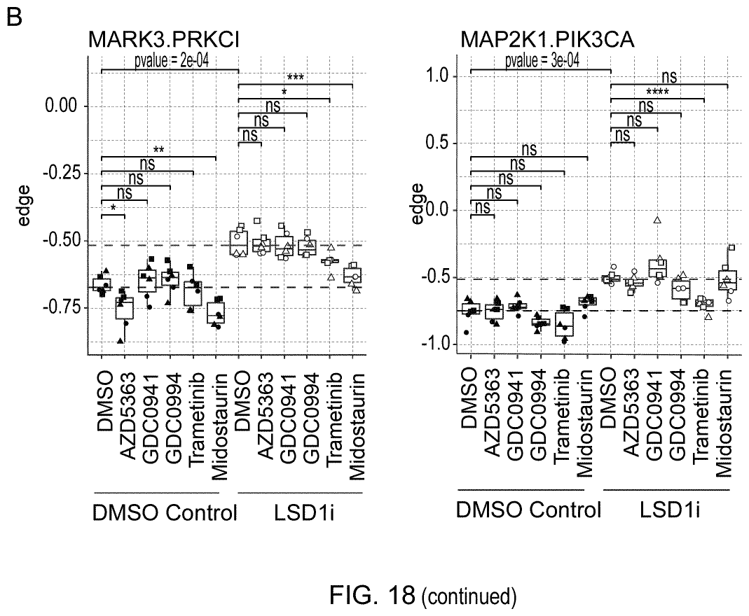

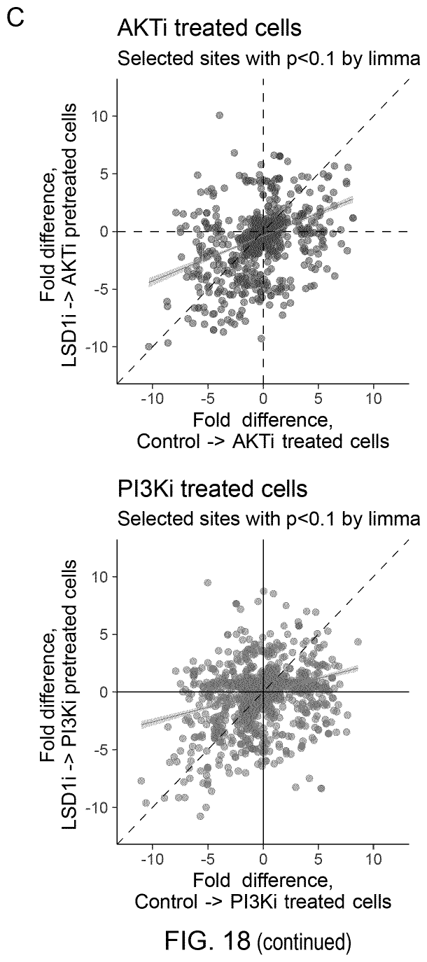

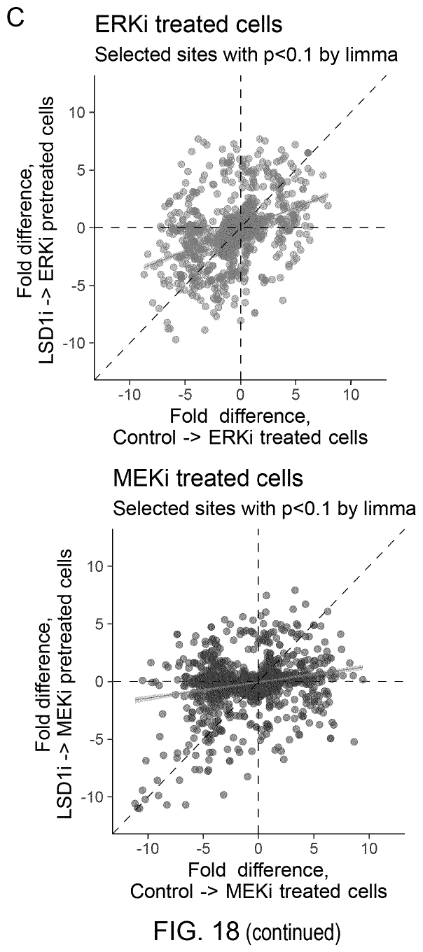

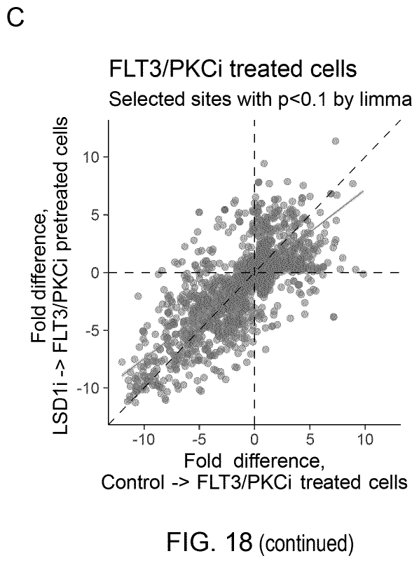

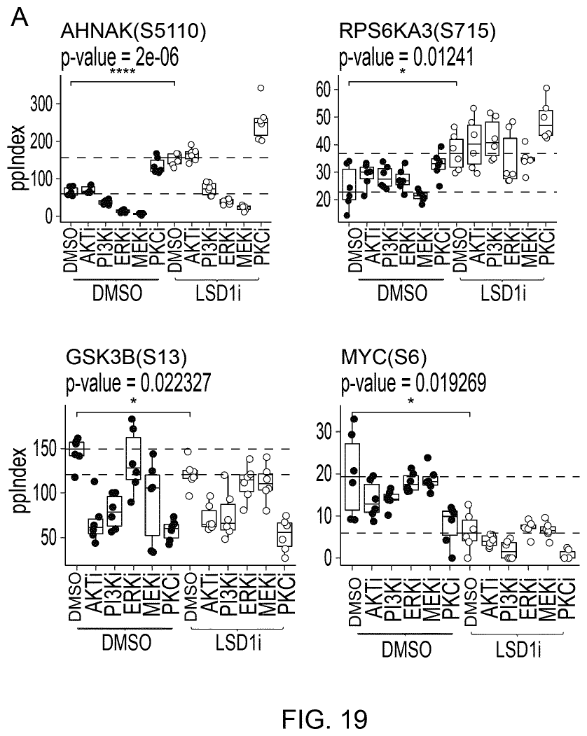

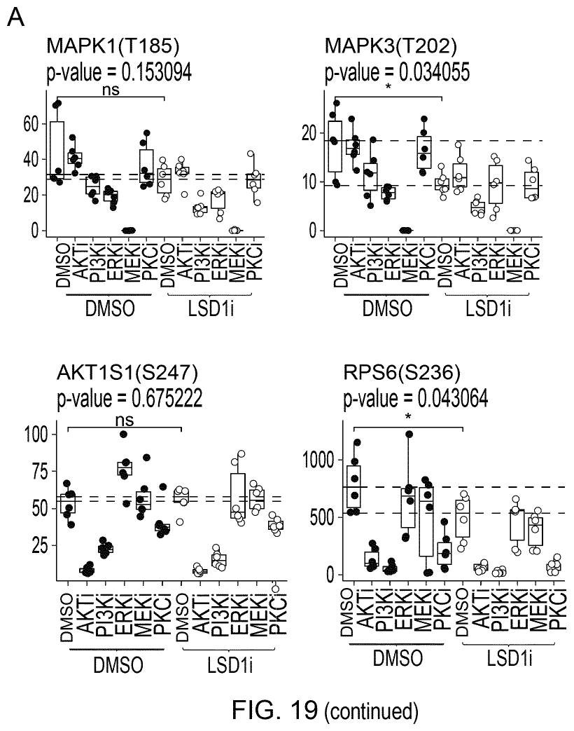

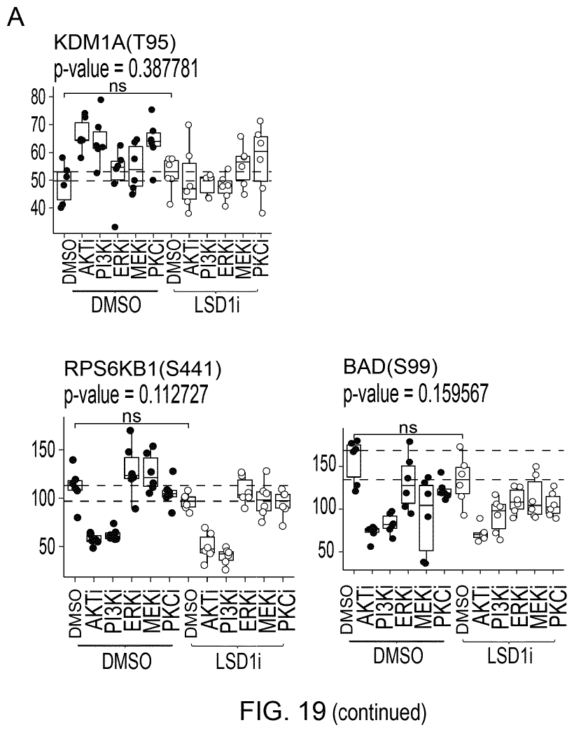

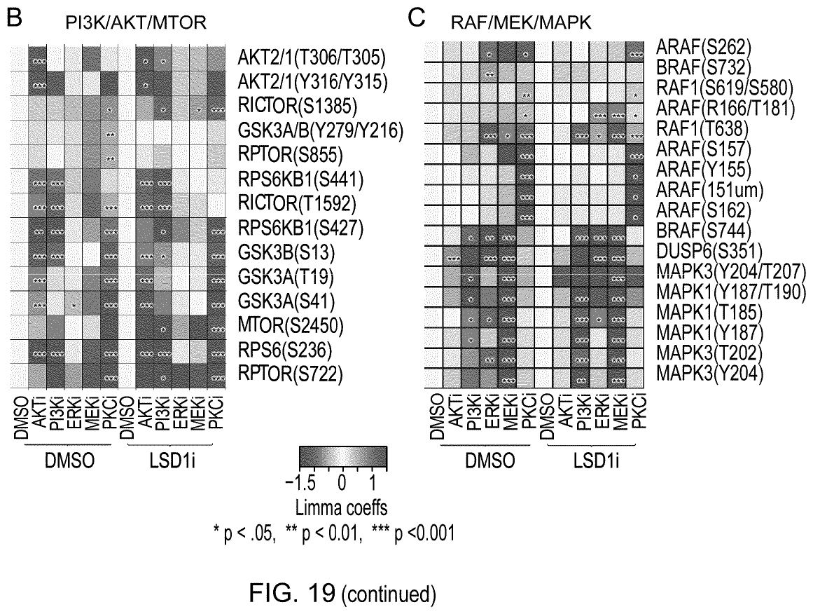

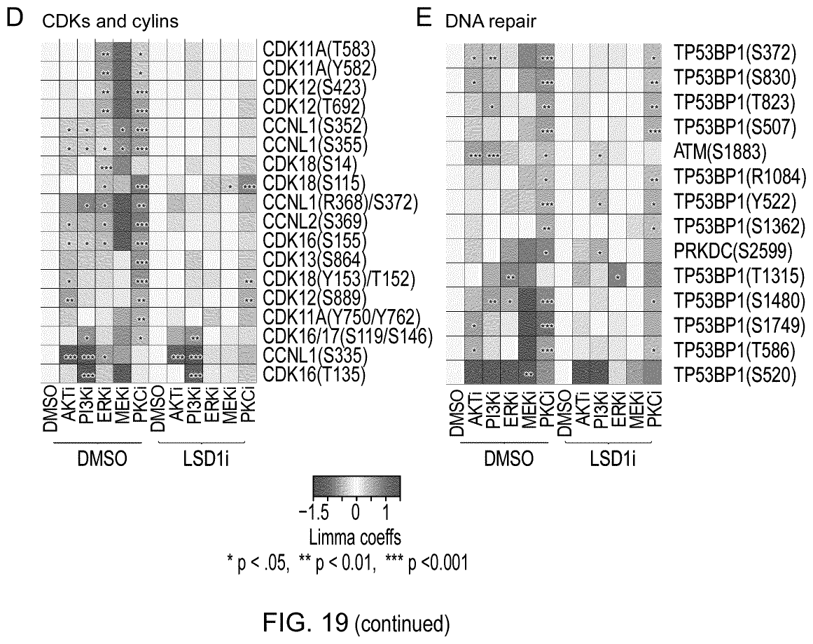

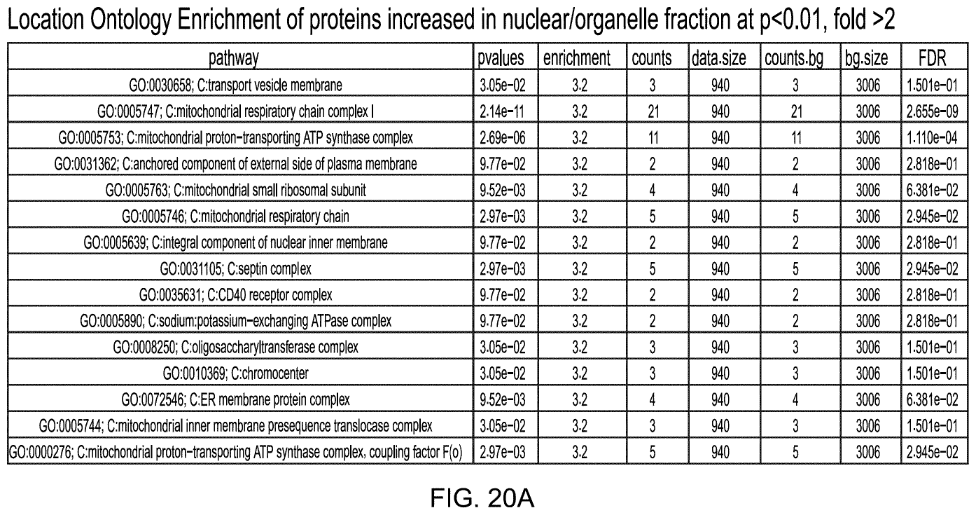

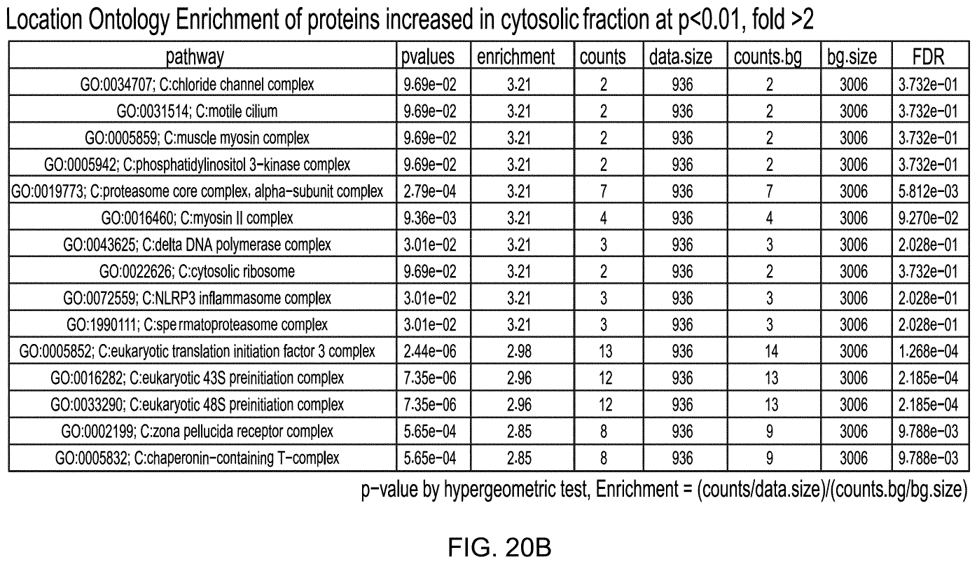

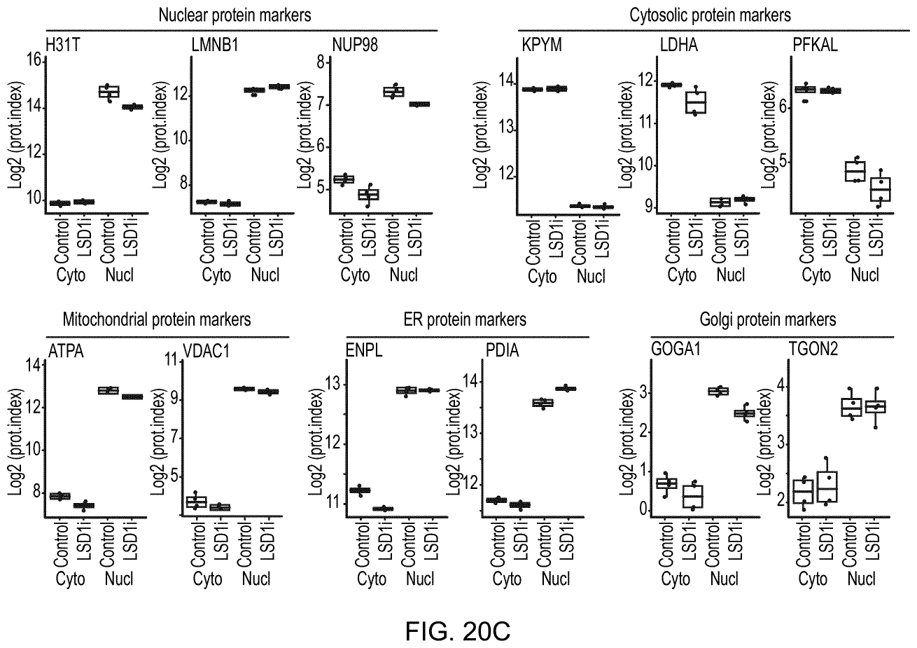

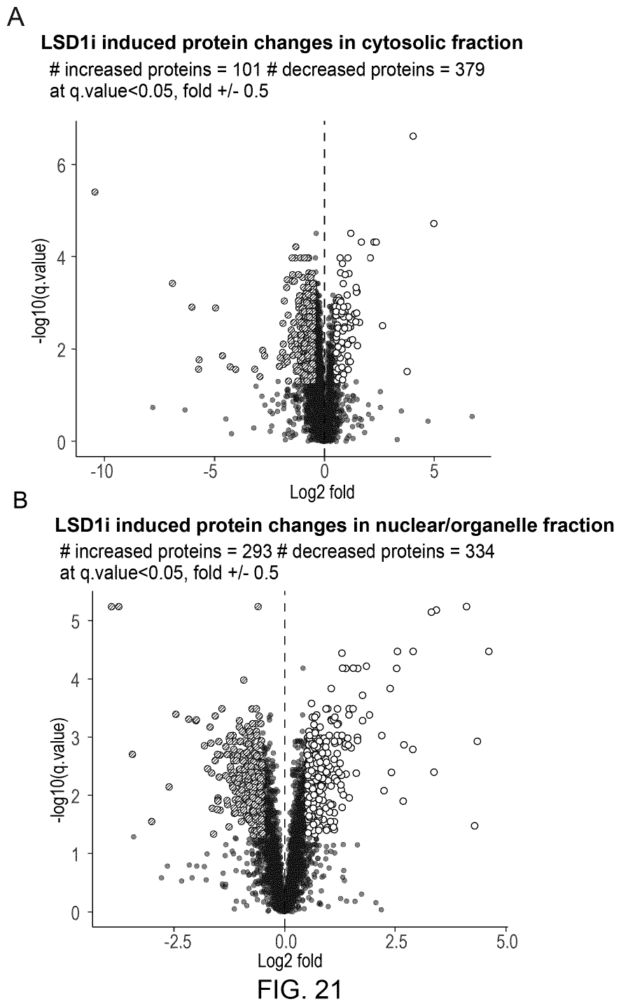

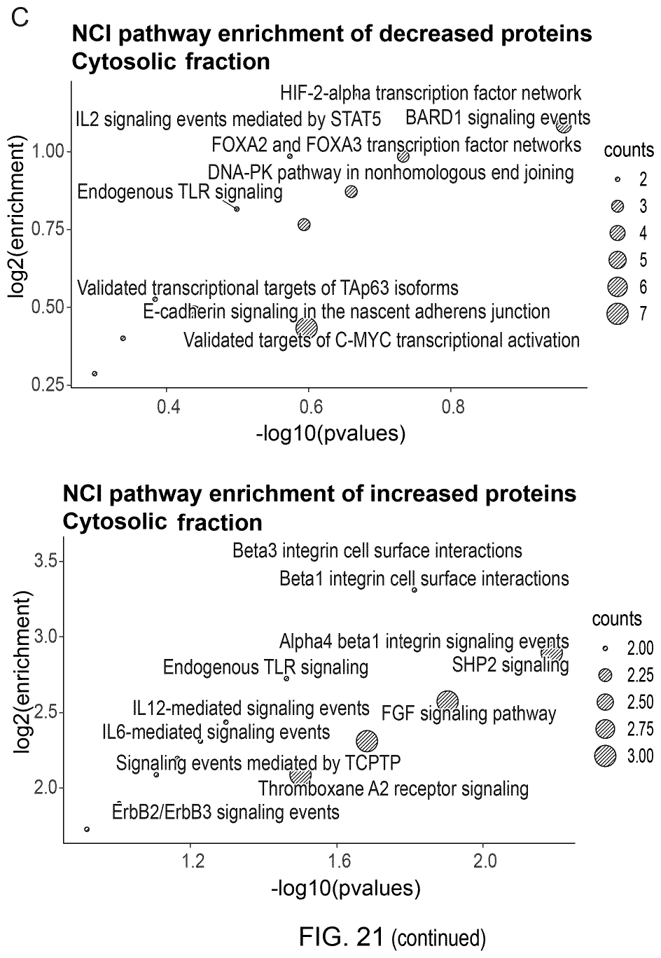

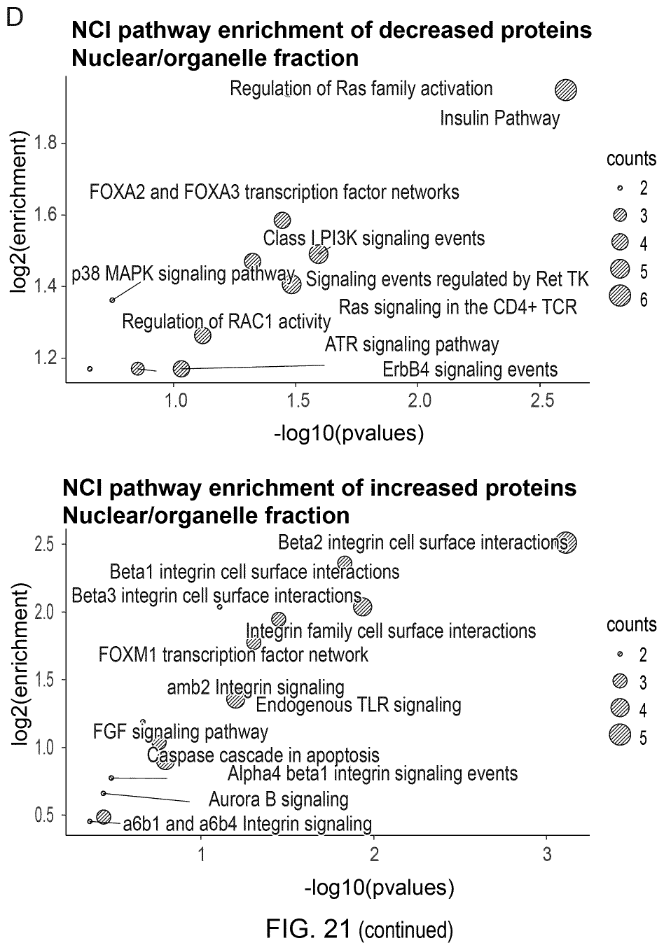

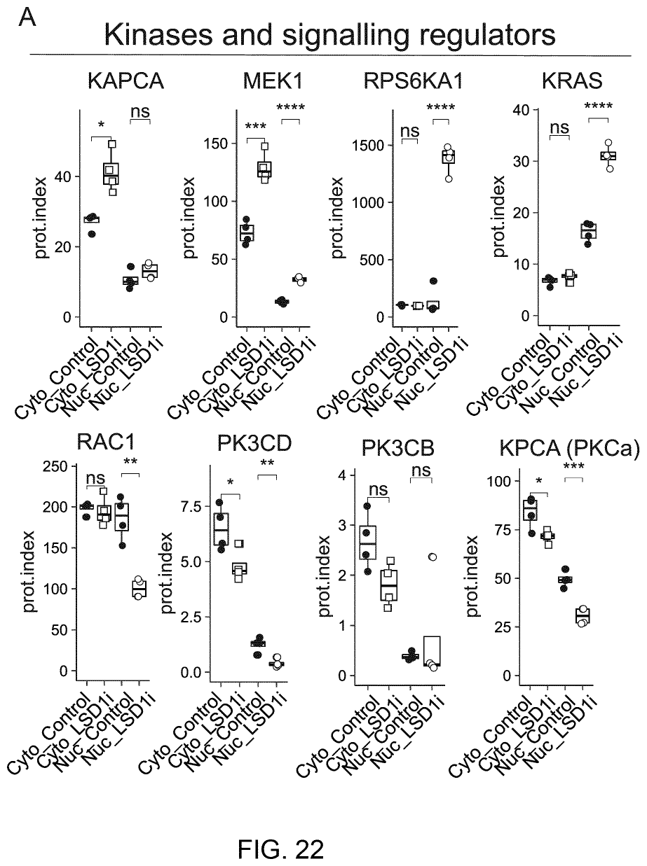

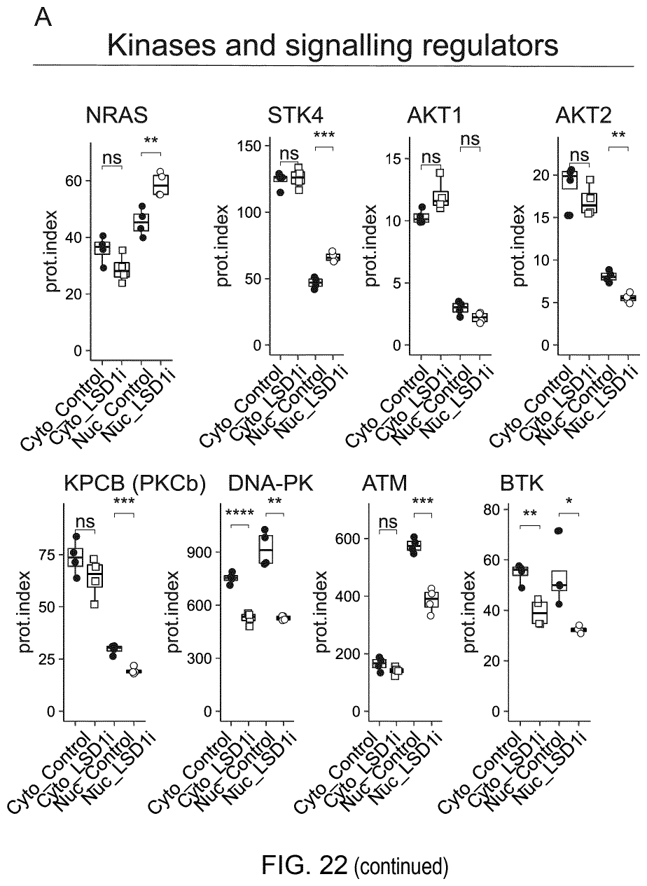

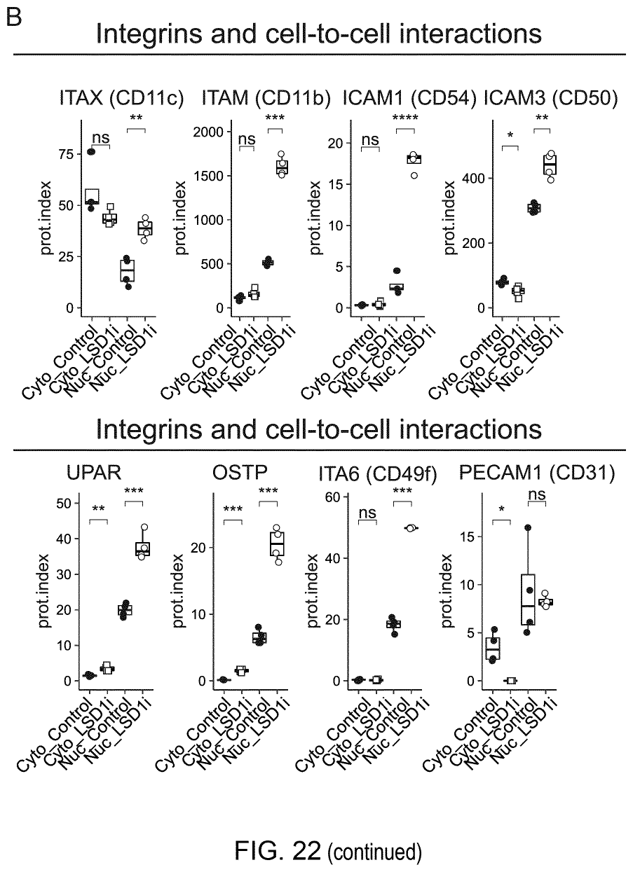

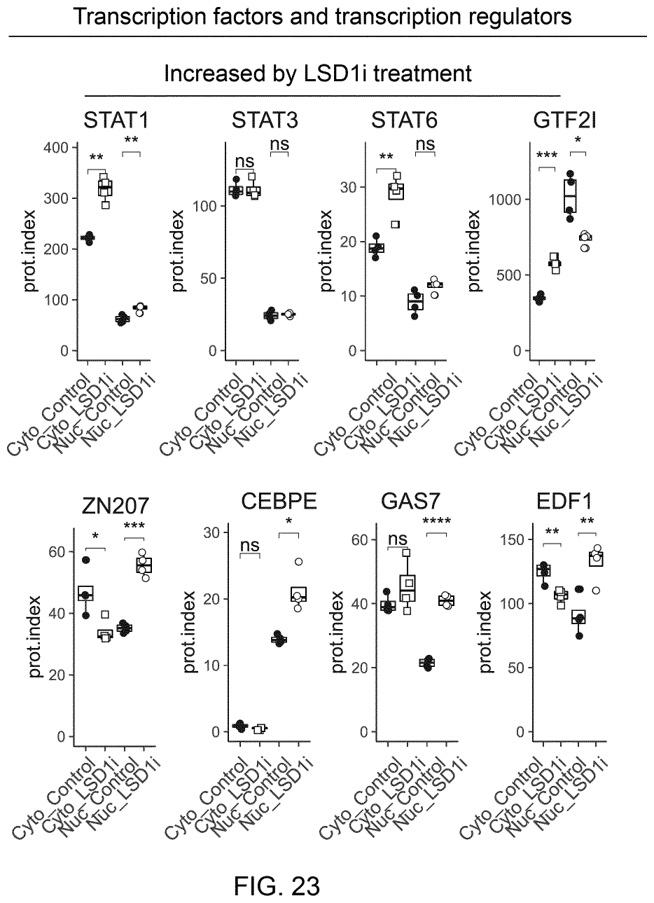

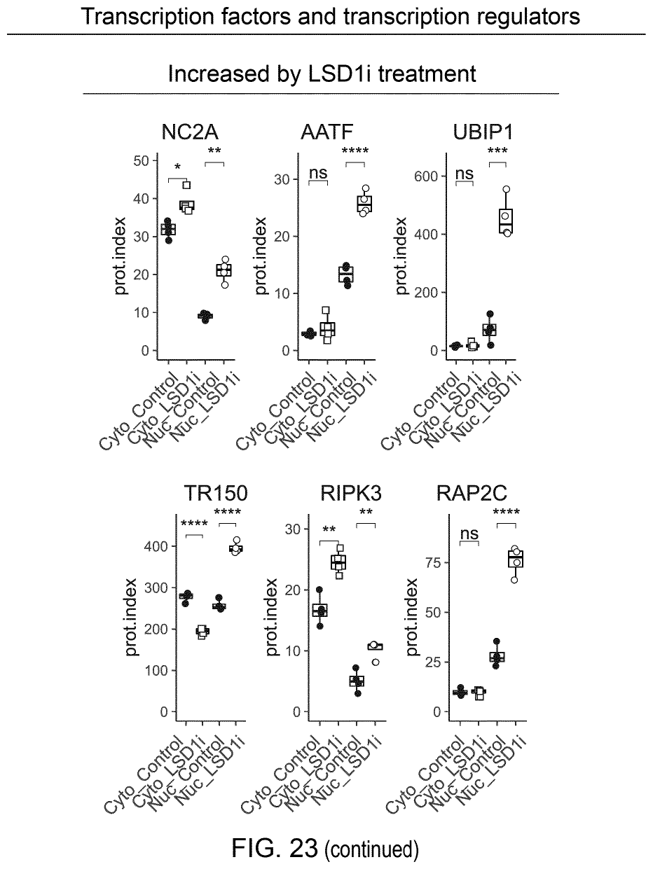

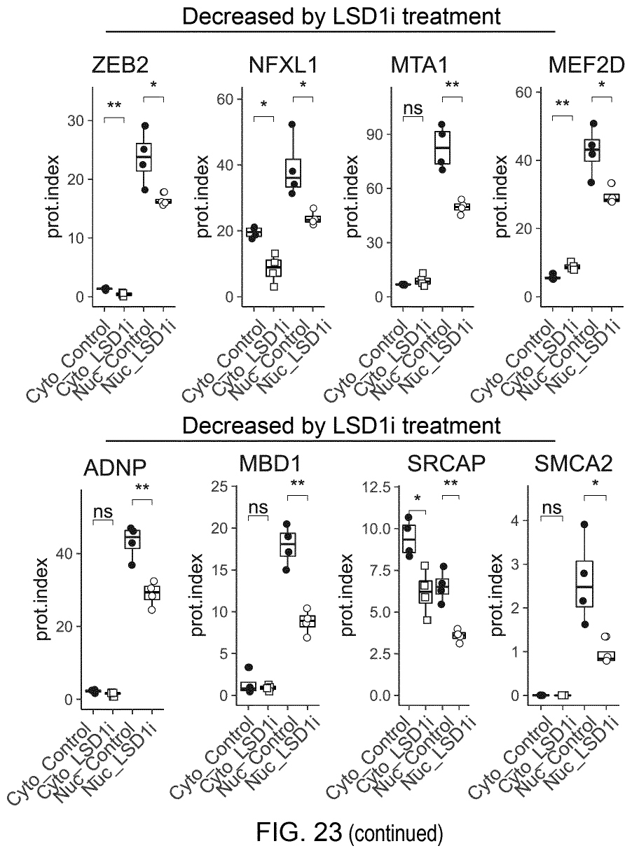

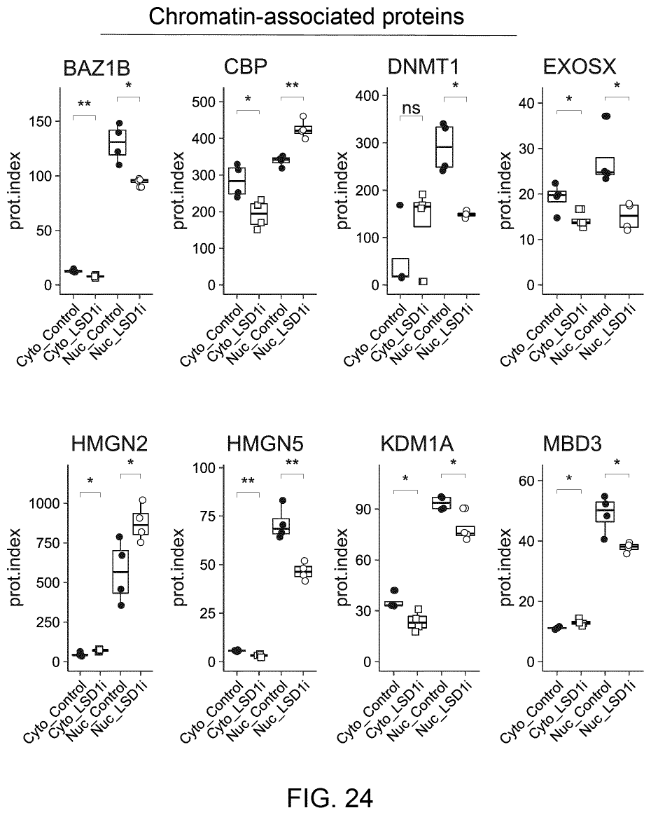

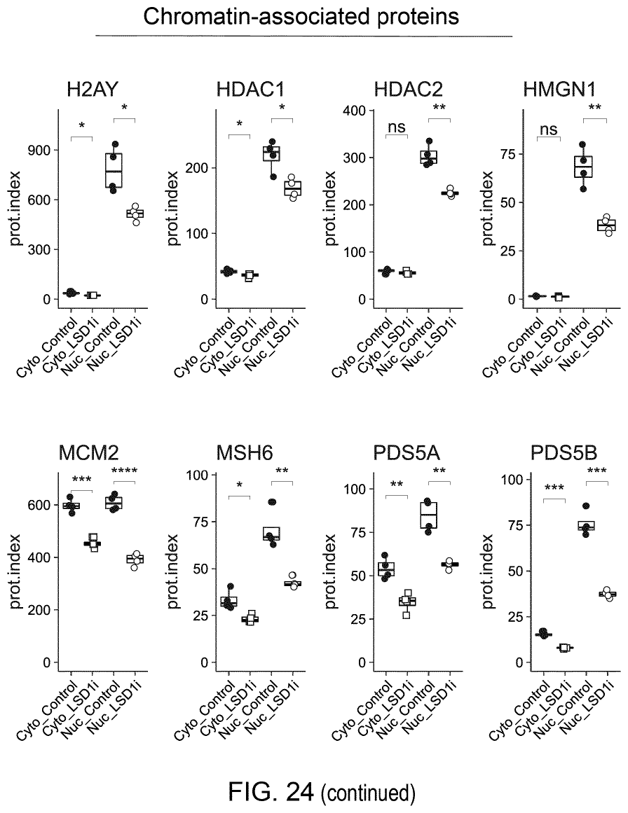

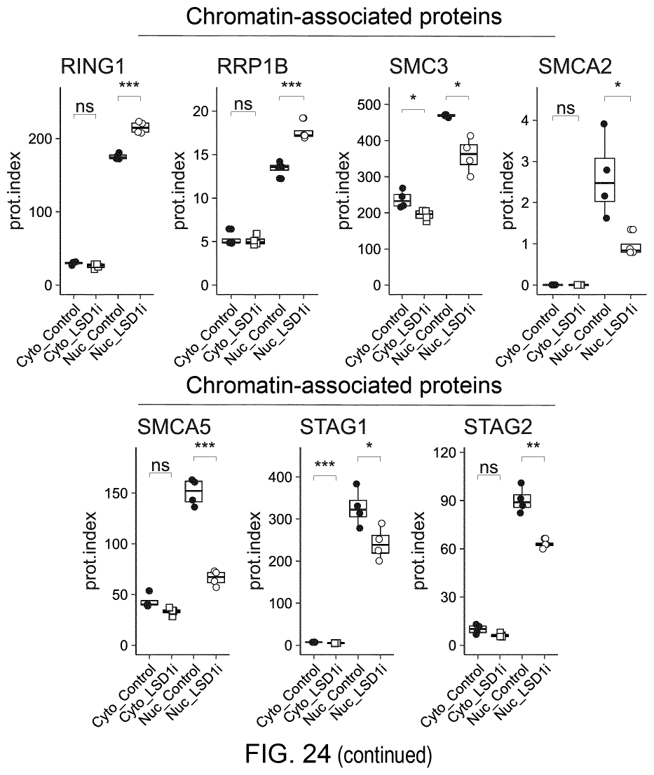

We first compared kinase network circuitries and the extent of haematopoietic differentiation by mining phosphoproteomics and immunophenotype data across 30 primary AML cases. These cases mainly originated from patients of the normal karyotype AML subgroup. Kinase networks were quantified by determining the enrichment of recently reported phosphorylation sites markers of kinase network circuitry (M. Hijazi et al. Nat Biotechnol, (2020)), from which we obtained values of signaling axis activity and of kinase centrality for 1500 signaling axes (i.e. network nodes) and 103 kinases. In general, centrality values correlate with importance of nodes (kinases) in the network. To illustrate the approach that we used to measure kinase circuitry, FIG. 1A shows the quantification of the AKT1.2/MTOR signaling axis across two representative primary AML cases, achieved by measuring phosphorylation sites previously found to be downstream of both AKT (isoforms 1 and 2) and MTOR (M. Hijazi et al. Nat Biotechnol, (2020)). Comparison of this signaling axis with the expression of cluster of differentiation (CD) markers across 30 AML cases showed that AKT1/2-MTOR signaling significantly correlates with the macrophage marker CD11b, while being anti-correlated with the haematopoietic stem/progenitor cell marker CD34 (FIG. 1B).

We systematically applied this approach to 647 signaling network axes and 17 differentiation markers (FIGS. 1, C and D). Relatively mature (CD11b positive) cells showed high activity of several pro-survival signaling pathways including PI3K, AKT1/2, MTOR, CAMKK2, CDK6 and MEK1 (MAP2K1), whereas less differentiated cases (CD34 positive) exhibited increased activity kinases such as TAOK3, MELK, and ERN1 (FIGS. 1, C and D), which are involved in stress response and stem cell self-renewal. Overall, we found signaling axes and individual phosphorylation sites significantly associated with the expression of 17 different markers of cell differentiation, with CD11b, CD64 and CD14 having the largest number of associated network edges (FIG. 1E). Similarly, CD marker expression was also associated with kinase centrality values (FIG. 8A) and with the phosphorylation of epigenetic erasers and writers (FIG. 8B). Our analysis also uncovered specific associations of epigenetic enzyme phosphorylation with kinase network circuitry and kinase node centrality (FIGS. 8, C and D). These results reveal kinase pathways associated to different makers of haematopoietic differentiation in AML.

We then tested whether the variation in network circuitry identified across primary AML cells of distinct differentiation phenotypes had an impact on how cells responded to treatments with kinase inhibitors. We found that CD11b and CD64 expression was associated with sensitivity to trametinib (a MEKi) treatment, while sensitivity to silmitasertib (a CK2i) was greater in cases showing expression of CD117 (c-Kit), CD16 and CD15 (FIG. 8E). Taken together, these data uncover the extent of interdependency between AML cell differentiation, kinase network circuitry and sensitivity to kinase inhibitors.

### Example 2—Agents that Promote Cell Differentiation Prime AML Cells for Kinase Inhibitor Treatment

We found differentiation to be associated with specific patterns of epigenetic marker expression and phosphorylation, kinase activity and sensitivity to kinase inhibitors (FIG. 1, FIG. 8); we therefore asked whether agents that promote cell differentiation would allow us to reshape kinase network circuitry and thus increase the sensitivity of cells to kinase inhibitor treatment. To test this concept and to determine causal effects of differentiation on kinase signaling, we assessed the impact of all-trans-retinoic acid (ATRA, an agent known to promote myeloid cell differentiation) on kinase network re-wiring and sensitivity to MEKi (FIG. 2A). We focused on MEKi because, as noted above, sensitivity to its treatment was strongly associated to the myeloid differentiation markers CD11b and CD64 (FIG. 8E). We found that treatment with ATRA for five days sensitised myeloid P31/Fuj cells to MEKi treatment (FIG. 2B) and decreased the phosphorylation of MAPK1 and PI3K signaling axes, while increasing several axes containing CDKL5 and stress kinases such as STK4 (FIG. 8A). Consistent with these results and with the effects of ATRA in inducing differentiation, ATRA treatment decreased the phosphorylation of the MAPK and PI3K pathway markers ERK1 pY204 and c-MYC pT58/pS62, respectively, while increasing the phosphorylation of CEBPE and LTB4R (leukotriene B₄ receptor 1, a G-protein coupled receptor) in a dose-dependent manner (FIG. 9B). Both CEBPE and LTB4R are associated with mature myeloid cells.

ATRA mediates changes in gene expression by binding to its nuclear receptor, which in turn recruits several epigenetic modifying enzymes, including histone deacetylases, lysine demethylases and polycomb repressive complexes. In addition, the lysine demethylase LSD1/KDM1A regulates ATRA signaling in AML by unknown mechanisms. Thus, since cell differentiation requires changes in chromatin modifications, we hypothesised that chromatin-modifying epigenetic enzymes may also be targeted to remodel kinase pathway activation and thus increase the extent by which AML cells respond to kinase inhibitors.

To test this possibility, we performed a small-scale drug screen (outlined in FIG. 2A) consisting of treatment of cells with representative antagonists of chromatin and DNA modifying enzymes for 5 days (to induce changes in network topology), followed by treatment with MEK, mTOR or PKC inhibitors for 3 days, after which we measured cell viability as a way of testing whether induction of kinase network topology had occurred as a result of epigenetic inhibitor treatment. The Bliss independence model was used to derive coefficients of drug interaction (CDI) values, where CDI <1 indicates significant priming by the epigenetic antagonist (synergistic effect). We found that several epigenetic antagonists sensitised cells to kinase inhibitor treatments (i.e., produced −Log₂ CDI values >0, FIG. 2C). GSK2879552, an inhibitor of the LSD1/KDM1A demethylase, was one of the most potent sensitisers to kinase inhibitor treatment. In a second screen, focusing on LSD1i, we found that LSD1i sensitised 9 out of the 12 AML cell lines tested to midostaurin (which inhibits FLT3, PKC and other kinases), pictilisib (a PI3Ki with selectivity for p110α and p110δ isoforms) or trametinib (a MEK1/2i) (FIG. 2D). In a follow-up experiment, LSD1i sensitised P31/Fuj cells to the effects of MEKi (i.e. suppressing proliferation and inducing apoptosis) in a dose-dependent manner (FIG. 2E). Log₂ CDI values calculated based on the cell number and apoptosis outputs indicated a strongly synergistic effect, with values well below 0 for all concentrations above 10 nM (FIG. 10A), suggesting a strong sensitising effect of the LSD1i pre-treatment to subsequent treatment with MEKi in this cell line model. Previous studies have found that co-treatment with LSD1 and mTOR inhibitors is synergistic in a subset of AML cells. In our hands, a 3-day co-treatment with LSD1i and MEKi did not result in a strong synergism in P31/Fuj cells (FIG. 2F), showing near additive (for proliferation) or mildly synergistic (for apoptosis) CDI values (FIG. 10B). To test whether the stronger synergism observed in the 5+3 days sequential treatment over the 3-day co-treatment was a result of a genuine sensitisation effect of pre-treatment with LSD1i or rather a product of the longer treatment duration, a reverse treatment consisting of 3-day pre-treatment with MEKi followed by a 5-day treatment with LSD1i was tested (FIG. 2F). Contrary to the 5+3-day sequential treatment, this reverse treatment resulted in an additive to strongly antagonistic effect (Log₂ CDI values well above 0) (fig. S3C). Therefore, our data suggest that pre-treatment (rather than co-treatment) with LSD1i enhances the anti-proliferative effects of several kinase inhibitors and that LSD1i primes for MEKi treatment in most cell lines tested. Of note, the sensitisation effect observed was not strictly linked with the choice of LSD1 inhibitor (GSK2879552) in the previous screens, as sequential treatments performed with two alternative LSD1 inhibitors (ORY-1001 and CC-90011) also resulted in synergistic interactions with MEKi in P31/Fuj cells (FIGS. 10, D and E).

We then examined whether, besides cell lines, LSD1i could also sensitise primary AML to kinase inhibitor treatment. We obtained CDI values from 17 AML blasts samples (6 normal, 5 complex and 6 MLL karyotypes) treated for 5 days with LSD1i followed by 3 days with either MEKi, PKC/FLT3i or DMSO control (FIGS. 2, H and I). Primary AML cells were cultured and treated in the presence of a feeder layer of bone marrow stromal cells, which, under control conditions, kept cells viable and proliferating throughout the course of the experiment. Cells growing in this ex-vivo co-culture system have been shown to respond to treatment in a similar way as when implanted in xenograft models. We found that LSD1i primed 8 of the 17 primary AML cases for MEKi treatment (FIG. 2H) while 5 cases (out of the 14 tested) were primed for PKC/FLT3i treatment (FIG. 2I), although this effect was less pronounced compared to sensitisation to MEKi. Also of note, the largest sensitising effect to MEKi treatment could be observed in cases carrying an MLL karyotype, whereas the majority of cases responsive to LSD1i->PKC/FLT3i had a normal karyotype. These results indicate that the LSD1i->MEKi sequential treatment may be particularly effective in about 50% of AML cases.

We next tested whether the LSD1i->MEKi sequential treatment may be affecting healthy human haematopoietic (CD34-positive) cells. We observed an increase in the expression of differentiation markers, such as CD11b and CD14, but no effect on the proportion of CD34-positive cells as a result of treatment with LSD1i (FIG. 11, A to C). LSD1i->MEKi sequential treatment increased apoptosis (FIG. 11D), but this was mostly restricted to the more mature CD34-negative cells (FIG. 12), indicating that the LSD1i->MEKi treatment preserved the normal stem/progenitor cell compartment while inducing apoptosis in differentiated (FIG. 12) and AML cells (FIG. 2).

### Example 3—Multiomic Analysis Uncovers Mutations and Pathways Associated with AML Sensitivity to LSD1i->MEKi Sequential Treatment

Since the extent by which cells respond to LSD1i->MEKi sequential treatment varies across the AML cell lines and patient samples tested (FIGS. 2, D and F), using a multiomic approach (FIG. 3 and FIG. 4), we sought to understand how cells that are sensitive to LSD1i->MEKi differ from those that are resistant. In the AML cell lines tested, the mutational background and morphological classification (FAB) were not on the whole associated with responses to sequential treatment (FIG. 3A and fig. S6). In contrast, we found the expression of proteins and phosphorylation sites belonging to signaling pathways downstream of RTKs to be significantly associated with response to LSD1i->MEKi (FIGS. 3, A and B, FIG. 14). Examples of significant associations for cells sensitive to sequential treatment include higher expression of MAPK1, MAP2K2 (MEK2), KS6A1 (p90 S6K) proteins and of phosphorylation markers associated with MAPK kinase pathway activation (such as EGFR at 5991 and MAP2K2 (MEK2) at T13) (FIGS. 3, A and B, FIG. 14). On the other hand, resistance to sequential treatment was associated with higher levels of expression of MTOR protein and phosphorylation sites markers of MTOR activity, such as T2446 and S2448 (FIG. 3B, FIG. 14). In the primary AML cases tested (FIG. 4A), cells morphologically classified as FAB M5 (which mainly consist of relatively mature myeloid cells) and those carrying an MLL translocation showed a trend towards better response compared to the other subgroups tested (FIG. 15). Similarly, mutational background was associated with response to sequential treatment (FIG. 4B, FIG. 16); in particular, cells carrying a KRAS mutation were found to be significantly more responsive than those wild-type for this gene, whereas cells carrying NRAS or NPM1 mutations were found to be more resistant (FIG. 4B). Based on the proteomics and phosphoproteomics data available for a subset of the AML cases tested (FIG. 4A), strong trends involving components and markers of signaling downstream of RTKs comparable to those observed in cell lines could not be found. Nevertheless, there was a trend towards an association with a better response to treatment in cells expressing higher levels of BRAF and MAP2K3 proteins, both of which act upstream of MEK1/2 (FIG. 4C, FIG. 17). Sensitive cells were also significantly associated with higher expression of markers of PAK1 activity (phosphorylations at T185 and T219), as well as showing a trend towards higher expression of phosphorylation sites at components of the MAPK signaling pathways, such as MAPK3 (ERK1) at T207 and PTPN7 at S359 (FIG. 4C, FIG. 17).

AML is a highly heterogeneous disease; we thus postulated that different and complex combinations of pathway activities could all be contributing to the drug response phenotype. We rationalized that this complexity may be better modelled using tree-based machine learning algorithms designed to model complex and non-linear relationships. We thus used random forest (RF) to integrate our multiomic data into unified models of LSD1i->MEKi drug responses. RF models, constructed by leave-one-out cross-validation, showed low errors (FIGS. 5, A and B), indicating that the drug response data had been successfully modelled. Phosphorylation sites and protein markers showed greater contribution to the models than genetic mutations (FIGS. 5, C and D), suggesting that these sites and proteins may be the greater contributors to responses to sequential treatment and could be used to predict sensitivity to sequential treatment. This was also true of primary AML cells: even though strong patterns were observed with regards to morphological classification and especially mutational background, the top contributors to the model were proteins and phosphorylation sites associated with RTK signaling (FIG. 5D). Interestingly, although KDM1A/LSD1 expression was poorly correlated with response (FIG. 17), it was the third highest contributing marker to the machine learning multiomic model (FIG. 5D), suggesting that LSD1 expression, together with other components of RTK signaling, may be an important determinant of response. Overall, these data indicate that, at the basal untreated state, AML cells susceptible to sequential LSD1i->MEKi treatment activate classical oncogenic pathways downstream of RKTs and RAS, including PI3K/MTOR and MEK/MAPK, while KRAS mutational status and the M5 FAB subgroup were also found to be strong predictors of response in primary AML cells.

### Example 4—LSD1i Induces a PI3K/AKT to MEK/MAPK Signaling Switch and Rewires Kinase Circuitries that could Compensate for MEK Inhibition

To understand how LSD1i modulates kinase signaling so that previously resistant cells become sensitive to MEKi treatment, we analysed kinase networks by phosphoproteomics in P31/Fuj cells treated with LSD1i for 5 days followed by short treatments with inhibitors of AKT1/2, PI3K, ERK1/2, MEK1/2 or PKC/FLT3 (FIG. 6A, FIG. 18A). We found that kinase inhibitors affected fewer phosphorylation sites in cells previously treated with 0.5 μM LSD1i than in cells pre-treated with vehicle (FIG. 6B). LSD1i treatment, by itself, decreased the enrichment of 18 network axes while increasing just 6 of them (at 2.5 z-score threshold, FIG. 6C) and it decreased more kinase activities than it increased (FIG. 6D). Circuitries containing AKT and MTOR were found to decrease, whereas those with PKCI and MEK1 (MAP2K1) increased (FIGS. 6, C and D, FIG. 18B). Comparing the impact of kinase inhibitor treatment on kinase circuitries (FIG. 6E) and on individual phosphorylation sites (FIG. 18C) across cells pre-treated or not with LSD1i produced scatter plots with slopes <1 for all compounds, with smaller slopes seen for PI3Ki, AKTi, MEKi and ERKi comparisons. A slope close to 1 would indicate a similar impact of kinase inhibitors in control and LSD1i pre-treated cells, while a slope <1 or >1 would indicate a higher impact of the kinase inhibitors in the DMSO or LSD1i treated cells, respectively. These results indicate that LSD1i pre-treatment blunted the impact that inhibitors of PI3K, AKT, MEK and ERK have in reducing kinase signaling, and indicate that LSD1i treatment reduces the activity of several pro-survival kinase signaling pathways.

Analysis of individual phosphorylation sites confirmed that LSD1i decreased the phosphorylation of some sites known to be downstream of the PI3K/AKT pathway (FIG. 19A). As an example, LSD1i reduced c-Myc phosphorylation at S62, and kinase inhibitor treatments confirmed that this site is downstream of PI3K and AKT (but not MEK, ERK or PKC) in this cellular model (FIG. 6F). The PI3K/AKT pathway markers GSK3B pS13 and RPS6 p236, and the MAPK pathway marker MAPK3 (ERK1) pT202 also decreased as a result of LSD1i treatment, while AHNAK pS5110 (a site downstream of both PI3K and MEK/ERK pathways) increased (FIG. 19A). Interestingly, we found that kinase inhibitor treatments induced an increase in the phosphorylation of LSD1/KDM1A at T95 and S93 in control cells but not in cells pre-treated with LSD1i (FIG. 6G, FIG. 19A), suggesting that LSD1i suppressed a negative feedback on its LSD1/KDM1A target. This finding prompted us to assess the effects of LSD1i in suppressing feedback loops in a more systematic manner. We found that while PI3K, AKT, MEK and ERK inhibitors decreased the expected signaling pathways in both LSD1i pre-treated and control cells (FIGS. 19, B and C), LSD1i treatment blunted the effect of these compounds in increasing the phosphorylation of epigenetic regulators and histones (FIG. 6H), as well as CDKs, cyclins and proteins involved in DNA repair (FIGS. 19, D and E). Thus, LSD1i induces a PI3K to MEK signaling switch and reduces the ability of cells to modulate feedback loops revealed by kinase inhibitor treatments.

To investigate the source of the observed signaling switch induced by LSD1i, we compared the nuclear/organelle and cytosolic proteomes of cells treated with LSD1i versus control (FIG. 7A, FIG. 209). These experiments quantified >5,200 proteins in these fractions, and allowed us to assess the impact of LSD1i treatment in protein expression and subcellular localization. We found that LSD1i induced profound changes in 101 and 293 proteins increased in cytosolic and nuclear/organelle fractions, respectively, with 379 and 334 proteins decreasing as a result of LSD1i treatment (FIGS. 21, A and B). Pathway analysis of the proteomics data showed that LSD1i induced a decrease in proteins involved in the PI3K, insulin and c-MYC signaling pathways (consistent with the phosphoproteomics data, FIG. 6) and an increase in α and β-integrin signaling proteins (FIGS. 21, C and D, FIG. 22 and FIG. 23). Of note, LSD1i induced an increase in the expression of MAPK pathway enzymes including NRAS, KRAS, MEK1 and P90-S6K (RPS6KA1 isoform) and a decrease in the expression of PI3K p110δ catalytic isoform, AKT2, and RAC1 (FIG. 7B, FIG. 22A), among other kinases, kinase regulators, integrins, transcription factors and chromatin regulators (FIG. 22, FIG. 23 and FIG. 24). Together, the phosphoproteomics, proteomics and pathway analysis data indicate that LSD1i dampens the overall activity of pro-survival pathways, and induces a signaling switch in which a decrease in PI3K-AKT signaling is accompanied by an increase in RAS-MEK-MAPK pathway activation.

### Example 5—Materials and Methods for Examples 1 to 4

### Cell Culture and Treatment of AML Cell Lines

CMK (ref: ACC 392), HEL (ref: ACC 11), HL-60 (ref: ACC 3), Kasumi-1 (ref: ACC 220), KG-1 (ref: ACC 14), ML-2 (ref: ACC 15), MOLM13 (ref: ACC 554), NB-4 (ref: ACC 207) and NOMO-1 (ref: ACC 542) were obtained from the DSMZ collection. MV4-11 (ref: CRL 9591), and HS-5 (ref: CRL 11882) were obtained from the ATCC collection. P31/Fuj (ref: JCRB0091) was obtained from the JCRB collection. AML cells were maintained in RPMI-1640 medium supplemented with 10% (v/v) heat inactivated FBS and 1% (100 U/mL) Penicillin/Streptomycin (P/S) (RPMI/FBS medium), while MS-5 cells were maintained in IMDM medium supplemented with 10% heat inactivated FBS and 1% P/S. All cell lines were maintained at 37° C. and 5% CO₂ in a humidified environment.

For the proliferation/viability assays, cells were seeded in T75 flasks (0.5×10⁶ cells/mL of RPMI/FBS medium) and exposed for 5 days to either vehicle or epigenetic modifier. Unless otherwise specified, the final concentration for GSK2879552 (LSD1i), CDI-1205 (EZH2i), pinometostat (DOT1Li) and ATRA was 1 μM while for decitabine (DNMTAi) and vorinostat (HDACi) was 100 nM; all epigenetic inhibitors were diluted in DMSO, while ATRA was diluted in Et-OH. Vehicle concentration was kept at 0.1%. Following the initial 5-day treatment, cells were counted, centrifuged, resuspended in fresh RPMI/FBS medium, then seeded in 6-well plates (0.25×10⁶ cells/mL) and exposed to either DMSO or kinase inhibitor for 3 days. The kinase inhibitors midostaurin (FLT3/PKCi), trametinib (MEKi), pictilisib (PI3Ki) and torin-1 (mTORi) were diluted in DMSO and used at a final concentration of 1 μM, unless otherwise specified. The final concentration of DMSO was kept at 0.1%. Finally, cells were transferred to 96-well plates (100 μL per well in 3 technical replicates per condition) and cell viability and proliferation were assessed using Guava and ApoTox-Glo Triplex analyses, as indicated below.

To study ATRA effect over the phosphoproteome, P31/Fuj cells (10×10⁶ cells at 0.5×10⁶ cell/mL) were seeded in T75 flask and treated with either Et-OH, 1 μM ATRA or 10 μM ATRA for 5 days. To study the effect on the phosphoproteome of LSD1 inhibitor exposure followed by kinase inhibitor treatment, P31/Fuj cells (20×10⁶ cells at 0.5×10⁶ cell/mL) were seeded in T75 flask and treated with either DMSO or 0.5 μM GSK2879552 (LSD1i) for 5 days. Following that, cells were counted, centrifuged, resuspended in fresh RPMI/FBS medium, then seeded in T25 (14×10⁶ cells in 10 mL) and treated with DMSO or 1 μM of midostaurin (FLT3/PKCi), trametinib (MEKi), pictilisib (PI3Ki), AZD5363 (AKTi) or GDC-0994 (ERKi) for 1 h. For both experiments, cells were collected by centrifugation (1500 rpm for 5 min at 5° C.), then cell pellets were washed twice with ice cold PBS supplemented with phosphatase inhibitors (1 mM Na₃VO₄, 1 mM NaF) and stored at −80° C. Pellets were processed for phosphoproteomics analysis as indicated below.

For the cell fractionation, P31/Fuj cells (5×10⁶ cells at 0.5×10⁶ cell/mL) were seeded in T75 flask and treated with either DMSO or 0.5 μM GSK2879552 (LSD1i) for 5 days. After that, cells were collected by centrifugation (1500 rpm for 5 min at 5° C.), then cell pellets were washed twice with ice cold PBS supplemented with phosphatase inhibitors (1 mM Na₃VO₄, 1 mM NaF) and stored at −80° C. Isolation of nuclear/organelle and cytosolic fractions for proteomic analysis was carried out as described below.

### Culture and Treatment of Primary Samples

Umbilical cord bloods were purchased from Anthony Nolan. Mononuclear cells (MNCs) were isolated by Ficoll prior red blood cells (RBC) lysis buffer (BioLegend, catalog #420301). Cells were then stained with CD34 immunomagnetic positive selection kit (EasySep™ Human CD34 Positive Selection Kit II, StemCell technologies, catalog #17856, according to manufacturer instructions). Purity of CD34⁺ cells isolated from MNCs was assessed by flow cytometry prior to long term storage in liquid nitrogen. For AML samples, patients gave informed consent for the storage and use of their blood cells for research purposes. Experiments were performed in accordance with the Local Research Ethics Committee, as previously described. Mononuclear cells from peripheral blood or bone marrow biopsies were isolated in the BCI tissue bank facility and stored in liquid nitrogen.

Cell vials were thawed in 37° C. water bath and contents transferred to 15 mL Falcon tubes containing 4 mL of pre-warmed FBS with 40 ng/mL DNase (Sigma Aldrich D4513-1VL). Suspension was centrifuged at 300×g for 8 minutes, then supernatant was removed and cells resuspended in MyeloCult H5100 medium (Stemcell Technologies #05150) containing 1% P/S and supplemented with human IL-3 (Peprotech #200-03), human G-CSF (Peprotech #300-23) and human TPO (#300-18), each to a final concentration of 20 ng/mL.

For the establishment of the feeder layer, MS-5 cells were plated onto 6-well plates (for the co-culture with CD34+ cord blood cells) or 96-well plates (for the co-culture with primary AML cells) at a density of 5-10,000 cells/cm² (48,000 cells for each well of a 6-well plate, or 3,200 cells for each well of a 96-well plate) with IMDM medium. Cells were then incubated at 37° C. for 48 hours, or until a confluency of approximately 70% was reached, after which they were irradiated with an X-ray source at 680 cGy. Following irradiation, the growth medium was removed and replaced with complete H5100 medium, and cell were incubated at 37° C. for a further 24 hours.

Following recovery, cord blood-derived CD34+ cells or primary AML cells were added to the plates containing the established MS-5 feeder layers. CD34+ cells were added at a density of 20,000 cells/well of a 6-well plate, whilst primary AML cells were added at 8,300 cells/well of a 96-well plate. Cells were then left to incubate at 37° C. and to recover for 4 days, after which treatments were started. Cells were first pre-treated with either DMSO or 1 μM GSK2879552 (LSD1i) for 5 days, after which they were treated with either DMSO or 1 μM trametinib (MEKi) (for CD34+ cells) or additionally 1 μM midostaurin (PKC/FLT3i) (for primary AML cells) for 3 days. Following treatment, CD34+ cells were harvested by collecting culture medium in FACS tubes. Adherent cells were detached by briefly incubating with trypsin-EDTA, then also added to the respective FACS tubes. Cells were centrifuged at 300×g for 8 minutes, the supernatant was removed and cells were resuspended in 1 mL PBS+2% FBS, then processed for FACS analysis as described below. For primary AML, cells were resuspended and transferred to separate 96-well plates, after which they were processed for Guava ViaCount analysis, as described below.

### Cell Proliferation, Viability and Apoptosis Assays

For Guava analysis, cells were stained with Guava ViaCount reagent (Luminex #4000-0040) in 96-well plates, by mixing 150 μL of cell suspension with 75 μL of reagent, and left to incubate for a minimum of 10 minutes in the dark, after which they were processed through a Guava easyCyte flow cytometer (Luminex #0500-5005). Flow cytometry data were analysed using CytoSoft (v2.5.7). Viability and cell number values were expressed relative to vehicle control.

For ApoTox-Glo analysis, cell suspensions were transferred to opaque 96-well plates (100 μL per well) and viability and apoptosis measured using ApoTox-Glo Triplex Assay (Promega #G6320). 20 μL of Viability/Cytotoxicity reagent (containing GF-AFC and bis-AAF-R110 substrates) were added to each well, then incubated at 37° C. for 1 hour. Live cell and cytotoxicity outputs were measured on FLUOstar Omega microplate reader (BMG Labtech). To obtain the live cell output, fluorescence was measured at 355_{EX}/520_{EM} (wavelengths in nm); for the cytotoxicity output, fluorescence was measured at 485_{EX}/520_{EM}. Following this, 100 μL of Caspase-Glo 3/7 reagent were added to each well and incubated at 25° C. for 1 hour. Luminescence was then measured on FLUOstar Omega microplate reader (BMG Labtech). The fluorescence and luminescence signals thus obtained were corrected to the respective background controls (culture medium with no cells). Relative cell number values were obtained by normalising live cell outputs to vehicle control. Apoptosis values were obtained by dividing luminescence outputs to the respective live cell outputs, then normalising to vehicle control.

### FACS Analysis

After the indicated treatments, cells were washed in 1 mL PBS+2% FBS and centrifuged at 300×g for 8 minutes, then resuspended in 100 μL antibody staining solution and incubated at 4° C. for 20 min. Antibody staining solution was prepared by mixing Annexin V binding buffer (Biolegend #422201) with the following antibodies/dyes: PE anti-mouse SCA1 (from EasySep mouse SCA1 positive selection kit, Stemcell Technologies #18756; at 2 μL/mL); APC Annexin V (Biolegend #640919; at 36 μL/mL); PerCP/Cyanine5.5 anti-human CD34 (Biolegend #343611; at 18 μL/mL); PE/Cyanine7 anti-human CD45 (Biolegend #368532; at 18 μL/mL); Brilliant Violet 605 anti-human CD11b (Biolegend #301331; at 36 μL/mL); APC/Cyanine7 anti-human CD14 (Biolegend #367107; at 18 μL/mL); Alexa Fluor 488 anti-human CD86 (Biolegend #305413; at 18 μL/mL). Following incubation, cells were washed with 1 mL Annexin V binding buffer and centrifuged at 300×g for 8 minutes. The supernatant was then removed and cells were resuspended in 300 μL Annexin V binding buffer containing 100 ng/mL DAPI. FACS analysis was performed on BD LSRFortessa (BD Biosciences) and data were analysed using FlowJo (v10).

### Phosphoproteomics

Phosphoproteomics analysis was carried out as described previously M. Hijazi et al. Nat Biotechnol, (2020). In brief, cell pellets were lysed in 300 μL of urea buffer (8 M urea in 20 mM HEPES, pH 8.0, supplemented with 1 mM Na₃VO₄, 1 mM NaF, 1 mM Na₄P₂O₇ and 1 mM P-glycerophosphate). Lysates were further homogenized by sonication (30 cycles of 30 s on 30 s off; Diagenode Bioruptor® Plus) and insoluble material was removed by centrifugation. Protein was quantified using BCA (Thermo Fisher Scientific). Then, 500 μg of protein were subjected to cysteine alkylation using sequential incubation with 10 mM dithiothreitol (DDT) and 16.6 mM iodoacetamide (IAM) for 1 h and 30 min, respectively, at 25° C. with agitation. Trypsin beads (50% slurry of TLCK-trypsin; Thermo Fisher Scientific; Cat. #20230) were equilibrated with 3 washes with 20 mM HEPES (pH 8.0), the urea concentration in the protein suspensions was reduced to 2 M by the addition of 20 mM HEPES (pH 8.0), 100 μL of equilibrated trypsin beads were added and the samples were incubated overnight at 37° C. Trypsin beads were removed by centrifugation (2000×g at 5° C. for 5 min) and the resulting peptide solutions were desalted through Oasis HLB cartridges (Waters) following the manufacturer's indications. Briefly, cartridges were set in a vacuum manifold device and the pressure was adjusted to 5 mmHg. Then, cartridges were conditioned with 1 mL acetonitrile (ACN) and equilibrated with 2.5 mL of wash solution (0.1% trifluoroacetic acid (TFA), 2% ACN). Peptides were loaded in the cartridges and washed with 1 mL of wash solution. Finally, peptides were eluted with 500 μL of glycolic acid buffer 1 (1 M glycolic acid, 5% TFA, 50% ACN). Enrichment of phosphorylated peptides was performed with TiO₂ Beads (GL Sciences). The eluents were normalized to 1 mL with glycolic acid buffer 2 (1 M glycolic acid, 5% TFA, 80% ACN) and incubated with 50 μl of TiO₂ buffer (50% slurry in 1% TFA) for 5 min at room temperature. TiO₂ beads were packed by centrifugation into empty spin columns (Glygen Corporation; Cat. TT2EMT) previously washed with ACN. TiO₂ beads were sequentially washed by centrifugation (1500×g for 3 min) with 100 μL of glycolic acid buffer 2, ammonium acetate solution (100 mM ammonium acetate in 25% ACN) and twice with neutral solution (10% ACN). For phosphopeptide elution, spin tips were transferred to fresh tubes, 50 μL of elution solution (5% NH₄OH, 7.5% ACN) were added and tips were centrifuged at 1500×g for 3 min. The elution step was repeated a total of 4 times. Finally, samples were snap frozen, dried in a SpeedVac and phosphopeptide pellets were stored at −80° C.

### Subcellular Proteomics

The isolation of cytosolic and nuclear/organelle proteins was performed as follows. Cells (10×10⁶ cells per replicate) were resuspended in 1 mL hypotonic lysis buffer (containing 10 mM Tris-HCl pH 8.0, 1 mM KCl, 1.5 mM MgCl₂, 1 mM DTT, 1 mM Na₃VO₄ and 1 mM NaF) and incubated at 4° C. for 30 minutes on a rotator. Nuclei/organelles were then pelleted (10,000×g, 10 min, 4° C.), after which the supernatant (cytosolic fraction) was transferred to fresh tubes. The nuclear/organelle fraction was further processed for proteomic analysis by adding 8M urea buffer as indicated above.

Proteins in the supernatant were isolated by methanol/chloroform precipitation as follows. Methanol was added to the protein solution (at 4×initial volume), followed by chloroform (1×initial volume) and ddH₂O (3×initial volume), vortexing after each addition. Samples were centrifuged for 2 min, 12,000×g, 4° C., and the top aqueous layer was removed. Additional methanol was added (4×initial volume) and samples vortexed. Samples were centrifuged for 3 min, 12,000×g, 4° C., and the supernatant removed. Protein pellets were dried briefly at RT, then resuspended in 8M urea buffer. Homogenisation, cysteine alkylation and tryptic digestion of the nuclear and cytosolic fractions were carried out as described above. Desalting was performed through C18 spin columns (Glygen Corporation, Cat. TT2C18) following the manufacturer's indications. Briefly, columns were equilibrated twice with 200 μL 70% ACN, and washed twice with 200 μL 1% ACN. Peptide samples were loaded onto the columns, then washed twice with 200 μL 1% ACN, with spinning. Peptides were eluted four times with 50 μL 70% ACN, then samples were snap frozen, dried in a SpeedVac and peptide pellets were stored at −80° C. Columns were spun for 2 min at 1,500×g, 4° C. each time they were loaded.

### Mass Spectrometry

Mass spectrometry for identification and quantification of proteins and phosphopeptides was carried out by LC-MS/MS as described before. Briefly, peptide pellets were resuspended in 9 μL (for phosphoproteomics) or 50 μL (for proteomics) of reconstitution buffer (20 fmol/μL enolase in 3% ACN, 0.1% TFA) and 5 μL were loaded onto an LC-MS/MS system consisting of a Dionex UltiMate 3000 RSLC coupled to an Q Exactive™ Plus Orbitrap Mass Spectrometer (Thermo Fisher Scientific) through an EASY-Spray source (Cat. ES081, Thermo Fisher Scientific). Mobile phases for the chromatographic separation of the peptides consisted in Solvent A (3% ACN: 0.1% FA) and Solvent B (99.9% ACN; 0.1% FA). Peptides were loaded in a p-pre-column (Acclaim™ PepMap™ 100 C18 LC; Cat 160454, Thermo Fisher Scientific) and separated in an analytical column (Acclaim™ PepMap™ 100 C18 LC; Cat. 164569, Thermo Fisher Scientific) using a gradient running from 3% to 23% B over 60 min (for phosphoproteomics) or 120 min (for proteomics). The UPLC system delivered a flow of 2 μL/min (loading) and 300 nL/min (gradient elution). The Q Exactive Plus operated a duty cycle of 2.1s. Thus, it acquired full scan survey spectra (m/z 375-1500) with a 70,000 FWHM resolution followed by data-dependent acquisition in which the 15 most intense ions were selected for HCD (higher energy collisional dissociation) and MS/MS scanning (200-2000 m/z) with a resolution of 17,500 FWHM. A dynamic exclusion period of 30s was enabled with a m/z window of ±10 ppm.

### Proteomics Bioinformatics

Proteins and phosphorylation sites were identified from mass spectrometry as described before. Peptide identification from MS data was automated using Mascot Daemon 2.5.0 workflow in which Mascot Distiller v2.5.1.0 generated peak list files (MGFs) from RAW data and the Mascot search engine (v2.5) matched the MS/MS data stored in the MGF files to peptides using the SwissProt Database (SwissProt_2012Oct.fasta for proteomics or uniprot_sprot_2014_08.fasta for phosphoproteomics). Searches had a FDR of ˜1% and allowed 2 trypsin missed cleavages, mass tolerance of ±10 ppm for the MS scans and ±25 mmu for the MS/MS scans, carbamidomethyl Cys as a fixed modification and PyroGlu on N-terminal Gln, oxidation of Met and phosphorylation on Ser, Thr, and Tyr as variable modifications (phosphorylation was only included for searches performed using phosphoproteomics data).

Identified peptides were quantified using Pescal software in a label-free procedure based on extracted ion chromatograms (XICs). Thus, the software constructed XICs for all the peptides identified across all samples with mass and retention time windows of ±7 ppm and ±2 min, respectively and calculated the area under the peak. Individual peptide intensity values in each sample were normalized to the sum of the intensity values of all the peptides quantified in that sample. Data points not quantified for a particular peptide were given a peptide intensity value equal to the minimum intensity value quantified for that particular peptide across all samples divided by 10. For the phosphoproteomics experiment with LSD1 and kinase inhibitors, values of 2 technical replicates per sample were averaged. For the proteomics experiment, protein intensity values were calculated by adding the individual normalized intensities of all the peptides comprised in a protein. Only proteins with at least 2 unique quantified peptides were considered. Protein score values were expressed as the maximum Mascot protein score value obtained across samples.

### Computational Biology and Statistics

Unless otherwise stated, statistical analyses were carried out in the R package (version 3.6.1, 2017 #1201}) within the RStudio (v1.1.463) environment. Where indicated in the text, Spearman, Pearson, Wilcoxon, Kolmogorov and t-tests were performed using the respective base R functions. Adjustment of p-values for multiple testing was performed using the Benjamin-Hochberg false discovery rate method. For boxplots we used the ggpubr package to calculate p-values as indicated in the figures, and ggplot2 was used to generate plots. Kinase networks were analysed from phosphoproteomics data using an R implementation of KSEA (P. Casado et al., Sci Signal 6, rs6 (2013)) using markers of network edge provided by M. Hijazi et al. Nat Biotechnol, (2020). Values of kinase node centrality were derived using the page.rank function in the iGraph R package. Gene ontology and transcription factor (TF) target enrichment analysis was carried out using z-scores (as in the PAGE method) to determine extent of enrichment and the Kolmogorov-Smirnoff test (KS function in base R) for assessing the statistical significance of the enrichment. Ontology and TF-protein relationships were downloaded from Uniprot (https://www.uniprot.org/) and the Molecular Signatures Database (https://www.gsea-msigdb.org/gsea/msigdb/genesets.jsp), respectively. The R code that we used for KSEA, kinase network analysis, ontology and TF enrichment analysis is publicly available (M. Hijazi et al. Nat Biotechnol, (2020)), https://github.com/CutillasLab/ebdt).

The extent of drug interaction was calculated in Microsoft Excel 2016 using the CDI formula AB/(A*B), where AB is the normalized reduction in cell viability of the sequential treatment, and A and B are the effects of single treatments. Statistical significance of CDI values for each AML sample was determined using a one sample t-test (base R package) with the null hypothesis being that the mean Log₂(CDI) equals 0.

For machine learning, we used random forest regression modelling using the rf function in caret. Samples were split into training and validation sets (80:20 ratio) using the “createDataPartition” function. Resampling was done using 10-fold cross validation repeated 3 times using RMSE as the cost function. The final RF model consisted of 1000 trees.

1. A method of treating cancer in a subject in need thereof, comprising administering to the subject an epigenetic agent which inhibits a chromatin-modifying enzyme and/or which promotes cellular differentiation, and then subsequently administering a kinase inhibitor to the subject.

2. A method of sensitising a cancer to treatment with a kinase inhibitor wherein the cancer is pre-treated with an epigenetic agent which inhibits a chromatin-modifying enzyme and/or which promotes cellular differentiation.

3. A kinase inhibitor for use in a method of treating cancer in a subject in need thereof, wherein the subject has previously been administered an epigenetic agent which inhibits a chromatin-modifying enzyme and/or which promotes cellular differentiation.

4. An epigenetic agent for use in a method of sensitising a cancer to treatment with a kinase inhibitor wherein the method comprises pre-treating the cancer with an epigenetic agent which inhibits a chromatin-modifying enzyme and/or which promotes cellular differentiation.

5. A kit comprising an epigenetic agent which inhibits a chromatin-modifying enzyme and/or which promotes cellular differentiation, and a kinase inhibitor for simultaneous, separate or sequential use for the treatment of cancer.

6. The method, kinase inhibitor for use, epigenetic agent for use, or kit according to any preceding claim wherein the cancer comprises cells with one or more of the following markers:
(i) phosphorylation at one or more of the following phosphorylation sites:
Serine/threonine-protein kinase PAK 1 phosphorylated at serine 219;
Mitogen-activated protein kinase kinase kinase 2 phosphorylated at threonine 339;
Ribosomal protein S6 kinase alpha-3 phosphorylated at threonine 391;
Receptor tyrosine-protein kinase erbB-2 phosphorylated at serine 1073;
Mitogen-activated protein kinase kinase kinase 3 phosphorylated at serine 129;
Epidermal growth factor receptor phosphorylated at serine 991;
PH domain leucine-rich repeat-containing protein phosphatase 1 phosphorylated at serine 1524;
Dual specificity mitogen-activated protein kinase kinase 1 phosphorylated at serine 222;
Dual specificity mitogen-activated protein kinase kinase 1 phosphorylated at serine 226;
Kinase suppressor of Ras 1 phosphorylated at serine 202;
Dual specificity mitogen-activated protein kinase kinase 1 phosphorylated at serine 230;
Serine/threonine-protein kinase B-raf phosphorylated at threonine 753;
Mitogen-activated protein kinase kinase kinase 4 phosphorylated at serine 499;
Serine/threonine-protein kinase mTOR phosphorylated at threonine 2446;
Serine/threonine-protein kinase mTOR phosphorylated at serine 2448;
Serine/threonine-protein kinase A-Raf phosphorylated at serine 580;
RAF proto-oncogene serine/threonine-protein kinase phosphorylated at serine 619;
PH domain leucine-rich repeat-containing protein phosphatase 2 phosphorylated at serine 1210;
Mitogen-activated protein kinase kinase kinase 20 at serine 648;
Mitogen-activated protein kinase kinase kinase 20 at serine 649;
Dual specificity mitogen-activated protein kinase kinase 2 phosphorylated at threonine 13;
Glycogen synthase kinase-3 beta phosphorylated at serine 215;
Glycogen synthase kinase-3 alpha phosphorylated at serine 278;
Mitogen-activated protein kinase 3 phosphorylated at threonine 207;
Serine/threonine-protein kinase N1 phosphorylated at serine 584;
Tyrosine-protein phosphatase non-receptor type 7 phosphorylated at serine 359;
RAC-alpha serine/threonine-protein kinase phosphorylated at serine 457;
RAC-beta serine/threonine-protein kinase phosphorylated at serine 458;
RAC-beta serine/threonine-protein kinase phosphorylated at tyrosine 438;
Mitogen-activated protein kinase 3 phosphorylated at threonine 202;
Mitogen-activated protein kinase 3 phosphorylated at tyrosine 204;
Serine/threonine-protein kinase PAK 1 phosphorylated at threonine 229;
Serine/threonine-protein kinase PAK 1 phosphorylated at threonine 230;
Eukaryotic translation initiation factor 4E-binding protein 1 phosphorylated at threonine 77; and/or
Dual specificity mitogen-activated protein kinase kinase 1 phosphorylated at serine 377; and/or
(ii) expression of one or more of the following proteins:
Lysine-specific histone demethylase;
Serine/threonine-protein kinase mTOR;
Dual specificity mitogen-activated protein kinase kinase 2;
Ribosomal protein S6 kinase beta-1;
Mitogen-activated protein kinase 1;
Dual specificity mitogen-activated protein kinase kinase 4;
Ribosomal protein S6 kinase alpha-1;
Dual specificity mitogen-activated protein kinase kinase 3;
Ribosomal protein S6 kinase alpha-5;
Dual specificity mitogen-activated protein kinase kinase 5;
Phosphatidylinositol 4,5-bisphosphate 3-kinase catalytic subunit delta isoform; and/or
Dual specificity mitogen-activated protein kinase kinase 1; and/or
(iii) mutation of, or chromosomal rearrangement involving, one or more of the following genes: KMT2A, NPM1, KRAS, NRAS, DNMT3A, IDH2, TP53, TET2, FLT3 and/or STAG2; and/or
(iv) classified as M5 by the French-American-British (FAB) AML classification scheme.

7. A method of selecting a subject with cancer for treatment with an epigenetic agent which inhibits a chromatin-modifying enzyme and/or which promotes cellular differentiation, followed by treatment with a kinase inhibitor, wherein the method comprises:
detecting, in a sample of cancer cells obtained from the subject, the presence and/or level of one or more of the following markers:
(i) phosphorylation at one or more of the following phosphorylation sites:
Serine/threonine-protein kinase PAK 1 phosphorylated at serine 219;
Mitogen-activated protein kinase kinase kinase 2 phosphorylated at threonine 339;
Ribosomal protein S6 kinase alpha-3 phosphorylated at threonine 391;
Receptor tyrosine-protein kinase erbB-2 phosphorylated at serine 1073;
Mitogen-activated protein kinase kinase kinase 3 phosphorylated at serine 129;
Epidermal growth factor receptor phosphorylated at serine 991;
PH domain leucine-rich repeat-containing protein phosphatase 1 phosphorylated at serine 1524;
Dual specificity mitogen-activated protein kinase kinase 1 phosphorylated at serine 222;
Dual specificity mitogen-activated protein kinase kinase 1 phosphorylated at serine 226;
Kinase suppressor of Ras 1 phosphorylated at serine 202;
Dual specificity mitogen-activated protein kinase kinase 1 phosphorylated at serine 230;
Serine/threonine-protein kinase B-raf phosphorylated at threonine 753;
Mitogen-activated protein kinase kinase kinase 4 phosphorylated at serine 499;
Serine/threonine-protein kinase mTOR phosphorylated at threonine 2446;
Serine/threonine-protein kinase mTOR phosphorylated at serine 2448;
Serine/threonine-protein kinase A-Raf phosphorylated at serine 580;
RAF proto-oncogene serine/threonine-protein kinase phosphorylated at serine 619;
PH domain leucine-rich repeat-containing protein phosphatase 2 phosphorylated at serine 1210;
Mitogen-activated protein kinase kinase kinase 20 at serine 648;
Mitogen-activated protein kinase kinase kinase 20 at serine 649;
Dual specificity mitogen-activated protein kinase kinase 2 phosphorylated at threonine 13;
Glycogen synthase kinase-3 beta phosphorylated at serine 215;
Glycogen synthase kinase-3 alpha phosphorylated at serine 278;
Mitogen-activated protein kinase 3 phosphorylated at threonine 207;
Serine/threonine-protein kinase N1 phosphorylated at serine 584;
Tyrosine-protein phosphatase non-receptor type 7 phosphorylated at serine 106;
Tyrosine-protein phosphatase non-receptor type 7 phosphorylated at serine 359;
RAC-alpha serine/threonine-protein kinase phosphorylated at tyrosine 437 RAC-alpha serine/threonine-protein kinase phosphorylated at serine 457;
RAC-beta serine/threonine-protein kinase phosphorylated at serine 458;
RAC-beta serine/threonine-protein kinase phosphorylated at tyrosine 438;
Mitogen-activated protein kinase 3 phosphorylated at threonine 202;
Mitogen-activated protein kinase 3 phosphorylated at tyrosine 204;
Serine/threonine-protein kinase PAK 1 phosphorylated at threonine 229;
Serine/threonine-protein kinase PAK 1 phosphorylated at threonine 230;
Eukaryotic translation initiation factor 4E-binding protein 1 phosphorylated at threonine 77; and/or
Dual specificity mitogen-activated protein kinase kinase 1 phosphorylated at serine 377; and/or
(ii) expression of one or more of the following proteins:
Lysine-specific histone demethylase;
Serine/threonine-protein kinase mTOR;
Dual specificity mitogen-activated protein kinase kinase 2;
Ribosomal protein S6 kinase beta-1;
Mitogen-activated protein kinase 1;
Dual specificity mitogen-activated protein kinase kinase 4;
Ribosomal protein S6 kinase alpha-1;
Dual specificity mitogen-activated protein kinase kinase 3;
Ribosomal protein S6 kinase alpha-5;
Dual specificity mitogen-activated protein kinase kinase 5;
Phosphatidylinositol 4,5-bisphosphate 3-kinase catalytic subunit delta isoform; and/or
Dual specificity mitogen-activated protein kinase kinase 1; and/or
(iii) mutation of, or chromosomal rearrangement involving, one or more of the following genes: KMT2A, NPM1, KRAS, NRAS, DNMT3A, IDH2, TP53, TET2, FLT3 and/or STAG2; and/or
(iv) classified as M5 by the French-American-British (FAB) AML classification scheme
wherein detecting the presence and/or level of one or more of said markers selects the subject for treatment with the epigenetic agent followed by treatment with the kinase inhibitor.

8. The method of any one of claims 6 or 7 wherein:
Markers (i) comprise a high level of phosphorylation at one or more phosphorylation sites selected from the group consisting of:
Serine/threonine-protein kinase PAK 1 phosphorylated at serine 219;
Mitogen-activated protein kinase kinase kinase 2 phosphorylated at threonine 339;
Ribosomal protein S6 kinase alpha-3 phosphorylated at threonine 391;
Receptor tyrosine-protein kinase erbB-2 phosphorylated at serine 1073;
Mitogen-activated protein kinase kinase kinase 3 phosphorylated at serine 129;
Epidermal growth factor receptor phosphorylated at serine 991;
PH domain leucine-rich repeat-containing protein phosphatase 1 phosphorylated at serine 1524;
Dual specificity mitogen-activated protein kinase kinase 1 phosphorylated at serine 222;
Dual specificity mitogen-activated protein kinase kinase 1 phosphorylated at serine 226;
Kinase suppressor of Ras 1 phosphorylated at serine 202;
Dual specificity mitogen-activated protein kinase kinase 1 phosphorylated at serine 230;
Dual specificity mitogen-activated protein kinase kinase 2 phosphorylated at threonine 13;
Mitogen-activated protein kinase 3 phosphorylated at threonine 207;
Serine/threonine-protein kinase N1 phosphorylated at serine 584;
Tyrosine-protein phosphatase non-receptor type 7 phosphorylated at serine 106;
Tyrosine-protein phosphatase non-receptor type 7 phosphorylated at serine 359;
RAC-alpha serine/threonine-protein kinase phosphorylated at tyrosine 437;
RAC-alpha serine/threonine-protein kinase phosphorylated at serine 457;
RAC-beta serine/threonine-protein kinase phosphorylated at serine 458;
RAC-beta serine/threonine-protein kinase phosphorylated at tyrosine 438;
Mitogen-activated protein kinase 3 phosphorylated at threonine 202;
Mitogen-activated protein kinase 3 phosphorylated at tyrosine 204;
Serine/threonine-protein kinase PAK 1 phosphorylated at threonine 229;
Serine/threonine-protein kinase PAK 1 phosphorylated at threonine 230;
Eukaryotic translation initiation factor 4E-binding protein 1 phosphorylated at threonine 77;
and/or
Dual specificity mitogen-activated protein kinase kinase 1 phosphorylated at serine 377;
and/or
Markers (i) comprise a low level or not a high level of phosphorylation at one or more phosphorylation sites selected from the group consisting of:
Glycogen synthase kinase-3 beta phosphorylated at serine 215;
Glycogen synthase kinase-3 alpha phosphorylated at serine 278;
Serine/threonine-protein kinase B-raf phosphorylated at threonine 753;
Mitogen-activated protein kinase kinase kinase 4 phosphorylated at serine 499;
Serine/threonine-protein kinase mTOR phosphorylated at threonine 2446;
Serine/threonine-protein kinase mTOR phosphorylated at serine 2448;
Serine/threonine-protein kinase A-Raf phosphorylated at serine 580;
RAF proto-oncogene serine/threonine-protein kinase phosphorylated at serine 619;
PH domain leucine-rich repeat-containing protein phosphatase 2 phosphorylated at serine 1210;
Mitogen-activated protein kinase kinase kinase 20 at serine 648; and/or
Mitogen-activated protein kinase kinase kinase 20 at serine 649;
and/or
Markers (ii) comprise a high level of one or more proteins selected from the group consisting of:
Lysine-specific histone demethylase;
Dual specificity mitogen-activated protein kinase kinase 2;
Ribosomal protein S6 kinase beta-1;
Mitogen-activated protein kinase 1;
Dual specificity mitogen-activated protein kinase kinase 4;
Ribosomal protein S6 kinase alpha-1;
Dual specificity mitogen-activated protein kinase kinase 3;
Phosphatidylinositol 4,5-bisphosphate 3-kinase catalytic subunit delta isoform; and/or
Dual specificity mitogen-activated protein kinase kinase 1;
and/or
Markers (ii) comprise a low level or not a high level of one or more proteins selected from the group consisting of:
Ribosomal protein S6 kinase alpha-5;
Serine/threonine-protein kinase mTOR; and/or
Dual specificity mitogen-activated protein kinase kinase 5,
and/or
Markers (iii) comprise a mutation of, or chromosomal rearrangement involving, one or more of the following genes:
KMT2A and/or KRAS;
and/or
Markers (iii) comprise the lack of a mutation of, or chromosomal rearrangement involving, one or more of the following genes:
NPM1, NRAS and/or DNMT3A.

9. The method of any one of claims 6 to 8 wherein:
Markers (i) comprise a high level of phosphorylation at one or more phosphorylation sites selected from the group consisting of:
Serine/threonine-protein kinase PAK 1 phosphorylated at serine 219;
Mitogen-activated protein kinase 3 phosphorylated at threonine 207;
Serine/threonine-protein kinase N1 phosphorylated at serine 584;
Tyrosine-protein phosphatase non-receptor type 7 phosphorylated at serine 106;
Tyrosine-protein phosphatase non-receptor type 7 phosphorylated at serine 359;
RAC-alpha serine/threonine-protein kinase phosphorylated at tyrosine 437;
RAC-alpha serine/threonine-protein kinase phosphorylated at serine 457;
RAC-beta serine/threonine-protein kinase phosphorylated at serine 458;
RAC-beta serine/threonine-protein kinase phosphorylated at tyrosine 438;
Mitogen-activated protein kinase 3 phosphorylated at threonine 202;
Mitogen-activated protein kinase 3 phosphorylated at tyrosine 204;
Serine/threonine-protein kinase PAK 1 phosphorylated at threonine 229;
Serine/threonine-protein kinase PAK 1 phosphorylated at threonine 230; and/or
Eukaryotic translation initiation factor 4E-binding protein 1 phosphorylated at threonine 77;
and/or
Markers (i) comprise a low level or not a high level of phosphorylation at one or more phosphorylation sites selected from the group consisting of:
Glycogen synthase kinase-3 beta phosphorylated at serine 215; and/or
Glycogen synthase kinase-3 alpha phosphorylated at serine 278;
and/or
Markers (ii) comprise a high level of one or more proteins selected from the group consisting of:
Lysine-specific histone demethylase;
Dual specificity mitogen-activated protein kinase kinase 2;
Dual specificity mitogen-activated protein kinase kinase 4;
Ribosomal protein S6 kinase alpha-1;
Dual specificity mitogen-activated protein kinase kinase 3;
Phosphatidylinositol 4,5-bisphosphate 3-kinase catalytic subunit delta isoform; and/or
Dual specificity mitogen-activated protein kinase kinase 1;
and/or
Markers (ii) comprise a low level or not a high level of Ribosomal protein S6 kinase alpha-5.

10. The method of any one of claims 6 to 9 wherein:
(a) the mutation or chromosomal rearrangement of KMT2A is one or more mutation or chromosomal rearrangement selected from the group consisting of a KMT2A-MLLT10 Rearrangement, a KMT2A-MLLT6 Rearrangement, a KMT2A-AFDN Rearrangement and a KMT2A-R2477Q Point mutation;
(b) the mutation or chromosomal rearrangement of KRAS is one or more point mutation selected from the group consisting of a KRAS-G12P Point mutation, a KRAS-G12A Point mutation and a KRAS-Q61R Point mutation;
(c) the mutation or chromosomal rearrangement of NRAS is one or more point mutation selected from the group consisting of a NRAS-G12D Point mutation, a NRAS-G13D Point mutation and a NRAS-Q61P Point mutation;
(d) the mutation or chromosomal rearrangement of NPM1 is one or more insertion selected from the group consisting of a NPM1-L287 Insertion (TCTG), a NPM1-L287 Insertion (TGCA) and a NPM1-W288 Insertion (GCTT);
(e) the mutation or chromosomal rearrangement of DNMT3A is one or more point mutation or splice donor variant selected from the group consisting of a DNMT3A-R882H Point mutation and a DNMT3A Splice donor variant;
(f) the mutation or chromosomal rearrangement of IDH2 is a IDH2-R140Q Point mutation;
(g) the mutation or chromosomal rearrangement of TP53 is one or more point mutation selected from the group consisting of a TP53-C106Y Point mutation and a TP53-A159P Point mutation;
(h) the mutation or chromosomal rearrangement of TET2 is one or more point mutation or insertion selected from the group consisting of a TET2-C1263Y Point mutation, a TET2-D1113 Insertion (AT) and a TET2-H924R Point mutation;
(i) the mutation or chromosomal rearrangement of FLT3 is one or more point mutation or insertion selected from the group consisting of a FLT3-D835Y Point mutation, a FLT3-D835H Point mutation and a FLT3-internal tandem duplication (ITD) Insertion; and/or
(j) the mutation or chromosomal rearrangement of STAG2 is a STAG2-M930 Deletion.

11. The method of any one of claims 6 to 10 wherein comprising performing an in vitro assay to detect the presence and/or level of one or more markers in the sample obtained from the patient.

12. The method of claim 11 wherein the assay is an LC-MS/MS assay or an assay based on affinity reagents such as aptamers, molecularly imprinted polymers, or antibodies, optionally wherein the assay based on affinity reagents is a Western blot assay, an ELISA assay or a reversed phase protein assay.

13. The method, kinase inhibitor for use, epigenetic agent for use, or kit according to any preceding claim wherein the chromatin-modifying enzyme is lysine-specific histone demethylase 1A (LSD1, also known as KDM1A), DNA methyltransferase (DNMTA), histone deacetylase (HDAC), enhancer of zeste homolog 2 (EZH2) or disruptor of telomeric silencing 1-like protein (DOT1L), preferably wherein the chromatin-modifying enzyme is LSD1.

14. The method, kinase inhibitor for use, epigenetic agent for use, or kit according to any preceding claim wherein the epigenetic agent is selected from GSK2879552, ORY1001, GSK-LSD1, CC90011, IMG-7289 (Bomedemstat), all-trans retinoic acid (ATRA), decitabine, vorinostat, CPI-1205 and pinometostat, preferably wherein the epigenetic agent is GSK2879552, ORY1001, GSK-LSD1 or CC90011.

15. The method, kinase inhibitor for use, epigenetic agent for use, or kit according to any preceding claim wherein the kinase inhibitor inhibits a kinase selected from the group consisting of mitogen-activated protein kinase kinase 1/2 (MEK1 or MEK2), mammalian target of rapamycin kinase (mTOR), protein kinase C (PKC), FMS-like tyrosine kinase 3 (FLT-3), phosphoinositide 3-kinase (PI3K), casein kinase 2 (CK2), AKT1, AKT2 and mammalian target of rapamycin (mTOR).

16. The method, kinase inhibitor for use, epigenetic agent for use, or kit according to claim 15 wherein the kinase is selected from the group consisting of MEK1 or MEK2, their downstream and upstream effectors ARAF, BRAF, CRAF, ribosomal S6 kinases (RPS6KA1, RPS6KA2, RPS6KA3), and extracellular signal-regulated kinases 1 and 2 (ERK1 and ERK2, gene names MAPK3 and MAPK1, respectively).

17. The method, kinase inhibitor for use, epigenetic agent for use, or kit according to any preceding claim wherein the kinase inhibitor is selected from trametinib, midostaurin, pictilisib, torin 1 and silmitasertib, preferably wherein the kinase inhibitor is trametinib.

18. The method, kinase inhibitor for use, epigenetic agent for use, or kit according to any preceding claim wherein the cancer is selected from acute myeloid leukaemia, oesophageal cancer, breast cancer and liver cancer.

19. The method, kinase inhibitor for use, epigenetic agent for use, or kit according to any preceding claim wherein the cancer is acute myeloid leukaemia.

20. The method, kinase inhibitor for use, epigenetic agent for use, or kit according to any preceding claim wherein the cancer is classified as being a FAB M4, M5 or M6 subtype acute myeloid leukaemia.

21. The method, kinase inhibitor for use, epigenetic agent for use, or kit according to any preceding claim wherein the cancer is classified as being KMT2A rearranged (KMT2Ar) acute lymphoblastic leukaemia (ALL).

22. The method, kinase inhibitor for use, epigenetic agent for use, or kit according to any preceding claim wherein the cancer, prior to treatment with the epigenetic agent, displayed resistance to treatment with the kinase inhibitor.

23. The method, kinase inhibitor for use, epigenetic agent for use, or kit according to any preceding claim wherein:
(i) the cancer is acute myeloid leukaemia, optionally wherein the cancer is classified as being a FAB M4, M5 or M6 subtype acute myeloid leukaemia or as KMT2A rearranged (KMT2Ar) acute lymphoblastic leukaemia (ALL);
(ii) the epigenetic agent is ATRA or inhibits LSD1, optionally wherein the epigenetic agent is GSK2879552; and
(iii) the kinase inhibitor inhibits MEK, optionally wherein the kinase inhibitor is trametinib.

24. The method, kinase inhibitor for use, epigenetic agent for use, or kit according to any preceding claim wherein the cancer comprises:
(a) cells expressing one or more of the following cluster of differentiation proteins: CD11b, CD64, CD14, CD117, CD16 and/or CD15; and/or
(b) higher activity of AKT1, PI3KCA and/or MAPK1/3 signalling pathways and/or higher phosphorylation of one or more MAPK and PI3K/mTOR pathway activation markers, such as PAK1 at T219, MEK2 at S23, MAPK3 at T202/T207/Y204 and AKT1S1 (PRAS40) at S183, relative to a threshold level per marker; and/or
(c) Ras mutations or increased protein expression of one or more targets of Ras downstream targets, such as MP2K5, (MEK1 (MAPK2K1), MEK2 (MAP2K2), MAPK1, MAPK3, and ribosomal S6 kinase relative to a threshold level per marker.

25. The method, kinase inhibitor for use, epigenetic agent for use, or kit according to claim 24(a) wherein:
(i) if the cancer comprises cells expressing CD11b and/or CD64 then the kinase inhibitor is trametinib; or
(ii) if the cancer comprises cells expressing CD117, CD16 and/or CD15 then the kinase inhibitor is silmitasertib.


SEQUENCE LISTING (37 sequences)

SEQ ID NO: 1 - protein, 20 aa, Homo sapiens
RGSDIDNPTLTVMDISPPSR

SEQ ID NO: 2 - protein, 30 aa, Homo sapiens
GFSFVAITSDDESQAMQTVGVHSIVQQLHR

SEQ ID NO: 3 - protein, 24 aa, Homo sapiens
SPLAPSEGAGSDVFDGDLGMGAAK

SEQ ID NO: 4 - protein, 13 aa, Homo sapiens
NHNSSSPHSGVSR

SEQ ID NO: 5 - protein, 13 aa, Homo sapiens
MHLPSPTDSNFYR

SEQ ID NO: 6 - protein, 14 aa, Homo sapiens
CMLHPICLSNSFQR

SEQ ID NO: 7 - protein, 22 aa, Homo sapiens
LCDFGVSGQLIDSMANSFVGTR

SEQ ID NO: 8 - protein, 22 aa, Homo sapiens
LCDFGVSGQLIDSMANSFVGTR

SEQ ID NO: 9 - protein, 28 aa, Homo sapiens
RESGSGPSTDTLSAASLPWPPGSSQLGR

SEQ ID NO: 10 - protein, 22 aa, Homo sapiens
LCDFGVSGQLIDSMANSFVGTR

SEQ ID NO: 11 - protein, 14 aa, Homo sapiens
TPIQAGGYGAFPVH

SEQ ID NO: 12 - protein, 23 aa, Homo sapiens
LESEDDSLGWGAPDWSTEAGFSR

SEQ ID NO: 13 - protein, 25 aa, Homo sapiens
TDSYSAGQSVEILDGVELGEPAHKK

SEQ ID NO: 14 - protein, 25 aa, Homo sapiens
TDSYSAGQSVEILDGVELGEPAHKK

SEQ ID NO: 15 - protein, 9 aa, Homo sapiens
SASEPSLHR

SEQ ID NO: 16 - protein, 9 aa, Homo sapiens
SASEPSLHR

SEQ ID NO: 17 - protein, 14 aa, Homo sapiens
QNSVNSGMLLPMSK

SEQ ID NO: 18 - protein, 10 aa, Homo sapiens
NFSSLHLNSR

SEQ ID NO: 19 - protein, 10 aa, Homo sapiens
NFSSLHLNSR

SEQ ID NO: 20 - protein, 35 aa, Homo sapiens
RKPVLPALTINPTIAEGPSPTSEGASEANLVDLQK

SEQ ID NO: 21 - protein, 15 aa, Homo sapiens
QLVRGEPNVSYICSR

SEQ ID NO: 22 - protein, 15 aa, Homo sapiens
QLVRGEPNVSYICSR

SEQ ID NO: 23 - protein, 19 aa, Homo sapiens
IADPEHDHTGFLTEYVATR

SEQ ID NO: 24 - protein, 41 aa, Homo sapiens
SSRDPPSSPSSLSSPIQESTAPELPSETQETPGPALCSPLR

SEQ ID NO: 25 - protein, 19 aa, Homo sapiens
IPSNFVSPEDLDIPGHASK

SEQ ID NO: 26 - protein, 30 aa, Homo sapiens
GGMIQTAEQYQFLHHTLALYAGQLPEEPSP

SEQ ID NO: 27 - protein, 29 aa, Homo sapiens
YFDEEFTAQMITITPPDQDDSMECVDSER

SEQ ID NO: 28 - protein, 29 aa, Homo sapiens
YFDEEFTAQMITITPPDQDDSMECVDSER

SEQ ID NO: 29 - protein, 30 aa, Homo sapiens
YFDDEFTAQSITITPPDRYDSLGLLELDQR

SEQ ID NO: 30 - protein, 30 aa, Homo sapiens; identical to SEQ ID NO: 29

SEQ ID NO: 31 - protein, 19 aa, Homo sapiens
IADPEHDHTGFLTEYVATR

SEQ ID NO: 32 - protein, 19 aa, Homo sapiens
IADPEHDHTGFLTEYVATR

SEQ ID NO: 33 - protein, 22 aa, Homo sapiens
DVATSPISPTENNTTPPDALTR

SEQ ID NO: 34 - protein, 22 aa, Homo sapiens
DVATSPISPTENNTTPPDALTR

SEQ ID NO: 35 - protein, 22 aa, Homo sapiens
DVATSPISPTENNTTPPDALTR

SEQ ID NO: 36 - protein, 26 aa, Homo sapiens
DLPTIPGVTSPSSDEPPMEASQSHLR

SEQ ID NO: 37 - protein, 31 aa, Homo sapiens
RSDAEEVDFAGWLCSTIGLNQPSTPTHAAGV